(19) United States
(12) Patent Application Publication
(10) Pub. No.: US 2026/0360468 A1
(43) Pub. Date: Nov. 12, 2026

(54) METHODS AND SYSTEMS FOR DETERMINING FEATURES OF FILTERED CFDNA FRAGMENTS

(71) Applicant: Foundation Medicine, Inc., Boston, MA (US)

(72) Inventors: Kung-Yen CHANG, San Diego, CA (US); Ray Abrams GREENSTEIN, West Hartford, CT (US); Michael PETERS, Cambridge, MA (US); Haleh ASHKI, Escondido, CA (US); Mark R. KENNEDY, Cambridge, MA (US)

(73) Assignee: Foundation Medicine, Inc., Boston, MA (US)

(21) Appl. No.: 19/671,035

(22) Filed: May 7, 2026

Related U.S. Application Data
(60) Provisional application No. 63/802,362, filed on May 8, 2025.

(51) Int. Cl. G16B 30/10; G16B 40/20; G16B 40/30; G16H 50/20

(52) U.S. Cl. CPC G16B 30/10; G16B 40/20; G16B 40/30; G16H 50/20

(57) ABSTRACT

Methods for training a filter model for filtering sequencing data for cell-free DNA (cfDNA) fragments, determining features of cfDNA fragments, and filtering and labeling cfDNA fragments as associated with disease or not are described. The methods may comprise, for example, training a filter model comprising receiving sequencing data for a plurality of sample cfDNA fragments; generating a value indicating that each of one or more sample cfDNA fragments originated from tissue not impacted by disease; determining a methylation fraction difference between control cfDNA fragments and each of the one or more sample cfDNA fragments; determining a fragment length difference between control cfDNA fragments and each of the one or more sample cfDNA fragments; determining a subgenomic interval-specific threshold for each of a plurality of subgenomic intervals for the values, methylation fraction differences, and fragment length differences; and setting thresholds of a filter model using the subgenomic interval-specific thresholds.

### CROSS-REFERENCE TO RELATED APPLICATIONS

This application claims priority to U.S. Provisional Patent Application No. 63/802,362, filed on May 8, 2025, the disclosure of which is hereby incorporated by reference in its entirety.

### FIELD

The present disclosure relates to methods and systems for filtering and analyzing sequencing data for cell-free DNA (cfDNA) fragments to identify an association with disease, and more specifically to methods and systems for training a model for filtering sequencing data for cell-free DNA (cfDNA) fragments, labeling cfDNA fragments as associated with disease or not, and determining a most likely type of disease using trained filter models. Also described herein are methods and systems that use trained filter models and labeled cfDNA fragments for diagnosing or treating disease or selecting therapies to treat disease.

### BACKGROUND

DNA methylation in cell-free DNA (cfDNA) is a valuable tool for detecting disease in subjects using a minimally invasive sample. Extracting cfDNA fragments can be relatively non-invasive compared to other methods of DNA extraction, and in some diseases, there is no specific tissue that can be used to assess disease. Analysis of cfDNA fragments and features of cfDNA fragments may be used for disease detection, therapy selection, therapy response monitoring, and/or recurrence monitoring. Samples may contain cfDNA fragments associated with disease because they are derived from diseased cells or tissue, or because they are derived from healthy cells or tissue impacted by disease. For example, cfDNA fragments in samples may be from cells related to the body's response to disease, such as immune cells or cells from the tumor microenvironment. However, samples often also contain an overwhelming majority of cfDNA fragments derived from healthy cells or tissue and not associated with disease. Issues relating to identifying the origin of cfDNA and a possible disease association can lead to challenges in assay sensitivity for disease detection, especially in early stages or early parts of relapse of cancer, and diminished accuracy in tissue or tumor of origin detection. Existing methods have attempted to extract information from cfDNA fragments by analyzing a singular feature to identify cfDNA as associated with disease. For example, some existing methods aim to predict an association with disease by analyzing only the methylation status of cfDNA fragments. However, these approaches may be prone to error, limited, and unreliable, especially in cases where the signal is weak. Thus, there is an ongoing need to develop methods and systems that can distinguish between cfDNA that originated from cells or tissue not impacted by disease, and cfDNA that originated from cells or tissue impacted by disease.

### BRIEF SUMMARY OF THE INVENTION

Disclosed herein are methods and systems for filtering sequencing data for cfDNA fragments, labeling cfDNA fragments as originating from tissue impacted by disease or not originating from tissue impacted by disease, determining features of filtered cfDNA fragments. While it is valuable to analyze cfDNA fragments in minimally invasive samples, there is often an overwhelming majority of cfDNA fragments that are not associated with disease. Because of this, it is challenging to identify cfDNA fragments that likely originate from diseased tissue or healthy tissue impacted by disease, rather than cfDNA fragments that likely originate from healthy cells not impacted by disease. Existing attempts to resolve this issue work only at high tumor fractions and are limited by the variability of noise in samples.

The present invention can be used to improve detection sensitivity for disease or its relapse and to improve accuracy in tissue of origin determination. In some aspects, the present invention may be used to identify cfDNA fragments associated with disease that are distinct from cfDNA fragments from a healthy cell background not impacted by disease. The disclosed methods comprise measuring features or features of cfDNA fragments in a wide range of genomic contexts or subgenomic intervals, and then using those measurements to threshold and filter cfDNA fragments that are associated with disease from cfDNA fragments that are not associated with disease. The disclosed methods also comprise using filter thresholds to separate unexpected cfDNA fragments that are likely to be informative about disease, from a large background of cfDNA fragments that are not associated with disease. Once thresholds have been set in local genomic contexts, they may be applied to classify test cfDNA fragments as associated with disease or not. From this filtering process, the disclosed methods include computing various metrics within biological contexts or subgenomic intervals that apply to the filtered cfDNA fragments predicted to be associated with disease. Information about the filtered cfDNA fragments may also be used to predict, in addition to the presence of disease, a specific type of disease. Filtered cfDNA fragment metrics may be used as features for use in downstream classifier models for further applications in disease detection.

In some aspects, disclosed herein is a method of training a filter model for filtering cfDNA fragments, comprising: receiving, at one or more processors, sample sequencing data for a plurality of sample cfDNA fragments; determining, using the one or more processors, for each of one or more sample cfDNA fragments from the plurality of sample cfDNA fragments, at least one of: a value indicating that the sample cfDNA fragment originated from tissue not impacted by a disease; a methylation fraction difference between a methylation fraction for corresponding control cfDNA fragments and a methylation fraction for the sample cfDNA fragment, and a fragment length difference between an average length of corresponding control cfDNA fragments and a fragment length of the sample cfDNA fragment; determining, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and setting, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

In some aspects, the plurality of sample cfDNA fragments are obtained from a training sample of a training subject having a disease. In some aspects, the method further comprises: determining, using the one or more processors, for each of one or more sample cfDNA fragments from the plurality of sample cfDNA fragments, at least two of: a value indicating that the sample cfDNA fragment originated from tissue not impacted by a disease; a methylation fraction difference between a methylation fraction for corresponding control cfDNA fragments and a methylation fraction for the sample cfDNA fragment, and a fragment length difference between an average length of corresponding control cfDNA fragments and a fragment length of the sample cfDNA fragment. In some aspects, the method further comprises: determining, using the one or more processors, for each of a plurality of subgenomic intervals, at least two of a subgenomic interval-specific value threshold, a subgenomic interval-specific methylation fraction difference threshold, and a subgenomic interval-specific fragment length difference threshold. In some aspects, the method further comprises: setting, using the one or more processors, one or more thresholds of a filter model based on at least two of the determined subgenomic interval-specific value threshold, the determined subgenomic interval-specific methylation fraction difference threshold, and the determined subgenomic interval-specific fragment length threshold.

In some aspects, the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length. In some aspects, the value is a statistical value indicating the probability that the sample cfDNA fragment originated from tissue not impacted by a disease, and the statistical value is based on the methylation status for the one or more first selected methylation sites in the sample cfDNA fragment and a methylation status for one or more third selected methylation sites in corresponding control cfDNA fragments. In some aspects, the methylation fraction difference is between the methylation fraction for one or more fourth selected methylation sites in the corresponding control cfDNA fragments and the methylation fraction for one or more second selected methylation sites in the sample cfDNA fragment.

In some aspects, disclosed herein is a method of training a filter model for filtering cfDNA fragments, comprising: (a) receiving, at one or more processors, sample sequencing data for a plurality of sample cfDNA fragments obtained from a training sample of a training subject having a disease, the sample sequencing data comprising, for each of one or more sample cfDNA fragments from the plurality of sample cfDNA fragments, a methylation status for one or more first selected methylation sites, a methylation fraction for one or more second selected methylation sites, and a fragment length; (b) generating, using the one or more processors, for each of the one or more sample cfDNA fragments, a value indicating that the sample cfDNA fragment originated from tissue not impacted by the disease based on the methylation status for the one or more first selected methylation sites in the sample cfDNA fragment and a methylation status for one or more third selected methylation sites in corresponding control cfDNA fragments; (c) determining, using the one or more processors, for each of the one or more sample cfDNA fragments, a methylation fraction difference between a methylation fraction for one or more fourth selected methylation sites in corresponding control cfDNA fragments and the methylation fraction for the one or more second selected methylation sites in the sample cfDNA fragment; (d) determining, using the one or more processors, for each of the one or more sample cfDNA fragments, a fragment length difference between an average length of corresponding control cfDNA fragments and the fragment length of the sample cfDNA fragment; (e) determining, using the one or more processors, for each of a plurality of subgenomic intervals, a subgenomic interval-specific value threshold, a subgenomic interval-specific methylation fraction difference threshold, and a subgenomic interval-specific fragment length difference threshold based on the values, methylation fraction differences, and fragment length differences of each of the one or more sample cfDNA fragments that align with the subgenomic interval; and (f) setting, using the one or more processors, one or more thresholds of a filter model using the determined subgenomic interval-specific value thresholds, the determined subgenomic interval-specific methylation fraction difference thresholds, and the determined subgenomic interval-specific fragment length thresholds.

In some aspects, the value is a statistical value indicating the probability that the sample cfDNA fragment originated from tissue not impacted by the disease. In some aspects, the one or more first selected methylation sites are all the methylation sites in the sample cfDNA fragment, and the one or more third selected methylation sites are all the methylation sites in the corresponding control cfDNA fragments. In some aspects, the one or more first selected methylation sites are all the methylation sites in a sample cfDNA fragment, and the one or more third selected methylation sites are selected based on a positional overlap with the one or more first selected methylation sites in the sample cfDNA fragment and alignment within a common subgenomic interval with the sample cfDNA fragment. In some aspects, the one or more third selected methylation sites are all the methylation sites in the corresponding control cfDNA fragments, and the one or more first selected methylation sites are selected based on a positional overlap with the one or more third selected methylation sites in the corresponding control cfDNA fragments and alignment within a common subgenomic interval with the corresponding control cfDNA fragments. In some aspects, the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment. In some aspects, the one or more second selected methylation sites are all the methylation sites in the sample cfDNA fragment, and the one or more fourth selected methylation sites are all the methylation sites in the corresponding control cfDNA fragments. In some aspects, the one or more second selected methylation sites are all the methylation sites in a sample cfDNA fragment, and the one or more fourth selected methylation sites are selected based on a positional overlap with the one or more second selected methylation sites in the sample cfDNA fragment and alignment within a common subgenomic interval with the sample cfDNA fragment. In some aspects, the one or more fourth selected methylation sites are all the methylation sites in the corresponding control cfDNA fragments, and the one or more second selected methylation sites are selected based on a positional overlap with the one or more fourth selected methylation sites in the corresponding control cfDNA fragments and alignment within a common subgenomic interval with the corresponding control cfDNA fragments. In some aspects, the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment.

In some aspects, disclosed herein is a method of labeling test cfDNA fragments from a test subject, comprising: receiving, at one or more processors, test sequencing data for a plurality of test cfDNA fragments; determining, using the one or more processors, for each of one or more test cfDNA fragments from the plurality of test cfDNA fragments, at least one of: a value indicating that the test cfDNA fragment originated from tissue not impacted by the disease; a methylation fraction difference between a methylation fraction for corresponding control cfDNA fragments and a methylation fraction for the test cfDNA fragment; a fragment length difference between an average length of corresponding control cfDNA fragments and a fragment length of the test cfDNA fragment; inputting, using the one or more processors, for each of the one or more test cfDNA fragments, at least one of the value, the methylation fraction difference, and the fragment length difference into a trained filter model; filtering, using the one or more processors, the plurality of test cfDNA fragments using the filter model; and labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

In some aspects, the plurality of test cfDNA fragments are obtained from a test sample of a test subject. In some aspects, the selected corresponding control cfDNA fragments are selected based on alignment within a common subgenomic interval with the test cfDNA fragment. In some aspects, the method further comprises: determining, using the one or more processors, for each of one or more test cfDNA fragments from the plurality of test cfDNA fragments, at least two of: a value indicating that the test cfDNA fragment originated from tissue not impacted by a disease; a methylation fraction difference between a methylation fraction for corresponding control cfDNA fragments and a methylation fraction for the test cfDNA fragment, and a fragment length difference between an average length of corresponding control cfDNA fragments and a fragment length of the test cfDNA fragment. In some aspects, the method further comprises: determining, using the one or more processors, for each of a plurality of subgenomic intervals, at least two of a subgenomic interval-specific value threshold, a subgenomic interval-specific methylation fraction difference threshold, and a subgenomic interval-specific fragment length difference threshold. In some aspects, the method further comprises: setting, using the one or more processors, one or more thresholds of a filter model based on at least two of the determined subgenomic interval-specific value threshold, the determined subgenomic interval-specific methylation fraction difference threshold, and the determined subgenomic interval-specific fragment length threshold.

In some aspects, the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length. In some aspects, the value is a statistical value indicating the probability that the sample cfDNA fragment originated from tissue not impacted by a disease, and the statistical value is based on the methylation status for the one or more first selected methylation sites in the test cfDNA fragment and a methylation status for one or more third selected methylation sites in corresponding control cfDNA fragments. In some aspects, the methylation fraction difference is between the methylation fraction for one or more fourth selected methylation sites in the corresponding control cfDNA fragments and the methylation fraction for one or more second selected methylation sites in the test cfDNA fragment.

In some aspects, disclosed herein is a method of labeling test cfDNA fragments from a test subject, comprising: receiving, at one or more processors, test sequencing data comprising, for each of one or more test cfDNA fragments from a plurality of test cfDNA fragments from a test sample from the test subject, a methylation status for one or more first selected methylation sites, a methylation fraction for one or more second selected methylation sites, and a fragment length; generating, using the one or more processors, for each of the one or more test cfDNA fragments, a value indicating that the test cfDNA fragment originated from tissue not impacted by the disease based on the methylation status for the one or more first selected methylation sites in the test cfDNA fragment and a methylation status for one or more third selected methylation sites in corresponding control cfDNA fragments; determining, using the one or more processors, for each of the one or more test cfDNA fragments, a methylation fraction difference between a methylation fraction for one or more fourth selected methylation sites in the corresponding control cfDNA fragments and the methylation fraction for the one or more second selected methylation sites in the test cfDNA fragment; determining, using the one or more processors, for each of the one or more test cfDNA fragments, a fragment length difference between an average length of corresponding control cfDNA fragments and the fragment length of the test cfDNA fragment; inputting, using the one or more processors, for each of the one or more test cfDNA fragments, the value, the methylation fraction difference, and the fragment length difference into a trained filter model; filtering, using the one or more processors, the plurality of test cfDNA fragments using the filter model; and labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

In some aspects, the value is a statistical value indicating the probability that the test cfDNA fragment originated from tissue not impacted by the disease. In some aspects, the one or more first selected methylation sites are all the methylation sites in the test cfDNA fragment, and the one or more third selected methylation sites are all the methylation sites in the corresponding control cfDNA fragments. In some aspects, the one or more first selected methylation sites are all the methylation sites in a test cfDNA fragment, and the one or more third selected methylation sites are selected based on a positional overlap with the one or more first selected methylation sites in the test cfDNA fragment and alignment within a common subgenomic interval with the test cfDNA fragment. In some aspects, the one or more third selected methylation sites are all the methylation sites in the corresponding control cfDNA fragments, and the one or more first selected methylation sites are selected based on a positional overlap with the one or more third selected methylation sites in the corresponding control cfDNA fragments and alignment within a common subgenomic interval with the corresponding control cfDNA fragments. In some aspects, the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment. In some aspects, the one or more second selected methylation sites are all the methylation sites in the test cfDNA fragment, and the one or more fourth selected methylation sites are all the methylation sites in the corresponding control cfDNA fragments. In some aspects, the one or more second selected methylation sites are all the methylation sites in a test cfDNA fragment, and the one or more fourth selected methylation sites are selected based on a positional overlap with the one or more second selected methylation sites in the test cfDNA fragment and alignment within a common subgenomic interval with the test cfDNA fragment. In some aspects, the one or more fourth selected methylation sites are all the methylation sites in the corresponding control cfDNA fragments, and the one or more second selected methylation sites are selected based on a positional overlap with the one or more fourth selected methylation sites in the corresponding control cfDNA fragments and alignment within a common subgenomic interval with the corresponding control cfDNA fragments. In some aspects, the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment.

In some aspects, the method comprises sequencing the test cfDNA fragments to obtain the test sequencing data. In some aspects, the test sequencing data was obtained by: providing a plurality of test cfDNA fragments obtained from a test sample from a test subject; ligating one or more adapters onto one or more test cfDNA fragments from the plurality of test cfDNA fragments; amplifying the one or more ligated test cfDNA fragments from the plurality of test cfDNA fragments; capturing amplified test cfDNA fragments from the amplified test cfDNA fragments; sequencing, by a sequencer, the captured test cfDNA fragments to obtain a plurality of sequence reads that represent the captured test cfDNA fragments. In some aspects, the captured test cfDNA fragments are captured from the amplified test cfDNA fragments by hybridization to one or more bait molecules. In some aspects, the one or more bait molecules comprise one or more cfDNA fragments, each comprising a region that is complementary to a region of a captured test cfDNA fragment.

In some aspects, generating the statistical value comprises: aligning, using the one or more processors, the test cfDNA fragment and the plurality of corresponding control cfDNA fragments to a reference sequence to generate an alignment; identifying from the alignment, using the one or more processors, corresponding control cfDNA fragments that positionally overlap with the test cfDNA fragment; and comparing, using the one or more processors, the methylation status for the one or more first selected methylation sites in the test cfDNA fragment to the methylation status for the one or more third selected methylation sites in the corresponding control cfDNA fragments. In some aspects, comparing the methylation status for the one or more first selected methylation sites in the test cfDNA fragment to the methylation status for one or more third selected methylation sites in the corresponding control cfDNA fragments comprises determining a number of methylated sites in the one or more first selected methylation sites in the test cfDNA fragment, a number of unmethylated sites in the one or more first selected methylation sites in the test cfDNA fragment, a number of methylated sites in the one or more third selected methylation sites in the corresponding control cfDNA fragments, and a number of unmethylated sites in the one or more third selected methylation sites in the corresponding control cfDNA fragments. In some aspects, a binomial test is used to generate the statistical value for each of the test cfDNA fragments from the plurality of test cfDNA fragments.

In some aspects, the method further comprises generating one or more test cfDNA fragment metrics for the test cfDNA fragments labeled as originating from the tissue impacted by the disease. In some aspects, the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease. In some aspects, the method comprises inputting the one or more test cfDNA fragment metrics into a trained machine learning model configured to receive the one or more test cfDNA fragment metrics and predict whether the test subject has the disease.

In some aspects, the test subject is suspected of having or is determined to have the disease. In some aspects, the method is repeated for a plurality of different types of diseases using different trained filter models, wherein the different trained filter models are trained for the different types of diseases. In some aspects, the method is performed for a plurality of different types of cancers using different trained filter models, wherein the different trained filter models are trained for the different types of cancers. In some aspects, the method further comprises determining a most likely type of disease or most likely type of cancer for the test subject. In some aspects, the method is performed for a plurality of different types of cardiovascular or neurological diseases using different trained filter models, wherein the different trained filter models are trained for the different types of cardiovascular or neurological diseases. In some aspects, the method further comprises determining a most likely type of cardiovascular or neurological disease for the test subject.

In some aspects, disclosed herein is a method for diagnosing a disease, comprising: determining the most likely type of disease or the most likely type of cancer for the test subject according to any of the methods described above; and diagnosing the test subject as having the disease based on the determination of the most likely type of disease or the most likely type of cancer for the test subject. In some aspects, disclosed herein is a method of selecting an anti-cancer therapy, comprising: determining the most likely type of cancer for the test subject according to the preceding method; and responsive to the determination of the most likely type of cancer for the test subject, selecting an anti-cancer therapy for the test subject. In some aspects, disclosed herein is a method of treating a cancer in a subject, comprising: determining the most likely type of cancer for the test subject according to the preceding method; and responsive to determining the most likely type of cancer for the test subject, administering an effective amount of an anti-cancer therapy to the subject.

In some aspects, the anti-cancer therapy comprises a targeted anti-cancer therapy. In some aspects, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof. In some aspects, the anti-cancer therapy comprises resection, chemotherapy, radiation, or increased screening.

In some aspects, the trained filter model was previously trained according to a method comprising: (a) receiving, at one or more processors, sample sequencing data for a plurality of sample cfDNA fragments obtained from a training sample of a training subject having a disease, the sample sequencing data comprising, for each of one or more sample cfDNA fragments from the plurality of sample cfDNA fragments, a methylation status for one or more first selected methylation sites, a methylation fraction for one or more second selected methylation sites, and a fragment length; (b) generating, using the one or more processors, for each of the one or more sample cfDNA fragments, a value indicating that the sample cfDNA fragment originated from tissue not impacted by the disease based on the methylation status for the one or more first selected methylation sites in the sample cfDNA fragment and a methylation status for one or more third selected methylation sites in corresponding control cfDNA fragments; (c) determining, using the one or more processors, for each of the one or more sample cfDNA fragments, a methylation fraction difference between a methylation fraction for one or more fourth selected methylation sites in the corresponding control cfDNA fragments and the methylation fraction for the one or more second selected methylation sites in the sample cfDNA fragment; (d) determining, using the one or more processors, for each of the one or more sample cfDNA fragments, a fragment length difference between an average length of corresponding control cfDNA fragments and the fragment length of the sample cfDNA fragment; (e) determining, using the one or more processors, for each of a plurality of subgenomic intervals, a subgenomic interval-specific value threshold, a subgenomic interval-specific methylation fraction difference threshold, and a subgenomic interval-specific fragment length difference threshold based on the values, methylation fraction differences, and fragment length differences of each of the one or more sample cfDNA fragments that align with the subgenomic interval; and (f) setting, using the one or more processors, one or more thresholds of a filter model using the determined subgenomic interval-specific value thresholds, the determined subgenomic interval-specific methylation fraction difference thresholds, and the determined subgenomic interval-specific fragment length thresholds.

In some aspects, the method further comprises sequencing the sample cfDNA fragments to obtain the sample sequencing data. In some aspects, the sample sequencing data was obtained by: providing a plurality of sample cfDNA fragments obtained from a training sample from a training subject; ligating one or more adapters onto one or more sample cfDNA fragments from the plurality of sample cfDNA fragments; amplifying the one or more ligated sample cfDNA fragments from the plurality of sample cfDNA fragments; capturing amplified sample cfDNA fragments from the amplified sample cfDNA fragments; sequencing, by a sequencer, the captured sample cfDNA fragments to obtain a plurality of sequence reads that represent the captured sample cfDNA fragments. In some aspects, the captured sample cfDNA fragments are captured from the amplified sample cfDNA fragments by hybridization to one or more bait molecules. In some aspects, the one or more bait molecules comprise one or more cfDNA fragments, each comprising a region that is complementary to a region of a captured sample cfDNA fragment. In some aspects, the sequencing comprises use of a massively parallel sequencing (MPS) technique, whole genome sequencing (WGS), whole exome sequencing, targeted sequencing, direct sequencing, or Sanger sequencing technique. In some aspects, the sequencing comprises massively parallel sequencing, and the massively parallel sequencing technique comprises next generation sequencing (NGS).

In some aspects, the one or more first selected methylation sites, the one or more second selected methylation sites, the one or more third selected methylation sites, and the one or more fourth selected methylation sites are CpG sites in a reference sequence. In some aspects, the methylation status for the one or more first selected methylation sites comprises a number of methylated methylation sites and a number of unmethylated methylation sites, the methylation status for the one or more second selected methylation sites comprises a number of methylated methylation sites and a number of unmethylated methylation sites, the methylation status for the one or more third selected methylation sites comprises a number of methylated methylation sites and a number of unmethylated methylation sites, and the methylation status for the one or more fourth selected methylation sites comprises a number of methylated methylation sites and a number of unmethylated methylation sites.

In some aspects, the value is a p-value. In some aspects, generating the value comprises: aligning, using the one or more processors, the sample cfDNA fragment and the plurality of corresponding control cfDNA fragments to a reference sequence to generate an alignment, identifying from the alignment, using the one or more processors, corresponding control cfDNA fragments that positionally overlap with the sample cfDNA fragment, and comparing, using the one or more processors, the methylation status for the one or more first selected methylation sites in the sample cfDNA fragment to the methylation status for the one or more third selected methylation sites in the corresponding control cfDNA fragment. In some aspects, comparing the methylation status for the one or more first selected methylation sites in the sample cfDNA fragment to the methylation status for one or more third selected methylation sites in the corresponding control cfDNA fragments comprises determining a number of methylated sites in the one or more first selected methylation sites in the sample cfDNA fragment, a number of unmethylated sites in the one or more first selected methylation sites in the sample cfDNA fragment, a number of methylated sites in the one or more third selected methylation sites in the corresponding control cfDNA fragments, and a number of unmethylated sites in the one or more third selected methylation sites in the corresponding control cfDNA fragments. In some aspects, a binomial test is used to generate the value for each of the sample cfDNA fragments from the plurality of sample cfDNA fragments. In some aspects, determining the subgenomic interval-specific value threshold, the subgenomic interval-specific methylation fraction difference threshold, and the subgenomic interval-specific fragment length difference threshold comprises: determining a plurality of receiver operating characteristic (ROC) curves using a plurality of different subgenomic interval-specific value thresholds, a plurality of different subgenomic interval-specific methylation fraction difference thresholds, and a plurality of different subgenomic interval-specific fragment length difference thresholds; and selecting the subgenomic interval-specific value threshold, the subgenomic interval-specific methylation fraction difference threshold, and the subgenomic interval-specific fragment length difference threshold providing the highest area under the curve (AUC) among the plurality of ROC curves. In some aspects, the corresponding control cfDNA fragments were obtained from control subjects labeled as not having the disease.

In some aspects, the disease is a cancer. In some aspects, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some aspects, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some aspects, the method further comprises obtaining the test sample from the test subject or the training sample from the training subject. In some aspects, the test sample or the training sample comprises a tissue biopsy sample. In some aspects, the test sample or the training sample is a liquid biopsy sample and comprises blood, plasma, cerebrospinal fluid, sputum, stool, urine, or saliva. In some aspects, the cfDNA fragments or a portion thereof comprises circulating tumor DNA (ctDNA) fragments. In some aspects, the plurality of subgenomic intervals comprises between 10 and 20 subgenomic intervals, between 10 and 40 subgenomic intervals, between 10 and 60 subgenomic intervals, between 10 and 80 subgenomic intervals, between 10 and 100 subgenomic intervals, between 10 and 150 subgenomic intervals, between 10 and 200 subgenomic intervals, between 10 and 250 subgenomic intervals, between 10 and 300 subgenomic intervals, between 10 and 350 subgenomic intervals, between 10 and 400 subgenomic intervals, between 10 and 450 subgenomic intervals, between 10 and 500 subgenomic intervals, between 20 and 40 subgenomic intervals, between 20 and 60 subgenomic intervals, between 20 and 80 subgenomic intervals, between 20 and 100 subgenomic intervals, between 20 and 150 subgenomic intervals, between 20 and 200 subgenomic intervals, between 20 and 250 subgenomic intervals, between 20 and 300 subgenomic intervals, between 20 and 350 subgenomic intervals, between 20 and 400 subgenomic intervals, between 20 and 500 subgenomic intervals, between 40 and 60 subgenomic intervals, between 40 and 80 subgenomic intervals, between 40 and 100 subgenomic intervals, between 40 and 150 subgenomic intervals, between 40 and 200 subgenomic intervals, between 40 and 250 subgenomic intervals, between 40 and 300 subgenomic intervals, between 40 and 350 subgenomic intervals, between 40 and 400 subgenomic intervals, between 40 and 500 subgenomic intervals, between 60 and 80 subgenomic intervals, between 60 and 100 subgenomic intervals, between 60 and 150 subgenomic intervals, between 60 and 200 subgenomic intervals, between 60 and 250 subgenomic intervals, between 60 and 300 subgenomic intervals, between 60 and 350 subgenomic intervals, between 60 and 400 subgenomic intervals, between 60 and 500 subgenomic intervals, between 80 and 100 subgenomic intervals, between 80 and 150 subgenomic intervals, between 80 and 200 subgenomic intervals, between 80 and 250 subgenomic intervals, between 80 and 300 subgenomic intervals, between 80 and 350 subgenomic intervals, between 80 and 400 subgenomic intervals, between 80 and 500 subgenomic intervals, between 100 and 150 subgenomic intervals, between 100 and 200 subgenomic intervals, between 100 and 250 subgenomic intervals, between 100 and 300 subgenomic intervals, between 100 and 350 subgenomic intervals, between 100 and 400 subgenomic intervals, between 100 and 500 subgenomic intervals, between 150 and 200 subgenomic intervals, between 150 and 250 subgenomic intervals, between 150 and 300 subgenomic intervals, between 150 and 350 subgenomic intervals, between 150 and 400 subgenomic intervals, between 150 and 500 subgenomic intervals, between 200 and 250 subgenomic intervals, between 200 and 300 subgenomic intervals, between 200 and 350 subgenomic intervals, between 200 and 400 subgenomic intervals, between 200 and 500 subgenomic intervals, between 250 and 300 subgenomic intervals, between 250 and 350 subgenomic intervals, between 250 and 400 subgenomic intervals, between 250 and 500 subgenomic intervals, between 300 and 350 subgenomic intervals, between 300 and 400 subgenomic intervals, between 300 and 500 subgenomic intervals, between 350 and 400 subgenomic intervals, between 350 and 500 subgenomic intervals, or between 400 and 500 subgenomic intervals.

In some aspects, the method further comprises generating, by the one or more processors, a report indicating the determined subgenomic interval-specific value thresholds, the determined subgenomic interval-specific methylation fraction difference thresholds, the determined subgenomic interval-specific fragment length thresholds, a list of test cfDNA fragments labeled as originating from tissue impacted by the disease and cfDNA fragments, a list of test cfDNA fragments labeled as not originating from tissue impacted by the disease and cfDNA fragments, and/or one or more test cfDNA fragment metrics for the test cfNDA fragments. In some aspects, the method further comprises transmitting the report to a healthcare provider. In some aspects, the report is transmitted via a computer network or a peer-to-peer connection.

In some aspects, disclosed herein is a system comprising: one or more processors; and a memory communicatively coupled to the one or more processors and configured to store instructions that, when executed by the one or more processors, cause the system to: receive, at one or more processors, sample sequencing data for a plurality of sample cfDNA fragments; determine, using the one or more processors, for each of the one or more sample cfDNA fragments from the plurality of sample cfDNA fragments, at least one of: a value indicating that the sample cfDNA fragment originated from tissue not impacted by a disease; a methylation fraction difference between a methylation fraction for corresponding control cfDNA fragments and a methylation fraction for the sample cfDNA fragment; a fragment length difference between an average length of corresponding control cfDNA fragments and the fragment length of the sample cfDNA fragment; determine, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and set, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

In some aspects, the plurality of sample cfDNA fragments are obtained from a training sample of a training subject having a disease. In some aspects, the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length. In some aspects, the value is a statistical value indicating the probability that the sample cfDNA fragment originated from tissue not impacted by a disease, and the statistical value is based on the methylation status for the one or more first selected methylation sites in the sample cfDNA fragment and a methylation status for one or more third selected methylation sites in corresponding control cfDNA fragments. In some aspects, the methylation fraction difference is between the methylation fraction for one or more fourth selected methylation sites in the corresponding control cfDNA fragments and the methylation fraction for one or more second selected methylation sites in the sample cfDNA fragment.

In some aspects, the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment. In some aspects, the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment.

In some aspects, the plurality of test cfDNA fragments are obtained from a test sample of a test subject. In some aspects, the selected corresponding control cfDNA fragments are selected based on alignment within a common subgenomic interval with the test cfDNA fragment. In some aspects, the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length. In some aspects, the value is a statistical value indicating the probability that the sample cfDNA fragment originated from tissue not impacted by a disease, and the statistical value is based on the methylation status for the one or more first selected methylation sites in the test cfDNA fragment and a methylation status for one or more third selected methylation sites in corresponding control cfDNA fragments. In some aspects, the methylation fraction difference is between the methylation fraction for one or more fourth selected methylation sites in the corresponding control cfDNA fragments and the methylation fraction for one or more second selected methylation sites in the test cfDNA fragment.

In some aspects, the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment. In some aspects, the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment.

In some aspects, the instructions, when executed by the one or more processors, further cause the system to generate one or more test cfDNA fragment metrics for the test cfDNA fragments labeled as originating from the tissue impacted by the disease. In some aspects, the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease.

In some aspects, the instructions, when executed by the one or more processors, further cause the system to input the one or more test cfDNA fragment metrics into a trained machine learning model configured to receive the one or more test cfDNA fragment metrics and predict whether the test subject has the disease. In some aspects, the instructions, when executed by the one or more processors, further cause the system to label, using the one or more processors, the test cfDNA fragments for a plurality of different types of diseases or different types of cancers using different trained filter models, wherein the different trained filter models are trained for the different types of diseases or different types of cancers. In some aspects, the instructions, when executed by the one or more processors, further cause the system to determine a most likely type of disease or most likely type of cancer for the test subject. In some aspects, the trained filter model was previously trained using a system as described above. In some aspects, the system further comprises a sequencer.

In some aspects, disclosed herein is a non-transitory computer-readable storage medium storing one or more programs, the one or more programs comprising instructions, which when executed by one or more processors of a system, cause the system to: receive, at one or more processors, sample sequencing data for a plurality of sample cfDNA fragments; determine, using the one or more processors, for each of one or more sample cfDNA fragments from the plurality of sample cfDNA fragments, at least one of: a value indicating that the sample cfDNA fragment originated from tissue not impacted by a disease; a methylation fraction difference between a methylation fraction for corresponding control cfDNA fragments and a methylation fraction for the sample cfDNA fragment, and a fragment length difference between an average length of corresponding control cfDNA fragments and a fragment length of the sample cfDNA fragment; determine, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and set, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

In some aspects, the plurality of sample cfDNA fragments are obtained from a training sample of a training subject having a disease. In some aspects, the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length. In some aspects, the value is a statistical value indicating the probability that the sample cfDNA fragment originated from tissue not impacted by a disease, and the statistical value is based on the methylation status for the one or more first selected methylation sites in the sample cfDNA fragment and a methylation status for one or more third selected methylation sites in corresponding control cfDNA fragments. In some aspects, the methylation fraction difference is between the methylation fraction for one or more fourth selected methylation sites in the corresponding control cfDNA fragments and the methylation fraction for one or more second selected methylation sites in the sample cfDNA fragment.

In some aspects, the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment. In some aspects, the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment.

In some aspects, disclosed herein is a non-transitory computer-readable storage medium storing one or more programs, the one or more programs comprising instructions, which when executed by one or more processors of a system, cause the system to: receive, at one or more processors, test sequencing data for a plurality of test cfDNA fragments; determine, using the one or more processors, for each of one or more test cfDNA fragments from the plurality of test cfDNA fragments, at least one of: a value indicating that the test cfDNA fragment originated from tissue not impacted by the disease; a methylation fraction difference between a methylation fraction for corresponding control cfDNA fragments and a methylation fraction for the test cfDNA fragment; a fragment length difference between an average length of corresponding control cfDNA fragments and a fragment length of the test cfDNA fragment; input, using the one or more processors, for each of the one or more test cfDNA fragments, at least one of the value, the methylation fraction difference, and the fragment length difference into a trained filter model; filter, using the one or more processors, the plurality of test cfDNA fragments using the filter model; and label, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

In some aspects, the plurality of test cfDNA fragments are obtained from a test sample of a test subject. In some aspects, the selected corresponding control cfDNA fragments are selected based on alignment within a common subgenomic interval with the test cfDNA fragment. In some aspects, the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length. In some aspects, the value is a statistical value indicating the probability that the sample cfDNA fragment originated from tissue not impacted by a disease, and the statistical value is based on the methylation status for the one or more first selected methylation sites in the test cfDNA fragment and a methylation status for one or more third selected methylation sites in corresponding control cfDNA fragments. In some aspects, the methylation fraction difference is between the methylation fraction for one or more fourth selected methylation sites in the corresponding control cfDNA fragments and the methylation fraction for one or more second selected methylation sites in the test cfDNA fragment.

In some aspects, the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment. In some aspects, the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment. In some aspects, the instructions, when executed by the one or more processors, further cause the system to generate one or more test cfDNA fragment metrics for the test cfDNA fragments labeled as originating from the tissue impacted by the disease. In some aspects, the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIGS. 1A-1D provide non-limiting schematic illustrations of how features may be calculated to describe sample or test cfDNA fragments compared to corresponding control cfDNA fragments.

FIG. 1A depicts how the methylation status for methylation sites (“M” or “U”) may be compared between a sample or test cfDNA fragment and corresponding control cfDNA fragments, where the entirety of a sample or test cfDNA fragment aligns or overlaps with one subgenomic interval (dotted lines). FIG. 1B depicts how the methylation status for methylation sites (“M” or “U”) may be compared between a sample or test cfDNA fragment and corresponding control cfDNA fragments, where a sample or test cfDNA fragment aligns or overlaps with more than one subgenomic interval (dotted lines). FIG. 1C provides depicts how the fragment length of a sample or test cfDNA fragment may be compared to the average fragment length of corresponding control cfDNA fragments, where the entirety of a sample or test cfDNA fragment aligns or overlaps with one subgenomic interval (dotted lines) and the entirety of corresponding control cfDNA fragments aligns or overlaps with the same subgenomic interval. FIG. 1D depicts how the fragment length of a sample or test cfDNA fragment may be compared to the average fragment length of corresponding control cfDNA fragments, where a sample or test cfDNA fragment aligns or overlaps with more than one subgenomic interval (dotted lines), and the corresponding control cfDNA fragments align or overlap with more than one subgenomic interval.

FIG. 2 provides a non-limiting example of a process for training a filter model for filtering sequencing data for cfDNA fragments, according to some embodiments disclosed herein.

FIG. 3 provides another non-limiting example of a process for training a filter model for filtering sequencing data cfDNA fragments using at least one of three features, according to some embodiments disclosed herein.

FIG. 4 provides a non-limiting example of a process for labeling test cfDNA fragments from a test subject, according to some embodiments disclosed herein.

FIG. 5 provides another non-limiting example of a process for labeling test cfDNA fragments from a test subject using at least one of three features, according to some embodiments disclosed herein.

FIG. 6 provides a non-limiting example of a process for generating test cfDNA fragment metrics for test cfDNA fragments labeled as originating from tissue impacted by disease, according to some embodiments disclosed herein.

FIG. 7 provides another non-limiting example of a process for generating cfDNA fragment metrics.

FIG. 8 depicts an exemplary computing device or system in accordance with one embodiment of the present disclosure.

FIG. 9 depicts an exemplary computer system or computer network, in accordance with some instances of the systems described herein.

FIGS. 10A-10C provide non-limiting examples of feature distributions for sample cfDNA fragments aligning with one subgenomic interval. Each dot represents data from a cfDNA fragment. FIG. 10A depicts the methylation fraction difference values (“mF”) compared against the fragment length values (“tlen”) for the sample cfDNA fragments. FIG. 10B depicts the p-values (“p value”) compared against the methylation fraction differences (“mF_delta”) for the sample cfDNA fragments. FIG. 10C depicts the p-values (“p value”) compared against the fragment length differences (“tlen_delta”) for the sample cfDNA fragments.

FIG. 11 provides a non-limiting example of the fraction of sample cfDNA fragments that pass through set subgenomic interval-specific thresholds compared to the fraction of control cfDNA fragments that pass through set subgenomic interval-specific thresholds.

### DETAILED DESCRIPTION

Methods and systems for training a filter model for filtering sequencing data for cfDNA fragments and labeling test cfDNA fragments as originating from tissue impacted by disease are described. Features of cfDNA can be used to detect disease in subjects using a minimally invasive sample. However, samples often contain a majority of cfDNA fragments derived from healthy cells or tissue not associated with disease. It is difficult to identify cfDNA fragments and features of cfDNA fragments associated with diseased tissue or healthy tissue impacted by disease, distinct from cfDNA fragments that likely originate from healthy tissue not impacted by disease. These issues can lead to challenges in assay sensitivity for early-stage detection of disease and diminished accuracy in tissue of origin detection. Existing approaches have focused on analyzing singular features associated with cfDNA fragments as associated with disease, for example, only the methylation status of cfDNA fragments. This can be error-prone and limited by sample variability. There is a need to develop methods and systems that can accurately distinguish between cfDNA that originated from cells or tissue not impacted by disease and cfDNA that originated from cells or tissue impacted by disease. Provided herein are methods and systems for filtering sequencing data for cfDNA fragments associated with disease and labeling test cfDNA fragments as originating from tissue impacted by disease. The disclosed methods are based at least partially on insight that cfDNA fragments and features of cfDNA fragments are likely to vary considerably between biological contexts, which may be examined in different genomic regions or subgenomic intervals.

In some instances, for example, a method of training a filter model for filtering sequencing data for cfDNA fragments is described, comprising receiving sample sequencing data for a plurality of sample cfDNA fragments obtained from a training sample of a training subject having a disease, the sample sequencing data comprising, for each of one or more sample cfDNA fragments from the plurality of sample cfDNA fragments, a methylation status for one or more first selected methylation sites, a methylation fraction for one or more second selected methylation sites, and a fragment length; generating, using the one or more processors, for each of the one or more sample cfDNA fragments, a value that the sample cfDNA fragment originated from tissue not impacted by the disease based on the methylation status for the one or more first selected methylation sites in the sample cfDNA fragment and a methylation status for one or more third selected methylation sites in corresponding control cfDNA fragments; determining, using the one or more processors, for each of the one or more sample cfDNA fragments, a methylation fraction difference between a methylation fraction for one or more fourth selected methylation sites in the corresponding control cfDNA fragments and the methylation fraction for the one or more second selected methylation sites in the sample cfDNA fragment; determining, using the one or more processors, for each of the one or more sample cfDNA fragments, a fragment length difference between an average length of selected corresponding control cfDNA fragments and the fragment length of the sample cfDNA fragment, wherein the selected corresponding control cfDNA fragments are selected based on alignment within a common subgenomic interval with the sample cfDNA fragment; determining, using the one or more processors, for each of a plurality of subgenomic intervals, a subgenomic interval-specific value threshold, a subgenomic interval-specific methylation fraction difference threshold, and a subgenomic interval-specific fragment length difference threshold based on the values, methylation fraction differences, and fragment length differences of each of the one or more sample cfDNA fragments that align with the subgenomic interval; and setting, using the one or more processors, one or more thresholds of a filter model using the determined subgenomic interval-specific value thresholds, the determined subgenomic interval-specific methylation fraction difference thresholds, and the determined subgenomic interval-specific fragment length thresholds.

In some instances, for example, a method of labeling test cfDNA fragments from a test subject is described, comprising receiving, at one or more processors, test sequencing data comprising, for each of one or more test cfDNA fragments from a plurality of test cfDNA fragments from a test sample from the test subject, a methylation status for one or more first selected methylation sites, a methylation fraction for one or more second selected methylation sites, and a fragment length; generating, using the one or more processors, for each of the one or more test cfDNA fragments, a value indicating that the test cfDNA fragment originated from tissue not impacted by the disease based on the methylation status for the one or more first selected methylation sites in the test cfDNA fragment and a methylation status for one or more third selected methylation sites in corresponding control cfDNA fragments; determining, using the one or more processors, for each of the one or more test cfDNA fragments, a methylation fraction difference between a methylation fraction for one or more fourth selected methylation sites in the corresponding control cfDNA fragments and the methylation fraction for the one or more second selected methylation sites in the test cfDNA fragment; determining, using the one or more processors, for each of the one or more test cfDNA fragments, a fragment length difference between an average length of selected corresponding control cfDNA fragments and the fragment length of the test cfDNA fragment, wherein the selected corresponding control cfDNA fragments are selected based on alignment within a common subgenomic interval with the test cfDNA fragment; inputting, using the one or more processors, for each of the one or more test cfDNA fragments, the value, the methylation fraction difference, and the fragment length difference into a trained filter model; filtering, using the one or more processors, the plurality of test cfDNA fragments using the filter model; and labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

In some instances, the test sample or the training sample is a liquid biopsy sample and comprises blood, plasma, cerebrospinal fluid, sputum, stool, urine, or saliva. In some instances, the method of labeling test cfDNA fragments from a test subject further comprises generating one or more test cfDNA fragment metrics for the test cfDNA fragments labeled as originating from the tissue impacted by the disease.

### DEFINITIONS

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “sequence read” is a computationally generated sequence generated by a sequencer to represent a sequence of bases of a single strand of a sequenced fragment. A sequence read can refer to a raw sequence read (e.g., a sequence read as obtained directly from a sequencing instrument), an aligned sequence read (e.g., a sequence read that has been aligned to a reference genome), a single-end sequence read, a paired-end sequence read, a merged sequence read (e.g., a sequence read based on merging a group of overlapping paired-end reads), a consensus sequence read (e.g., a sequence read based on performing error correction on a merged sequence read), a computationally reconstructed sequence read (e.g., a sequence read that has been computationally truncated at the 5′ and/or 3′ end), or any combination thereof.

As used herein, the terms “variant sequence” or “variant” are used interchangeably and refer to a modified nucleic acid sequence relative to a corresponding “normal” or “wild-type” sequence.

In some instances, a variant sequence may be a “short variant sequence” (or “short variant”), i.e., a variant sequence of less than about 50 base pairs in length.

As used herein, the term “feature” refers to a parameter, attribute, statistic, value, or metric that may be generated in relation to cfDNA fragments and subsequently used in a model.

As used herein, “each of” may refer to a subset of a totality or the entirety of the totality. For example, “each of the one or more sample cfDNA fragments from the plurality of sample cfDNA fragments” can refer to a subset of the plurality of sample cfDNA fragments and not the totality of the plurality of sample cfDNA fragments.

As used herein, “corresponding control cfDNA fragments” refers to control cfDNA fragments that correspond to a given sample cfDNA fragment or to a given test cfDNA fragment, where “correspond” refers to control cfDNA fragments that align with or overlap with a sample cfDNA fragment or a test cfDNA fragment, or to control cfDNA fragments that align with the same subgenomic interval as a sample cfDNA fragment or a test cfDNA fragment.

The section headings used herein are for organizational purposes only and are not to be construed as limiting the subject matter described.

### Methods of Calculating Model Features

The methods disclosed herein involve analyzing sequencing data for cfDNA fragments and using the data to identify cfDNA fragments that are likely to have originated from tissue impacted by disease or not. These methods may include calculating features of cfDNA fragments that biologically describe the cfDNA fragments. Features may be calculated for cfDNA fragments using computer-implemented processes.

For example, a cfDNA fragment may contain one or more methylation sites, which may be unmethylated or methylated. Information about the number of methylation sites that are unmethylated or methylated in a cfDNA fragment may be compared between different types of cfDNA fragments. Information about the methylation sites may be used to determine a methylation status for methylation sites in a cfDNA fragment. Methylation statuses may be compared between cfDNA fragments to generate a value for a cfDNA fragment which can be a statistical value. Information about the methylation sites may also be used to determine a methylation fraction for methylation sites in a cfDNA fragment. Methylation fractions may be compared between cfDNA fragments to generate a methylation fraction difference for a cfDNA fragment. Different cfDNA fragments may have different lengths. Information about the length of a cfDNA fragment may also be compared between different types of cfDNA fragments to determine a fragment length difference for a cfDNA fragment.

In the methods disclosed herein, sequencing data for cfDNA fragments may be obtained from a training sample of a training subject having a disease, wherein the sequencing data comprises a plurality of cfDNA fragments referred to as sample cfDNA fragments. Alternatively, sequencing data for cfDNA fragments may be obtained from a test sample from a test subject, wherein the sequencing data comprises a plurality of cfDNA fragments referred to as test cfDNA fragments. Alternatively, sequencing data for cfDNA fragments may be obtained from a control sample of a control subject labeled as not having a disease, wherein the sequencing data comprises a plurality of cfDNA fragments referred to as control cfDNA fragments.

As described above, features may be calculated for these different types of cfDNA fragments. For example, in some aspects, features are calculated for sample cfDNA fragments compared to control cfDNA fragments. In some aspects, features are calculated for test cfDNA fragments compared to control cfDNA fragments. Sample or test cfDNA fragments can be compared to corresponding control cfDNA fragments. Corresponding control cfDNA fragments are those that align with or overlap with a sample cfDNA fragment or a test cfDNA fragment by alignment to the same genomic position, or those that align with the same subgenomic interval as a sample cfDNA fragment or a test cfDNA fragment.

Features can be calculated for sample or test cfDNA fragments compared to corresponding control cfDNA fragments in subgenomic intervals. Each sample or test cfDNA fragment in a subgenomic interval can have its own feature or set of features. For comparing sample or test cfDNA fragments to corresponding control cfDNA fragments, a sample or test cfDNA fragment and a plurality of corresponding control cfDNA fragments can be aligned to a reference sequence to generate an alignment. Sample or test cfDNA fragments can be compared with the corresponding control cfDNA fragments to which the sample or test cfDNA fragments positionally overlap, as described below in the context of calculating different types of features. The positional overlaps between sample or test cfDNA fragments and corresponding control cfDNA fragments can occur within different subgenomic intervals, determined by the alignment of all the cfDNA fragments to the reference sequence.

A given sample cfDNA fragment in a plurality of sample cfDNA fragments may have its own feature as compared to corresponding control cfDNA fragments. Alternatively, each of a subset of sample cfDNA fragments in a plurality of sample cfDNA fragments may have its own feature as compared to corresponding control cfDNA fragments. Each test cfDNA fragment in a plurality of test cfDNA fragments may have its own feature as compared to corresponding control cfDNA fragments. Alternatively, each of a subset of test cfDNA fragments in a plurality of test cfDNA fragments may have its own feature as compared to corresponding control cfDNA fragments.

### Value

In some aspects, a feature that may be calculated for cfDNA fragments is a value that indicates that a cfDNA fragment originated from tissue not impacted by disease. The value can be a statistical value that indicates a probability that a cfDNA fragment originated from tissue not impacted by disease. The statistical value compares the methylation status for selected methylation sites within cfDNA fragments to the methylation status for selected methylation sites within other cfDNA fragments. For example, a statistical value may be calculated for a sample cfDNA fragment compared to corresponding control cfDNA fragments, comparing the methylation statuses of those different types of fragments. Alternatively, a statistical value may be calculated for a test cfDNA fragment compared to corresponding control cfDNA fragments, comparing the methylation statuses of those different types of fragments.

The calculation of a statistical value for a sample cfDNA fragment or a test cfDNA fragment compared to corresponding control cfDNA fragments involves a determination of methylation statuses. The methylation status of a nucleic acid fragment refers to a binary status (e.g., methylated or non-methylated) of a nucleic acid at one or more positions within the nucleic acid fragment. The methylation status may be a status indicating the entirety of the nucleic acid fragment, which can be represented, for example, as a one-dimensional vector describing the methylation pattern in the fragment for a collection of selected methylation sites, and may include a number of methylated methylation sites, a number of unmethylated methylation sites, or a number of methylated methylation sites and a number of unmethylated methylation sites.

In some aspects of the present invention, a sample cfDNA fragment may have one methylation status that describes all the unmethylated or methylated methylation sites within the sample cfDNA fragment. In some aspects, a sample cfDNA fragment may have one methylation status that describes only selected unmethylated or methylated methylation sites within the sample cfDNA fragment. In some aspects, a test cfDNA fragment may have one methylation status that describes all the unmethylated or methylated methylation sites within the test cfDNA fragment. In some aspects, a test cfDNA fragment may have one methylation status that describes only selected unmethylated or methylated methylation sites within the test cfDNA fragment. In some aspects, corresponding control cfDNA fragments may have one methylation status that describes all the unmethylated or methylated methylation sites within all the corresponding control cfDNA fragments. In some aspects, corresponding control cfDNA fragments may have one methylation status that describes only selected unmethylated or methylated methylation sites within all the corresponding control cfDNA fragments.

For calculating a statistical value, the methylation status for selected methylation sites in a cfDNA fragment is compared to the methylation status for selected methylation sites in other cfDNA fragments. For example, the methylation status for selected methylation sites in a sample cfDNA fragment can be compared to the methylation status for selected methylation sites in corresponding control cfDNA fragments. In such an example, the selected methylation sites in the sample cfDNA fragment may be referred to as the first selected methylation sites and the selected methylation sites in corresponding control cfDNA fragments may be referred to as third selected methylation sites. Alternatively, the methylation status for selected methylation sites in a test cfDNA fragment can be compared to the methylation status for selected methylation sites in corresponding control cfDNA fragments. In such an example, the selected methylation sites in the test cfDNA fragment may be referred to as the first selected methylation sites and the selected methylation sites in corresponding control cfDNA fragments may be referred to as third selected methylation sites. In both cases, the corresponding control cfDNA fragments are the control cfDNA fragments that align with or overlap with a sample cfDNA fragment or a test cfDNA fragment by alignment to the same genomic position, or the control cfDNA fragments that align with the same subgenomic interval as a sample cfDNA fragment or a test cfDNA fragment.

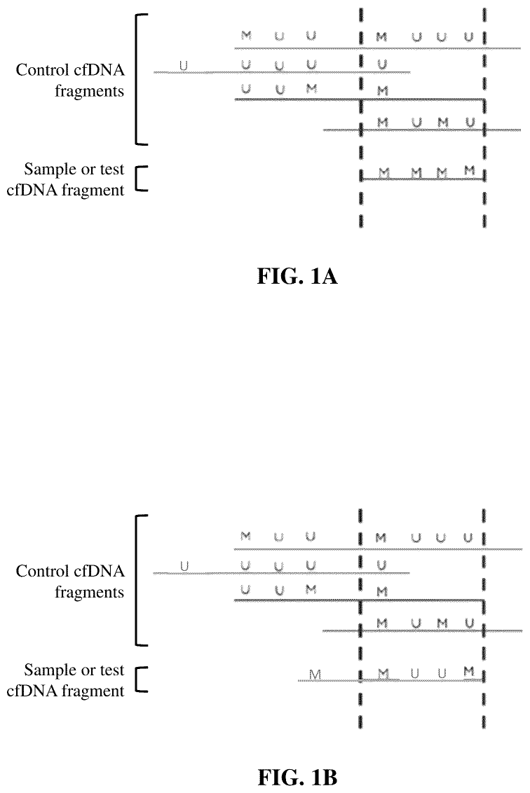

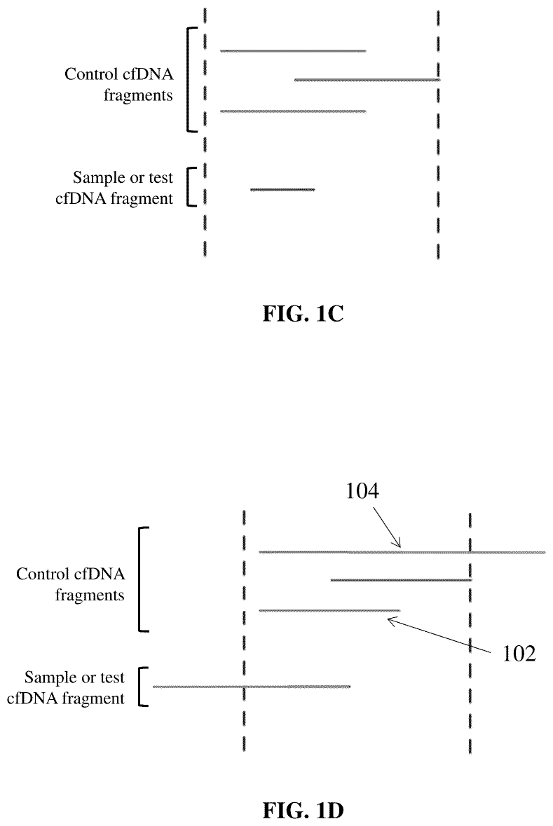

With reference to FIG. 1A, for example, the methylation status for selected methylation sites of a sample or test cfDNA fragment may be 4 methylated methylation sites and 0 unmethylated methylation sites, where the selected methylation sites are all the methylation sites in the sample or test cfDNA fragment. Alternatively, the methylation status for selected methylation sites of a sample or test cfDNA fragment may be 4 methylated methylation sites and 0 unmethylated methylation sites, where the selected methylation sites are only the methylation sites in the sample or test cfDNA fragment that positionally overlap with selected methylation sites in corresponding control cfDNA fragments that align within a common subgenomic interval with the sample or test cfDNA fragment. In such an example, the corresponding control cfDNA fragments are the control cfDNA fragments that align or overlap, in any part, within the same subgenomic interval (the dotted lines in FIG. 1A) as the sample or test cfDNA fragment (i.e., only part of the control cfDNA fragment aligns within the same subgenomic interval to correspond), the selected methylation sites in the corresponding control cfDNA fragments are the methylation sites that align or overlap with the same subgenomic interval as the sample or test cfDNA fragment, and the selected methylation sites in the sample or test cfDNA fragment are the methylation sites that align with the selected methylation sites (“M” or “U” in FIG. 1A) in the corresponding control cfDNA fragments.

With reference to FIG. 1A, for example, the methylation status for selected methylation sites in corresponding control cfDNA fragments may be 6 methylated methylation sites and 14 unmethylated methylation sites, where the selected methylation sites are all the methylation sites in the corresponding control cfDNA fragments. Alternatively, the methylation status for selected methylation sites in corresponding control cfDNA fragments may be 4 methylated methylation sites and 6 unmethylated methylation sites, where the selected methylation sites are only the methylation sites in the corresponding control cfDNA fragments that positionally overlap with selected methylation sites in a sample or test cfDNA fragment (to which the corresponding control cfDNA fragments correspond) and that align within a common subgenomic interval with the sample or test cfDNA fragment. In such an example, the corresponding control cfDNA fragments are the control cfDNA fragments that align or overlap, in any part, within the same subgenomic interval (the dotted lines in FIG. 1A) as the sample or test cfDNA fragment (i.e., only part of the control cfDNA fragment aligns within the same subgenomic interval to correspond), the selected methylation sites in the sample or test cfDNA fragment (“M” or “U” in FIG. 1A) are the methylation sites that align or overlap with the same subgenomic interval (the dotted lines in FIG. 1A) as the corresponding control cfDNA fragments, and the selected methylation sites in the corresponding control cfDNA fragments are the methylation sites that align with the selected methylation sites in the sample or test cfDNA fragment.

With reference to FIG. 1B, for example, the methylation status for selected methylation sites of a sample or test cfDNA fragment may be 3 methylated methylation sites and 2 unmethylated methylation sites, where the selected methylation sites are all the methylation sites in the sample or test cfDNA fragment. Alternatively, the methylation status for selected methylation sites of a sample or test cfDNA fragment may be 2 methylated methylation sites and 2 unmethylated methylation sites, where the selected methylation sites are only the methylation sites in the sample or test cfDNA fragment that positionally overlap with selected methylation sites in corresponding control cfDNA fragments that align within a common subgenomic interval with the sample or test cfDNA fragment. In such an example, the corresponding control cfDNA fragments are the control cfDNA fragments that align or overlap, in any part, within the same subgenomic interval (the dotted lines in FIG. 1B) as the sample or test cfDNA fragment (i.e., only part of the control cfDNA fragment aligns within the same subgenomic interval to correspond), the selected methylation sites in the corresponding control cfDNA fragments are the methylation sites that align or overlap with the same subgenomic interval as the sample or test cfDNA fragment, and the selected methylation sites in the sample or test cfDNA fragment are the methylation sites that align with the selected methylation sites (“M” or “U” in FIG. 1B) in the corresponding control cfDNA fragments.

With reference to FIG. 1B, for example, the methylation status for selected methylation sites in corresponding control cfDNA fragments may be 6 methylated methylation sites and 14 unmethylated methylation sites, where the selected methylation sites are all the methylation sites in the corresponding control cfDNA fragments. Alternatively, the methylation status for selected methylation sites in corresponding control cfDNA fragments may be 4 methylated methylation sites and 6 unmethylated methylation sites, where the selected methylation sites are only the methylation sites in the corresponding control cfDNA fragments that positionally overlap with selected methylation sites in a sample or test cfDNA fragment (to which the corresponding control cfDNA fragments correspond) and that align within a common subgenomic interval with the sample or test cfDNA fragment. In such an example, the corresponding control cfDNA fragments are the control cfDNA fragments that align or overlap, in any part, within the same subgenomic interval (the dotted lines in FIG. 1B) as the sample or test cfDNA fragment, the selected methylation sites in the sample or test cfDNA fragment (“M” or “U” in FIG. 1B) are the methylation sites that align or overlap with the same subgenomic interval (the dotted lines in FIG. 1B) as the corresponding control cfDNA fragments, and the selected methylation sites in the corresponding control cfDNA fragments are the methylation sites that align with the selected methylation sites in the sample or test cfDNA fragment.

The methylation statuses for selected methylation sites, in any of the instances described above, may be compared between sample or test cfDNA fragments and corresponding control cfDNA fragments to generate statistical values. In some aspects of the present invention, the statistical value is a p-value derived from a binomial test that compares the methylation statuses between sample or test cfDNA fragments and corresponding control cfDNA fragments. In some aspects of the present invention, the statistical value is a Kullback-Leibler divergence score or a value determined by a Chi-squared test or Fisher's exact test that compares the methylation statuses between sample or test cfDNA fragments and corresponding control cfDNA fragments.

### Methylation Fraction Difference

In some aspects, a feature that may be calculated for cfDNA fragments is a methylation fraction difference between different types of cfDNA fragments. The methylation fraction difference compares the methylation fraction for selected methylation sites within cfDNA fragments to the methylation fraction for selected methylation sites within other cfDNA fragments. For example, a methylation fraction difference may be calculated for a sample cfDNA fragment compared to corresponding control cfDNA fragments, comparing the methylation fractions of those different types of fragments. Alternatively, a methylation fraction difference may be calculated for a test cfDNA fragment compared to corresponding control cfDNA fragments, comparing the methylation fractions of those different types of fragments.

The calculation of a methylation fraction difference for a sample cfDNA fragment or a test cfDNA fragment compared to corresponding control cfDNA fragments involves a determination of methylation fractions. The methylation fraction of a nucleic acid fragment may refer to a fraction determined for the total number of positions within the entirety of the nucleic acid fragment that are methylated compared to the total number of positions within the entirety of the nucleic acid fragment that are methylated or unmethylated. Alternatively, the methylation fraction of a nucleic acid fragment may refer to a fraction determined for a selected number of positions within the nucleic acid fragment that are methylated compared to the total selected number of positions within the nucleic acid fragment that are methylated or unmethylated.

In some aspects of the present invention, a sample cfDNA fragment may have one methylation fraction that describes all the unmethylated or methylated methylation sites within the sample cfDNA fragment. In some aspects, a sample cfDNA fragment may have one methylation fraction that describes only selected unmethylated or methylated methylation sites within the sample cfDNA fragment. In some aspects, a test cfDNA fragment may have one methylation fraction that describes all the unmethylated or methylated methylation sites within the test cfDNA fragment. In some aspects, a test cfDNA fragment may have one methylation fraction that describes only selected unmethylated or methylated methylation sites within the test cfDNA fragment. In some aspects, corresponding control cfDNA fragments may have one methylation fraction that describes all the unmethylated or methylated methylation sites within all the corresponding control cfDNA fragments. In some aspects, corresponding control cfDNA fragments may have one methylation fraction that describes only selected unmethylated or methylated methylation sites within all the corresponding control cfDNA fragments.

For calculating a methylation fraction difference, the methylation fraction for selected methylation sites in a cfDNA fragment is compared to the methylation fraction for selected methylation sites in other cfDNA fragments. For example, the methylation fraction for selected methylation sites in a sample cfDNA fragment can be compared to the methylation fraction for selected methylation sites in corresponding control cfDNA fragments. In such an example, the selected methylation sites in the sample cfDNA fragment may be referred to as the second selected methylation sites and the selected methylation sites in corresponding control cfDNA fragments may be referred to as fourth selected methylation sites. Alternatively, the methylation fraction for selected methylation sites in a test cfDNA fragment can be compared to the methylation fraction for selected methylation sites in corresponding control cfDNA fragments. In such an example, the selected methylation sites in the test cfDNA fragment may be referred to as the second selected methylation sites and the selected methylation sites in corresponding control cfDNA fragments may be referred to as fourth selected methylation sites. In both cases, the corresponding control cfDNA fragments are the control cfDNA fragments that align with or overlap with a sample cfDNA fragment or a test cfDNA fragment by alignment to the same genomic position, or the control cfDNA fragments that align with the same subgenomic interval as a sample cfDNA fragment or a test cfDNA fragment.

With reference to FIG. 1A, for example, the methylation fraction for selected methylation sites of a sample or test cfDNA fragment is 1 if the methylation sites are selected as all the methylation sites in the sample or test cfDNA fragment, and 4 are methylated and 0 are unmethylated (4 methylation sites in total). Alternatively, the methylation fraction for selected methylation sites of a sample or test cfDNA fragment is 1 if the methylation sites are selected as only the methylation sites that positionally overlap with methylation sites in corresponding control cfDNA fragment that align within the same subgenomic interval as the sample or test cfDNA fragment, and 4 are methylated and 0 are unmethylated (4 methylation sites in total). In such an example, the corresponding control cfDNA fragments are the control cfDNA fragments that align or overlap, in any part, within the same subgenomic interval (the dotted lines in FIG. 1A) as the sample or test cfDNA fragment (i.e., only part of the control cfDNA fragment aligns within the same subgenomic interval to correspond), the selected methylation sites in the corresponding control cfDNA fragments are the methylation sites that align or overlap with the same subgenomic interval as the sample or test cfDNA fragment, and the selected methylation sites in the sample or test cfDNA fragment are the methylation sites that align with the selected methylation sites (“M” or “U” in FIG. 1A) in the corresponding control cfDNA fragments.

With reference to FIG. 1A, for example, the methylation fraction for selected methylation sites in corresponding control cfDNA fragments is 0.3 if the methylation sites are selected as all the methylation sites in the corresponding control cfDNA fragments, and 6 are methylated and 14 are unmethylated (20 methylation sites in total). Alternatively, the methylation fraction for selected methylation sites in corresponding control cfDNA fragments is 0.4 if the methylation sites are selected as only the methylation sites that positionally overlap with selected methylation sites in a sample or test cfDNA fragment (to which the corresponding control cfDNA fragments correspond) and align within a common subgenomic interval with the sample or test cfDNA fragment, and 4 are methylated and 6 are unmethylated (10 methylation sites in total). In such an example, the corresponding control cfDNA fragments are the control cfDNA fragments that align or overlap, in any part, within the same subgenomic interval (the dotted lines in FIG. 1A) as the sample or test cfDNA fragment (i.e., only part of the control cfDNA fragment aligns within the same subgenomic interval to correspond), the selected methylation sites in the sample or test cfDNA fragment (“M” or “U” in FIG. 1A) are the methylation sites that align or overlap with the same subgenomic interval (the dotted lines in FIG. 1A) as the corresponding control cfDNA fragments, and the selected methylation sites in the corresponding control cfDNA fragments are the methylation sites that align with the selected methylation sites in the sample or test cfDNA fragment.

With reference to FIG. 1B, for example, the methylation fraction for selected methylation sites of a sample or test cfDNA fragment is 0.6 if the methylation sites are selected as all the methylation sites in the sample or test cfDNA fragment, and 3 are methylated and 2 are unmethylated (5 methylation sites in total). Alternatively, the methylation fraction for selected methylation sites of a sample or test cfDNA fragment is 0.5 if the methylation sites are selected as only the methylation sites that positionally overlap with selected methylation sites in corresponding control cfDNA fragments that align within the same subgenomic interval as the sample or test cfDNA fragment, and 2 are methylated and 2 are unmethylated (4 methylation sites in total). In such an example, the corresponding control cfDNA fragments are the control cfDNA fragments that align or overlap, in any part, within the same subgenomic interval (the dotted lines in FIG. 1B) as the sample or test cfDNA fragment (i.e., only part of the control cfDNA fragment aligns within the same subgenomic interval to correspond), the selected methylation sites in the corresponding control cfDNA fragments are the methylation sites that align or overlap with the same subgenomic interval as the sample or test cfDNA fragment, and the selected methylation sites in the sample or test cfDNA fragment are the methylation sites that align with the selected methylation sites (“M” or “U” in FIG. 1B) in the corresponding control cfDNA fragments.

With reference to FIG. 1B, for example, the methylation fraction for selected methylation sites in corresponding control cfDNA fragments is 0.3 if the methylation sites are selected as all the methylation sites in the corresponding control cfDNA fragments, and 6 are methylated and 14 are unmethylated (20 methylation sites in total). Alternatively, the methylation fraction for selected methylation sites in corresponding control cfDNA fragments is 0.4 if the methylation sites are selected as only the methylation sites that positionally overlap with selected methylation sites in a sample or test cfDNA fragment (to which the corresponding control cfDNA fragments correspond) and align within a common subgenomic interval with the sample or test cfDNA fragment, and 4 are methylated and 6 are unmethylated (10 methylation sites in total). In such an example, the corresponding control cfDNA fragments are the control cfDNA fragments that align or overlap, in any part, within the same subgenomic interval (the dotted lines in FIG. 1B) as the sample or test cfDNA fragment (i.e., only part of the control cfDNA fragment aligns within the same subgenomic interval to correspond), the selected methylation sites in the sample or test cfDNA fragment (“M” or “U” in FIG. 1B) are the methylation sites that align or overlap with the same subgenomic interval (the dotted lines in FIG. 1B) as the corresponding control cfDNA fragments, and the selected methylation sites in the corresponding control cfDNA fragments are the methylation sites that align with the selected methylation sites in the sample or test cfDNA fragment.

The methylation fractions for selected methylation sites, in any of the instances described above, may be compared between sample or test cfDNA fragments and corresponding control cfDNA fragments to generate methylation fraction differences. In some aspects of the present invention, the methylation fraction difference is calculated as a subtracted difference between the methylation fraction for a sample cfDNA fragment or the methylation fraction for a test cfDNA fragment and the methylation fraction for corresponding control cfDNA fragments.

### Fragment Length Difference

In some aspects, a feature that may be calculated for cfDNA fragments is a fragment length difference between different types of cfDNA fragments. The fragment length difference compares the fragment length of a cfDNA fragment to the average fragment length of other cfDNA fragments. For example, a fragment length difference may be calculated as the difference between the fragment length of a sample cfDNA fragment and the average fragment length of corresponding control cfDNA fragments. Alternatively, a fragment length difference may be calculated as the difference between the fragment length of a test cfDNA fragment and the average fragment length of corresponding control cfDNA fragments. In some aspects, the average fragment length is calculated as a mean of cfDNA fragment lengths. In some aspects, the average fragment length is calculated as a median of cfDNA fragment lengths.

The calculation of a fragment length difference for a sample cfDNA fragment or a test cfDNA fragment compared to corresponding control cfDNA fragments involves a determination of fragment length. The length of a nucleic acid fragment may refer to the number of nucleotides within the entirety of the nucleic acid fragment. Alternatively, the length of a nucleic acid fragment may refer to the number of nucleotides within only the subset of the nucleic acid fragment that aligns or overlaps with a subgenomic interval.

In some aspects of the present invention, a sample cfDNA fragment may have one fragment length that describes the entire length of the sample cfDNA fragment that at least partially aligns with or overlaps a subgenomic interval. In some aspects, a sample cfDNA fragment may have one fragment length that describes the length of only the subset of the sample cfDNA fragment that aligns with or overlaps a subgenomic interval. In some aspects of the present invention, a test cfDNA fragment may have one fragment length that describes the entire length of the test cfDNA fragment that at least partially aligns with or overlaps a subgenomic interval. In some aspects, a test cfDNA fragment may have one fragment length that describes the length of only the subset of the test cfDNA fragment that aligns with or overlaps a subgenomic interval. In some aspects, corresponding control cfDNA fragments may have one average fragment length that describes the average of the entire lengths of all the corresponding control cfDNA fragments that at least partially align with or overlap a subgenomic interval. In some aspects, corresponding control cfDNA fragments may have one average fragment length that describes the average of the lengths of only the subset of corresponding control cfDNA fragments that align with or overlap a subgenomic interval.

For calculating a fragment length difference, the fragment length of a cfDNA fragment is compared to the average fragment length of other cfDNA fragments. For example, the fragment length of a sample cfDNA fragment can be compared to the average fragment length of corresponding control cfDNA fragments. In such an example, the sample cfDNA fragment can be any fragment that aligns or overlaps at least in part with a subgenomic interval, and the corresponding control cfDNA fragments can be all the control cfDNA fragments that align or overlap at least in part with the same subgenomic interval as the sample cfDNA fragment. Alternatively, the fragment length of a test cfDNA fragment can be compared to the average fragment length of corresponding control cfDNA fragments. In such an example, the test cfDNA fragment can be any fragment that aligns or overlaps at least in part with a subgenomic interval, and the corresponding control cfDNA fragments can be all the control cfDNA fragments that align or overlap at least in part with the same subgenomic interval as the test cfDNA fragment. In such an example, the test cfDNA fragment can be any fragment that aligns or overlaps at least in part with a subgenomic interval, and the corresponding control cfDNA fragments can be all the control cfDNA fragments that align or overlap at least in part with the same subgenomic interval as the test cfDNA fragment. In both cases, the corresponding control cfDNA fragments are the control cfDNA fragments that align with or overlap with a sample cfDNA fragment or a test cfDNA fragment by alignment to the same genomic position, or the control cfDNA fragments that align with the same subgenomic interval as a sample cfDNA fragment or a test cfDNA fragment.

With reference to FIG. 1C, for example, the fragment length of a sample or test cfDNA fragment may be determined and compared to the average fragment length of corresponding control cfDNA fragments, where the fragment length of the sample or test cfDNA fragment is the length of the entirety of the fragment, and the average fragment length of the corresponding control cfDNA fragments is the average of the lengths of the entirety of all the corresponding control cfDNA fragments. Alternatively, the fragment length of the sample or test cfDNA fragment may be the length of only the part of the fragment that aligns or overlaps with a subgenomic interval, and the average fragment length of the corresponding control cfDNA fragments may be the average of the lengths of only the parts of the corresponding control cfDNA fragments that align or overlap with the same subgenomic interval as the sample or test cfDNA fragment.

With reference to FIG. 1D, for example, the fragment length of a sample or test cfDNA fragment may be determined and compared to the average fragment length of corresponding control cfDNA fragments, where the fragment length of the sample or test cfDNA fragment is the length of the entirety of the fragment, and the average fragment length of the corresponding control cfDNA fragments is the average of the lengths of the entirety of all the corresponding control cfDNA fragments. Alternatively, the fragment length of the sample or test cfDNA fragment may be the length of only the part of the fragment that aligns or overlaps with a subgenomic interval, and the average fragment length of the corresponding control cfDNA fragments may be the average of the lengths of only the parts of the corresponding control cfDNA fragments that align or overlap with the same subgenomic interval as the sample or test cfDNA fragment. A cfDNA fragment may align or overlap with a subgenomic interval in its entirety (e.g., 102 in FIG. 1D), or it may align or overlap with a subgenomic interval only in part (e.g., 104 in FIG. 1D).

The fragment lengths, in any of the instances described above, may be compared between sample or test cfDNA fragments and corresponding control cfDNA fragments to generate fragment length differences. In some aspects of the present invention, the fragment length difference is a subtracted difference between the fragment length for a sample cfDNA fragment or the fragment length for a test cfDNA fragment and the average fragment length for corresponding control cfDNA fragments. In some aspects, the fragment length difference is a divided fraction between the fragment length for a sample cfDNA fragment or the fragment length for a test cfDNA fragment and the average fragment length for corresponding control cfDNA fragments.

Methods for Training a Filter Model for Filtering cfDNA Fragments

The disclosed methods for training a filter model for filtering sequencing data for cfDNA fragments can be executed using a computer-implemented method configured to analyze data for cfDNA fragments. The process considers features of the cfDNA fragments and uses them to train a model to be used for identifying whether cfDNA fragments are likely to be associated with disease. More specifically, the described process can consider values, methylation fraction differences, and fragment length differences, as described above, of the cfDNA fragments. These features can be used to set thresholds to be used in classifying whether cfDNA fragments are likely to be associated with disease. Being associated with disease might mean that a cfDNA fragment is derived from a diseased cell or diseased tissue, or that a cfDNA fragment is derived from a healthy cell or healthy tissue that has been impacted by disease. These features may be examined within different contexts, which might, for example, be genomic regions or subgenomic intervals that represent unique biological contexts.

The methods for training a filter model use sequencing data from a training sample obtained from a training subject having a disease. The sequencing data may comprise data for a plurality of cfDNA fragments from the training sample from the training subject, where the cfDNA fragments are referred to as sample cfDNA fragments or training sample cfDNA fragments, i.e., those fragments used for training a filter model. The described methods for training a filter model also use sequencing data from control samples obtained from control subjects labeled as not having disease. The sequencing data may comprise data for a plurality of cfDNA fragments from the control samples from the control subjects, where the cfDNA fragments are referred to as control cfDNA fragments or corresponding control cfDNA fragments meaning the fragments are used for comparison to sample cfDNA fragments in the context of training a filter model.

The methods herein comprise, for example, determining features of sample cfDNA fragments within subgenomic intervals and setting thresholds of a filter model based on those features that might be used to identify sample cfDNA fragments associated with disease. The features may include a value based on the methylation status of sample cfDNA fragments compared to control cfDNA fragments, a methylation fraction difference between control and sample cfDNA fragments, and a fragment length difference between control and sample cfDNA fragments. At least one, at least two, or all three of these features may be determined within any of the methods disclosed herein. The features may be measured in a wide range of biological contexts, which may also be referred to as genomic regions or subgenomic intervals.

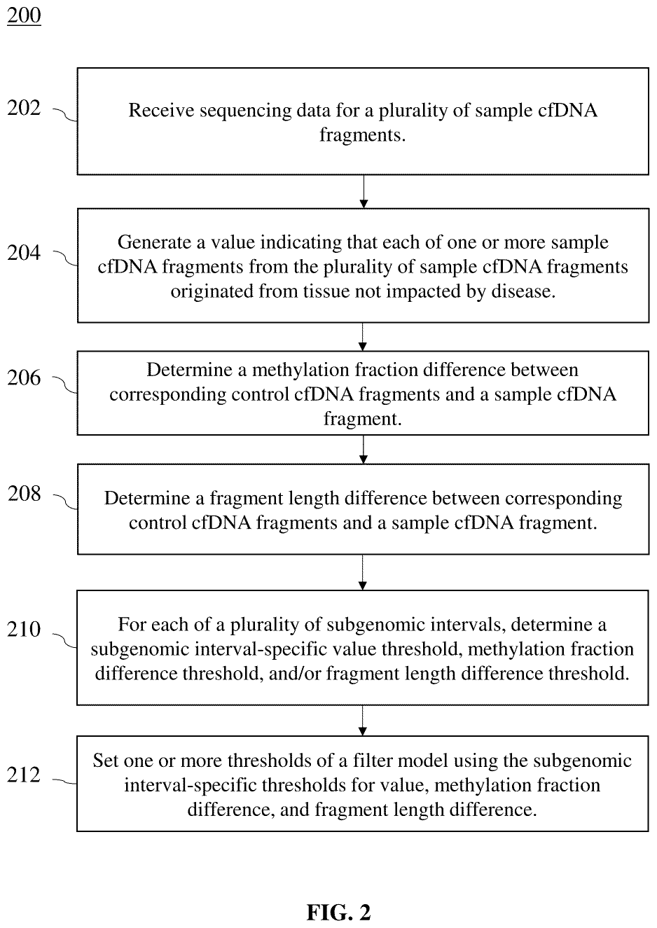

FIG. 2 provides a non-limiting exemplary schematic showing general process 200 for training a filter model for filtering sequencing data for cfDNA fragments. The method of training a filter model can include: receiving sequencing data for a plurality of sample cfDNA fragments (202); generating a value indicating that each of one or more sample cfDNA fragments from the plurality of sample cfDNA fragments originated from tissue not impacted by disease (204); determining a methylation fraction difference between corresponding control cfDNA fragments and a sample cfDNA fragment (206); determining a fragment length difference between corresponding control cfDNA fragments and a sample cfDNA fragment (208); for each of a plurality of subgenomic intervals, determining a subgenomic interval-specific value threshold, methylation fraction difference threshold, and fragment length difference threshold (210); and setting one or more thresholds of a filter model using the subgenomic interval-specific thresholds for value, methylation fraction difference, and fragment length difference (212).

### Training and Control Sample Selection

At 202 in FIG. 2, sequencing data is received for cfDNA fragments. Receiving sequencing data can include sequencing data from a training sample obtained from a training subject having a disease. The sequencing data may comprise data for a plurality of sample cfDNA fragments from the training sample from the training subject, where the sample cfDNA fragments are used for training a filter model. The training subject may have any type of disease. For example, the training sample can be obtained from a training subject with a hyperproliferative disease or a non-cancer disease. In some instances, the training subject has a hyperproliferative disease that is a cancer. In some instances, the training subject has a hematological cancer such as leukemia or lymphoma. In some instances, the training subject has a cardiovascular disease. In some instances, the training subject has a neurological disease. Receiving sequencing data can also include sequencing data from control samples obtained from control subjects labeled as not having disease. The sequencing data may comprise data for a plurality of control cfDNA fragments from the control samples from the control subjects, where the control cfDNA fragments are used for comparison to sample cfDNA fragments in the context of training a filter model. Sequencing data can be obtained from multiple control subjects all labeled as not having disease.

The sequencing data used for training a filter model can comprise information used to calculate features for sample cfDNA fragments compared to corresponding control cfDNA fragments. For example, the sequencing data may comprise information about methylation sites in a sample cfDNA fragment, or about methylation sites in a set of corresponding control fragments that correspond to the sample cfDNA fragment by aligning or overlapping with the same subgenomic interval as the sample cfDNA fragment. In a sample cfDNA fragment, information about methylation sites might include the number of methylated or unmethylated sites, the methylation status of the methylation sites, or the fraction of methylation sites that are methylated compared to the total number of methylation sites that are methylated or unmethylated. In a set of corresponding control cfDNA fragments, information about methylation sites might include the number of methylated or unmethylated sites in all the control cfDNA fragments, the methylation status of the methylation sites in all the control cfDNA fragments, or the fraction of methylation sites that are methylated compared to the total number of methylation sites that are methylated or unmethylated in all the control cfDNA fragments. This information may be used to calculate a value that compares methylation statuses between a sample cfDNA fragment and corresponding control cfDNA fragments, or a methylation fraction difference that compares methylation fractions between a sample cfDNA fragment and corresponding control cfDNA fragments. The sequencing data may also comprise information about fragment length of a sample cfDNA fragment, or about the average fragment length of a set of corresponding control fragments that correspond to the sample cfDNA fragment by aligning or overlapping with the same subgenomic interval as the sample cfDNA fragment. This information may be used to calculate a fragment length difference that compares fragment length of a sample cfDNA fragment to the average fragment length of corresponding control cfDNA fragments.

The features that may be calculated from sequencing data for a sample cfDNA fragment may include a methylation status for one or more first selected methylation sites in the sample cfDNA fragment, a methylation fraction for one or more second selected methylation sites in the sample cfDNA fragment, and a fragment length of the sample cfDNA fragment. The features that may be calculated from sequencing data for control cfDNA fragments may include a methylation status for one or more third selected methylation sites in the control cfDNA fragments, a methylation fraction for one or more fourth selected methylation sites in the control cfDNA fragments, and an average fragment length of the control cfDNA fragments.

The sample cfDNA fragments or the control cfDNA fragments may be sequenced to obtain the sequencing data to be used for training a filter model. Multiple sequencing approaches may be used to obtain the sequencing data for either type of fragment. For example, a massively parallel sequencing (MPS) technique, whole genome sequencing (WGS), whole exome sequencing, targeted sequencing, direct sequencing, or Sanger sequencing technique may be used to obtain the sequencing data for sample cfDNA fragments or the sequencing data for control cfDNA fragments. In some aspects, the sequencing comprises massively parallel sequencing. In some aspects, the sequencing comprises a next generation sequencing approach.

Extracting Features from the Training Sample cfDNA Fragments

With reference to FIG. 2, blocks 204, 206, and 208, training a filter model for filtering sequencing data for cfDNA fragments involves extracting features that describe sample cfDNA fragments and corresponding control cfDNA fragments. One or more features may be extracted for a sample cfDNA fragment compared to corresponding control cfDNA fragments using the methods as described above (value, methylation fraction difference, fragment length difference). In some instances, all three features are calculated for a given sample cfDNA fragment. In some instances, two of the three features are calculated for a given sample cfDNA fragment. The two features can be a value and a methylation fraction difference, or a value and a fragment length difference, or a methylation fraction difference and a fragment length difference. In some instances, one of the three features is calculated for a given sample cfDNA fragment. The one feature can be a value, a methylation fraction difference, or a fragment length difference. The features are used in the filter model to identify if cfDNA fragments are likely to have originated from tissue impacted by disease or not.

At 204 in FIG. 2, a value is generated. This value may be a statistical value. Each of one or more sample cfDNA fragments from the plurality of sample cfDNA fragments can have its own statistical value. Statistical values can be calculated as described above and with reference to FIGS. 1A-1B. The statistical value for a sample cfDNA fragment indicates the probability that the sample cfDNA fragment originated from tissue not impacted by disease. The statistical value may be calculated by comparing the methylation status for selected methylation sites in the sample cfDNA fragment and the methylation status for selected methylation sites in corresponding control cfDNA fragments. The selected methylation sites in a sample cfDNA fragment may be referred to as first selected methylation sites, and the selected methylation sites in corresponding control cfDNA fragments may be referred to as third selected methylation sites. In some aspects, the statistical value is a p-value. For example, the statistical value may be a p-value generated by a binomial test. Alternatively, the statistical value may be a p-value generated by another statistical test.

A statistical value can be calculated for each sample cfDNA fragment from a subset of a plurality of sample cfDNA fragments, or each sample cfDNA fragment from a plurality of sample cfDNA fragments, comparing the sample cfDNA fragment to corresponding control cfDNA fragments. Corresponding control cfDNA fragments are control cfDNA fragments that correspond to a given sample cfDNA fragment, within each subgenomic interval of a plurality of subgenomic intervals. As described above, the corresponding control cfDNA fragments to a sample cfDNA fragment are the control cfDNA fragments that align or overlap with the sample cfDNA fragment in any part. The corresponding control cfDNA fragments may also align with the same subgenomic interval as the sample cfDNA fragment. The corresponding control cfDNA fragments may also overlap with the same subgenomic interval as the sample cfDNA fragment.

For the purposes of calculating a statistical value, as described above, methylation sites in corresponding control cfDNA fragments are compared to methylation sites in a sample cfDNA fragment. In both types of cfDNA fragments, only a subset of methylation sites may be compared to calculate a statistical value. The subset of methylation sites may be referred to as selected methylation sites. Methylation sites may be selected in certain combinations in both a sample cfDNA fragment and corresponding control cfDNA fragments. For example, in some instances, the selected methylation sites are all the methylation sites in the entire length of the sample cfDNA fragment, and all the methylation sites in the corresponding control cfDNA fragments. In some instances, the selected methylation sites all the methylation sites in the sample cfDNA fragment, and only the methylation sites in corresponding control cfDNA fragments that positionally overlap with the selected methylation sites in the sample cfDNA fragment and that align within a common subgenomic interval with the sample cfDNA fragment. In some instances, the selected methylation sites are all the methylation sites in the corresponding control cfDNA fragments, and only the methylation sites in a sample cfDNA fragment that positionally overlap with the selected methylation sites in the corresponding control cfDNA fragments and that align within a common subgenomic interval with the corresponding control cfDNA fragments. In some instances, the selected methylation sites are only the methylation sites in a sample cfDNA fragment that positionally overlap with the selected methylation sites in the corresponding control cfDNA fragments and that align within a common subgenomic interval with the corresponding control cfDNA fragments, and only the methylation sites in corresponding control cfDNA fragments that positionally overlap with the selected methylation sites in the sample cfDNA fragment and that align within a common subgenomic interval with the sample cfDNA fragment.

At 206 in FIG. 2, a methylation fraction difference is determined. Each of one or more sample cfDNA fragments from the plurality of sample cfDNA fragments can have its own methylation fraction difference. Methylation fraction differences may be calculated as described above and with reference to FIGS. 1A-1B. The methylation fraction difference for a sample cfDNA fragment indicates the difference between a methylation fraction for a sample cfDNA fragment and a methylation fraction for corresponding control fragments. Methylation fraction describes the proportion of methylation sites that are methylated, compared to the total number of methylated and unmethylated methylation sites. Methylation fraction can be calculated for sample cfDNA fragments and for control cfDNA fragments. For sample and control cfDNA fragments, the methylation fraction may describe a proportion relating to all the methylated or unmethylated methylation sites within the entirety of a cfDNA fragment, or a proportion relating to only selected methylated or unmethylated methylation sites within a portion of a cfDNA fragment. For calculating a methylation fraction, the selected methylation sites in a sample cfDNA fragment may be referred to as second selected methylation sites, and the selected methylation sites in a control cfDNA fragment may be referred to as fourth selected methylation sites. In some aspects, the methylation fraction difference is a subtracted difference between the methylation fraction for selected methylation sites in a sample cfDNA fragment and the methylation fraction for selected methylation sites in corresponding control cfDNA fragments. In some aspects, the second selected methylation sites in sample cfDNA fragments used for calculating a methylation fraction difference are the same methylation sites as the first selected methylation sites in sample cfDNA fragments used for calculating a value or a statistical value based on methylation statuses. In some aspects, the second selected methylation sites in sample cfDNA fragments used for calculating a methylation fraction difference are different methylation sites from the first selected methylation sites in sample cfDNA fragments used for calculating a value or a statistical value based on methylation statuses. In some aspects, the fourth selected methylation sites in control cfDNA fragments used for calculating a methylation fraction difference are the same methylation sites as the third selected methylation sites for calculating a value or a statistical value based on methylation statuses. In some aspects, the fourth selected methylation sites in control cfDNA fragments used for calculating a methylation fraction difference are different methylation sites from the third selected methylation sites in control cfDNA fragments used for calculating a value or a statistical value based on methylation statuses.

A methylation fraction difference can be determined for each sample cfDNA fragment from a subset of a plurality of sample cfDNA fragments, or each sample cfDNA fragment from a plurality of cfDNA fragments, comparing the sample cfDNA fragment to corresponding control cfDNA fragments. Corresponding control cfDNA fragments are control cfDNA fragments that correspond to a given sample cfDNA fragment, within each subgenomic interval of a plurality of subgenomic intervals. As described above, the corresponding control cfDNA fragments to a sample cfDNA fragment are the control cfDNA fragments that align or overlap with the sample cfDNA fragment in any part. The corresponding control cfDNA fragments may also align with the same subgenomic interval as the sample cfDNA fragment. The corresponding control cfDNA fragments may also overlap with the same subgenomic interval as the sample cfDNA fragment.

For the purposes of calculating a methylation fraction difference, as described above, a methylation fraction of corresponding control cfDNA fragments is compared to a methylation fraction in a sample cfDNA fragment. In both types of fragments, only a subset of methylation sites may be compared to determine a methylation fraction that describes the proportion of methylated methylation sites. The subset of methylation sites may be referred to as selected methylation sites. Methylation sites may be selected in certain combinations in both a sample cfDNA fragment and corresponding control cfDNA fragments. For example, in some instances, the selected methylation sites are all the methylation sites in the entire length of the sample cfDNA fragment, and all the methylation sites in the corresponding control cfDNA fragments. In some instances, the selected methylation sites all the methylation sites in the sample cfDNA fragment, and only the methylation sites in corresponding control cfDNA fragments that positionally overlap with the selected methylation sites in the sample cfDNA fragment and that align within a common subgenomic interval with the sample cfDNA fragment. In some instances, the selected methylation sites are all the methylation sites in the corresponding control cfDNA fragments, and only the methylation sites in a sample cfDNA fragment that positionally overlap with the selected methylation sites in the corresponding control cfDNA fragments and that align within a common subgenomic interval with the corresponding control cfDNA fragments. In some instances, the selected methylation sites are only the methylation sites in a sample cfDNA fragment that positionally overlap with the selected methylation sites in the corresponding control cfDNA fragments and that align within a common subgenomic interval with the corresponding control cfDNA fragments, and only the methylation sites in corresponding control cfDNA fragments that positionally overlap with the selected methylation sites in the sample cfDNA fragment and that align within a common subgenomic interval with the sample cfDNA fragment.

At 208 in FIG. 2, a fragment length difference is determined. Each of one or more sample cfDNA fragments from the plurality of sample cfDNA fragments can have its own fragment length difference. Fragment length differences may be calculated as described above and with reference to FIGS. 1C-1D. The fragment length difference for a sample cfDNA fragment indicates the difference between fragment length for the sample cfDNA fragment and the average fragment length of corresponding control cfDNA fragments. In some aspects, the fragment length difference is a subtracted difference between the fragment length of a sample cfDNA fragment and the average fragment length of corresponding control cfDNA fragments. In some aspects, the fragment length difference is a divided fraction between the fragment length of a sample cfDNA fragment and the average fragment length corresponding control cfDNA fragments.

A fragment length difference can be determined for each sample cfDNA fragment from a subset of a plurality of sample cfDNA fragments, or each sample cfDNA fragment from a plurality of cfDNA fragments, comparing the sample cfDNA fragment to corresponding control cfDNA fragments. Corresponding control cfDNA fragments are control cfDNA fragments that correspond to a given sample cfDNA fragment, within each subgenomic interval of a plurality of subgenomic intervals. As described above, the corresponding control cfDNA fragments to a sample cfDNA fragment are the control cfDNA fragments that align or overlap with the sample cfDNA fragment in any part. The corresponding control cfDNA fragments may also align with the same subgenomic interval as the sample cfDNA fragment. The corresponding control cfDNA fragments may also overlap with the same subgenomic interval as the sample cfDNA fragment.

For the purposes of calculating a fragment length difference, as described above, the average fragment length of corresponding control cfDNA fragments is compared to the fragment length of a sample cfDNA fragment. For both sample and control cfDNA fragments, fragment length may describe the length of the entirety of the fragment or the length of only part of the fragment that aligns or overlaps with a subgenomic interval. For example, the fragment length of the sample cfDNA fragment may be the length of the entirety of the fragment, and the average fragment length of the corresponding control cfDNA fragments may be the average of the lengths of the entirety of all the corresponding control cfDNA fragments. Alternatively, the fragment length of the sample cfDNA fragment may be the length of only the part of the fragment that aligns or overlaps with a subgenomic interval, and the average fragment length of the corresponding control cfDNA fragments may be the average of the lengths of only the parts of the corresponding control cfDNA fragments that align or overlap with the same subgenomic interval as the sample cfDNA fragment.

### Determining and Setting Thresholds

At 210 in FIG. 2, subgenomic interval-specific thresholds are determined. For each of a plurality of subgenomic intervals, a subgenomic interval-specific threshold can be determined for each of the features extracted for the sample cfDNA fragments. For each subgenomic interval, a value threshold, methylation fraction difference threshold, and/or fragment length difference threshold are determined. These thresholds are determined using the features (i.e., value, methylation fraction difference, fragment length difference) extracted as described above and with reference to FIGS. 1A-1D, for sample cfDNA fragments that align or overlap with each subgenomic interval. In some cases, the methods disclosed herein are performed in a context limited to only cfDNA fragments within a specific range of lengths. For examples, the methods herein may consider only cfDNA fragments between about 65 base pairs in length to about 140 base pairs in length, between about 140 base pairs in length to about 200 base pairs in length, or between about 280 base pairs in length to about 600 base pairs in length.

A threshold may be a numerical value that represents a minimum cutoff. Determining the subgenomic interval-specific thresholds may include determining a plurality of receiver operating characteristic (ROC) curves using a plurality of different subgenomic interval-specific value thresholds, a plurality of different subgenomic interval-specific methylation fraction difference thresholds, and a plurality of different subgenomic interval-specific fragment length difference thresholds and selecting the subgenomic interval-specific value threshold, the subgenomic interval-specific methylation fraction difference threshold, and/or the subgenomic interval-specific fragment length difference threshold providing the highest area under the curve (AUC) among the plurality of ROC curves.

In some aspects, the subgenomic interval-specific thresholds are determined using a grid search that examines different sets of thresholds for how well each set of thresholds (i.e., the value threshold, the methylation fraction difference threshold, and/or the fragment length difference threshold in a subgenomic interval) can differentiate between cfDNA fragments associated with disease and cfDNA fragments not associated with disease. The grid search can be done for each subgenomic interval such that each interval has a set of optimal thresholds. By considering values at every point in the grid, this approach can identify the optimal set of subgenomic interval-specific thresholds for each subgenomic interval, identifying a set of a value threshold, a methylation fraction difference threshold, and/or a fragment length difference threshold that are the best at separating between cfDNA fragments associated with disease and cfDNA fragments not associated with disease. The grid search approach can be used to identify a unique set of thresholds for each subgenomic interval of a plurality of subgenomic intervals.

At 212 in FIG. 2, one or more thresholds of a filter model are set. The thresholds can be set using the subgenomic interval-specific thresholds for value, methylation fraction difference, and/or fragment length difference, having been determined from the grid search as described above. That is, after picking the highest AUC, the corresponding value, methylation fraction difference, and/or fragment length difference can be set as thresholds within each subgenomic interval. The resulting thresholds of the filter model can be applied to other downstream classifier models used for data classification.

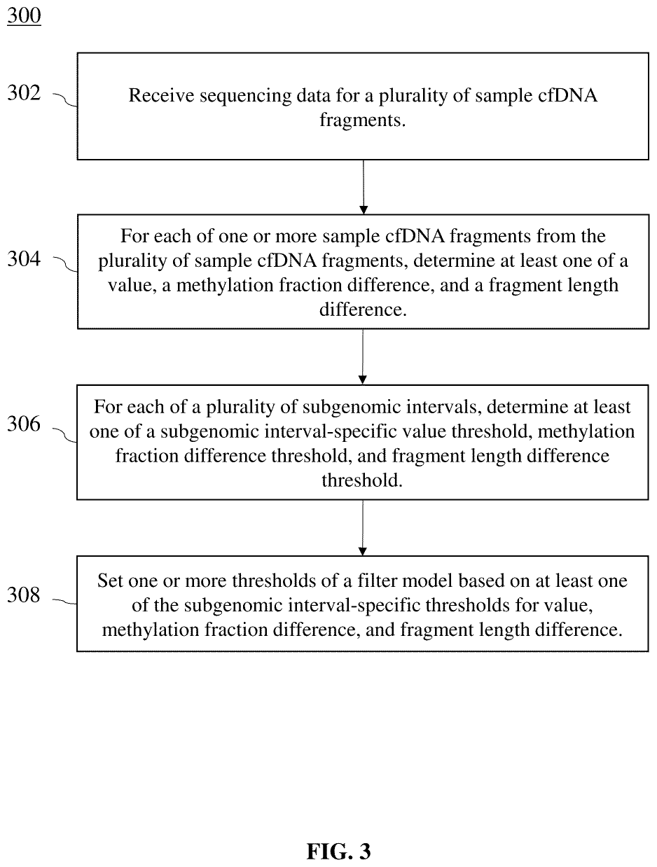

FIG. 3 provides another non-limiting exemplary schematic showing general process 300 for training a filter model for filtering sequencing data for cfDNA fragments. The method of training a filter model can include: receiving sequencing data for a plurality of sample cfDNA fragments (302); for each of one or more sample cfDNA fragments from the plurality of sample cfDNA fragments, determining at least one of a value, a methylation fraction difference, and a fragment length difference (304); for each of a plurality of subgenomic intervals, determining at least one of a subgenomic interval-specific value threshold, methylation fraction difference threshold, and fragment length difference threshold (306); and setting one or more thresholds of a filter model based on at least one of the subgenomic interval-specific thresholds for value, methylation fraction difference, and fragment length difference (308).

At 302 in FIG. 3, sequencing data is received for cfDNA fragments, as described for block 202 in FIG. 2, including data for sample cfDNA fragments and control cfDNA fragments. At 304 in FIG. 3, at least one feature is extracted from the sample cfDNA fragments, following the methods described for blocks 204, 206, and/or 208 in FIG. 2. For each sample cfDNA fragment from a plurality of sample cfDNA fragments, at least one of a value, methylation fraction difference, fragment length difference can be determined, as described above. The feature can then be used in the filter model to identify if cfDNA fragments are likely to have originated from tissue impacted by disease or not. In some aspects, at least two of a value, a methylation fraction difference, and a fragment length difference are determined. For example, a value and a methylation fraction difference may be determined; a value and a fragment length difference may be determined; or a methylation fraction difference and a fragment length difference may be determined. Alternatively, only one of a value, a methylation fraction difference, or a fragment length difference may be determined; or all three of a value, a methylation fraction difference, or a fragment length difference may be determined. The value may be determined with reference to FIGS. 1A-1B and block 204 in FIG. 2; the methylation fraction difference may be determined with reference to FIGS. 1A-1B and block 206 in FIG. 2; and the fragment length difference may be determined with reference to FIGS. 1C-1D and block 208 in FIG. 2.

At 306 in FIG. 3, subgenomic interval-specific thresholds are determined. For each of a plurality of subgenomic intervals, at least one of a subgenomic interval-specific value threshold, methylation fraction difference threshold, and fragment length difference threshold is determined. In some aspects, at least two of a subgenomic interval-specific value threshold, methylation fraction difference threshold, and fragment length difference threshold may be determined. For example, a subgenomic interval-specific value threshold and a subgenomic interval-specific methylation fraction difference threshold may be determined; a subgenomic interval-specific value threshold and a subgenomic interval-specific fragment length difference threshold may be determined; or a subgenomic interval-specific methylation fraction difference threshold and a subgenomic interval-specific fragment length difference threshold may be determined. Alternatively, only one of a subgenomic interval-specific value threshold, subgenomic interval-specific methylation fraction difference threshold, or subgenomic interval-specific fragment length difference threshold may be determined; or all three of a subgenomic interval-specific value threshold, subgenomic interval-specific methylation fraction difference threshold, and subgenomic interval-specific fragment length difference threshold may be determined.

A threshold may be a numerical value that represents a minimum cutoff. Determining the subgenomic interval-specific thresholds may include determining a plurality of receiver operating characteristic (ROC) curves using a plurality of different subgenomic interval-specific value thresholds, a plurality of different subgenomic interval-specific methylation fraction difference thresholds, and a plurality of different subgenomic interval-specific fragment length difference thresholds and selecting the subgenomic interval-specific value threshold, the subgenomic interval-specific methylation fraction difference threshold, and the subgenomic interval-specific fragment length difference threshold providing the highest area under the curve (AUC) among the plurality of ROC curves.

At 308 in FIG. 3, one or more thresholds of a filter model are set. The thresholds can be set using at least one of the subgenomic interval-specific thresholds for value, methylation fraction difference, and fragment length difference. In some aspects, at least two of a subgenomic interval-specific value threshold, subgenomic interval-specific methylation fraction difference threshold, or subgenomic interval-specific fragment length difference threshold may be used to set the thresholds of a filter model. For example, a subgenomic interval-specific value threshold and a subgenomic interval-specific methylation fraction difference threshold may be used; a subgenomic interval-specific value threshold and a subgenomic interval-specific fragment length difference threshold may be used; or a subgenomic interval-specific methylation fraction difference threshold and a subgenomic interval-specific fragment length difference threshold may be used. Alternatively, only one of a subgenomic interval-specific value threshold, subgenomic interval-specific methylation fraction difference threshold, or subgenomic interval-specific fragment length difference threshold may be used; or all three of a subgenomic interval-specific value threshold, subgenomic interval-specific methylation fraction difference threshold, and subgenomic interval-specific fragment length difference threshold may be used.

The thresholds can be set within each subgenomic interval. The filter model can be any classifier model used for classification of data, which may include neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can include the use of dimensionality reduction techniques (e.g., principal component analysis, matrix factorization techniques, and/or autoencoders).

### Using the Filter Model and Types Of Filter Models

The subgenomic interval-specific thresholds are used to set filters of a filter model, which is used to filter sequencing data for cfDNA fragments. Sequencing data for cfDNA fragments can be filtered depending on whether features of the cfDNA fragments, determined as described above, pass through subgenomic interval-specific thresholds or not. For example, a subgenomic interval-specific threshold may be a numerical value that is a minimum cutoff, and a cfDNA fragment that passes through the threshold may be one with a feature that is higher than the cutoff value of the threshold. If a cfDNA fragment passes through a threshold, it may be described as having been filtered.

Filtering using the cutoff values of the thresholds can be done in each of a plurality of subgenomic intervals and can be used to identify an association with disease or not. That is, filtering can mean identifying and labeling that certain cfDNA fragments originated from tissue impacted by disease, and that other cfDNA fragments originated from tissue not impacted by disease. Filtering can also include identifying a specific type of disease, i.e., that certain cfDNA fragments originated from tissue impacted by one type of disease and other cfDNA fragments originated from tissue impacted by another type of disease. Filtering can also include identifying a specific stage of disease, for example, that certain cfDNA fragments originated from tissue impacted by early-stage disease and other cfDNA fragments originated from tissue impacted by late-stage disease. In any of the above instances, the disease may be a hyperproliferative or non-proliferative disease, a type of cancer, a type of hematological cancer, a neurological disease, or a cardiovascular disease.

The methods of training a filter model, with reference to FIG. 2 or FIG. 3, can also be used to train multiple types of filter models. Different types of filter models can be trained for different types of disease. The different types of diseases can include different types of hyperproliferative or non-proliferative diseases, cancers, cardiovascular diseases, or neurological diseases. Different types of filter models can also be trained for different disease states or stages. For example, filter models may be trained for early-stage or late-stage diseases. In any of these instances, different types of training samples from training subjects are used to train different types of filter models. As described above, the training subject from which the sequencing data is received can have any type of disease. For example, the training sample can be obtained from a training subject with a hyperproliferative disease that is a cancer, and the sequencing data that is received can be used to train a filter model specific to the cancer of the training subject. A training sample can be obtained from a training subject with a non-proliferative disease, and the sequencing data that is received can be used to train a filter model specific to the non-proliferative disease of the training subject. The non-proliferative disease may be a cardiovascular disease or a neurological disease.

In instances where the sample is derived from a training subject with a cancer and the filter model is trained for that type of cancer, the cancer may be a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some instances, the cancer may comprise acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

Process 200 or 300 can be performed, for example, using one or more electronic devices implementing a software platform. In some examples, process 200 or 300 is performed using a client-server system, and the blocks of process 200 or 300 are divided up in any manner between the server and a client device. In other examples, the blocks of process 200 or 300 are divided up between the server and multiple client devices. Thus, while portions of process 200 or 300 are described herein as being performed by particular devices of a client-server system, it will be appreciated that process 200 or 300 is not so limited. In other examples, process 200 or 300 is performed using only a client device or only multiple client devices. In process 200 or 300, some blocks are, optionally, combined, the order of some blocks is, optionally, changed, and some blocks are, optionally, omitted. In some examples, additional steps may be performed in combination with the process 200 or 300. Accordingly, the operations as illustrated (and described in greater detail below) are exemplary by nature and, as such, should not be viewed as limiting.

Methods for Labeling Test cfDNA Fragments from a Test Subject Using a Filter Model

Test cfDNA fragments from a test subject may be labeled using a trained filter model, for example a trained filter model described above in reference to FIG. 2 and FIG. 3. The cfDNA fragments may be labeled using a computer-implemented process. The labels may indicate that a given cfDNA fragment originates from tissue impacted by the disease (i.e., the disease for which the trained filter model is trained) or not. Being associated with disease might mean that a cfDNA fragment is derived from a diseased cell or diseased tissue, or that a cfDNA fragment is derived from a healthy cell or healthy tissue that has been impacted by disease. These features may be examined within different contexts, which might, for example, be genomic regions or subgenomic intervals that represent unique biological contexts.

The methods for labeling cfDNA fragments described herein use sequencing data from a test sample obtained from a test subject. The test subject can have an unknown disease state, meaning that the test subject may have no disease or a specific type or stage of disease. The sequencing data may comprise data for a plurality of cfDNA fragments from the test sample from the test subject, where the cfDNA fragments are referred to as test cfDNA fragments meaning the fragments are to be used as input for a trained filter model as described in FIG. 2 or FIG. 3, and ultimately labeled as associated with disease or not. The described methods for labeling cfDNA fragments also use sequencing data from control samples obtained from control subjects labeled as not having disease. The sequencing data may comprise data for a plurality of cfDNA fragments from the control samples from the control subjects, where the cfDNA fragments are referred to as control cfDNA fragments or corresponding control cfDNA fragments meaning the fragments are used for comparison to test cfDNA fragments in the context of calculating features for the test cfDNA fragments and then labeling the test cfDNA fragments as associated with disease or not using a trained filter model.

The methods herein comprise, for example, determining features of test cfDNA fragments within subgenomic intervals and using those features to ultimately label test cfDNA fragments as being associated with disease or not. The features may include a value based on the methylation status of test cfDNA fragments compared to control cfDNA fragments, a methylation fraction difference between control and test cfDNA fragments, and a fragment length difference between control and test cfDNA fragments. At least one, at least two, or all three of these features may be determined within any of the methods disclosed herein. The features may be measured in a wide range of biological contexts, which may also be referred to as genomic regions or subgenomic intervals.

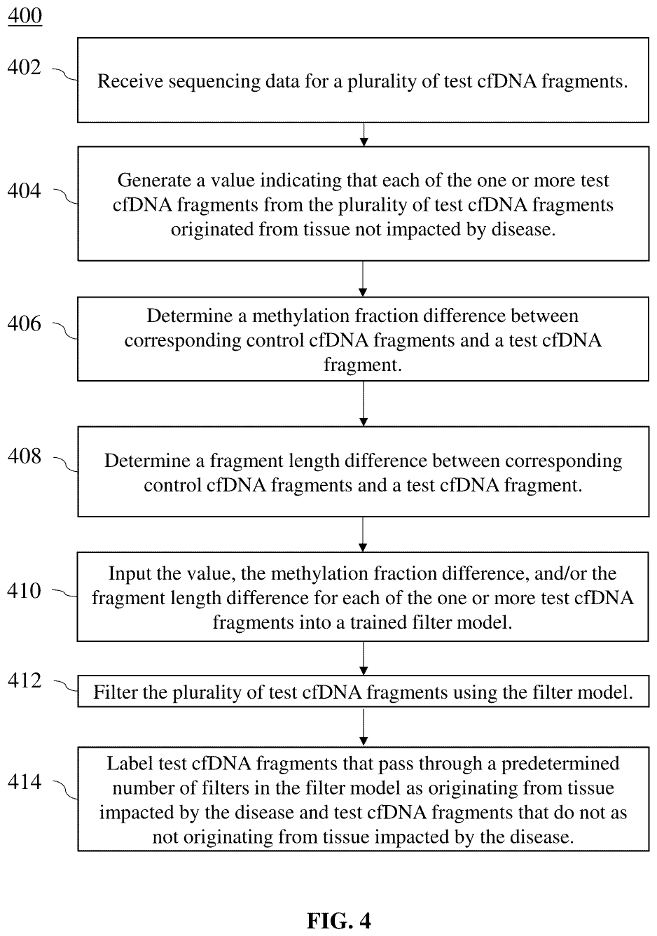

FIG. 4 provides a non-limiting exemplary schematic showing general process 400 for labeling test cfDNA fragments from a test subject. The method of labeling test cfDNA fragments can include: receiving sequencing data for a plurality of test cfDNA fragments (402); generating a value indicating that each of the one or more test cfDNA fragments from the plurality of test cfDNA fragments originated from tissue not impacted by disease (404); determining a methylation fraction difference between corresponding control cfDNA fragments and a test cfDNA fragment (406); determining a fragment length difference between corresponding control cfDNA fragments and a test cfDNA fragment (408); inputting the value, the methylation fraction difference, and the fragment length difference for each of the one or more test cfDNA fragments into a trained filter model (410); filtering the plurality of test cfDNA fragments using the filter model (412); and labeling test cfDNA fragments that pass through a predetermined number of filters in the filter model as originating from tissue impacted by the disease and test cfDNA fragments that do not as not originating from tissue impacted by the disease (414).

### Test and Control Sample Selection

At 402 in FIG. 4, sequencing data is received for cfDNA fragments. Receiving sequencing data can include sequencing data from a test sample obtained from a test subject with an unknown disease state. The sequencing data may comprise data for a plurality of test cfDNA fragments from the test sample from the test subject, where the test cfDNA fragments will be input into a trained filter model and then labeled as associated with disease or not. The test subject having an unknown disease state may be determined to have a disease, be suspected of having a disease, or may not have a disease. Should the test subject have a disease, the disease may be a hyperproliferative disease or a non-cancer disease. In some instances, the test subject has a hyperproliferative disease that is a cancer. In some instances, the test subject has a hematological cancer such as leukemia or lymphoma. In some instances, the test subject has a cardiovascular disease. In some instances, the test subject has a neurological disease. Receiving sequencing data can also include sequencing data from control samples obtained from control subjects labeled as not having disease. The sequencing data may comprise data for a plurality of control cfDNA fragments from the control samples from the control subjects, where the control cfDNA fragments are used for comparison to test cfDNA fragments in the context of calculating features for the test cfDNA fragments. Sequencing data can be obtained from multiple control subjects all labeled as not having disease.

The sequencing data can comprise information used to calculate features for test cfDNA fragments compared to corresponding control cfDNA fragments. For example, the sequencing data may comprise information about methylation sites in a test cfDNA fragment, or about methylation sites in a set of corresponding control fragments that correspond to the test cfDNA fragment by aligning or overlapping with the same subgenomic interval as the test cfDNA fragment. In a test cfDNA fragment, information about methylation sites might include the number of methylated or unmethylated sites, the methylation status of the methylation sites, or the fraction of methylation sites that are methylated compared to the total number of methylation sites that are methylated or unmethylated. In a set of corresponding control cfDNA fragments, information about methylation sites might include the number of methylated or unmethylated sites in all the control cfDNA fragments, the methylation status of the methylation sites in all the control cfDNA fragments, or the fraction of methylation sites that are methylated compared to the total number of methylation sites that are methylated or unmethylated in all the control cfDNA fragments. This information may be used to calculate a value that compares methylation statuses between a test cfDNA fragment and corresponding control cfDNA fragments, or a methylation fraction difference that compares methylation fractions between a test cfDNA fragment and corresponding control cfDNA fragments. The sequencing data may also comprise information about fragment length of a test cfDNA fragment, or about the average fragment length of a set of corresponding control fragments that correspond to the test cfDNA fragment by aligning or overlapping with the same subgenomic interval as the test cfDNA fragment. This information may be used to calculate a fragment length difference that compares fragment length of a test cfDNA fragment to the average fragment length of corresponding control cfDNA fragments.

The features that may be calculated from sequencing data for a test cfDNA fragment may include a methylation status for one or more first selected methylation sites in the test cfDNA fragment, a methylation fraction for one or more second selected methylation sites in the test cfDNA fragment, and a fragment length of the test cfDNA fragment. The features that may be calculated from sequencing data for control cfDNA fragments may include a methylation status for one or more third selected methylation sites in the control cfDNA fragments, a methylation fraction for one or more fourth selected methylation sites in the control cfDNA fragments, and an average fragment length of the control cfDNA fragments.

The test cfDNA fragments or the control cfDNA fragments may be sequenced to obtain the sequencing data to be used in the methods described herein. Multiple sequencing approaches may be used to obtain the sequencing data for either type of fragment. For example, a massively parallel sequencing (MPS) technique, whole genome sequencing (WGS), whole exome sequencing, targeted sequencing, direct sequencing, or Sanger sequencing technique may be used to obtained the sequencing data for test cfDNA fragments or the sequencing data for control cfDNA fragments. In some aspects, the sequencing comprises massively parallel sequencing. In some aspects, the sequencing comprises a next generation sequencing approach.

Extracting Features from the Test cfDNA Fragments

With reference to FIG. 4, blocks 404, 406, and 408, features are extracted that describe test cfDNA fragments and corresponding control cfDNA fragments. One or more features may be extracted for a test cfDNA fragment compared to corresponding control cfDNA fragments using the methods as described above (value, methylation fraction difference, fragment length difference). In some instances, all three features are calculated for a given test cfDNA fragment. In some instances, two of the three features are calculated for a given test cfDNA fragment. The two features can be a value and a methylation fraction difference, or a value and a fragment length difference, or a methylation fraction difference and a fragment length difference. In some instances, one of the three features is calculated for a given test cfDNA fragment. The one feature can be a value, a methylation fraction difference, or a fragment length difference. The features are used in the trained filter model to label if test cfDNA fragments originated from tissue impacted by disease or not.

At 404 in FIG. 4, a value is generated. This value can be a statistical value. Each of one or more test cfDNA fragments from the plurality of test cfDNA fragments can have its own statistical value. Statistical values can be calculated as described above and with reference to FIGS. 1A-1B. The statistical value for a test cfDNA fragment indicates the probability that the test cfDNA fragment originated from tissue not impacted by disease. The statistical value may be calculated by comparing the methylation status for selected methylation sites in the test cfDNA fragment and the methylation status for selected methylation sites in corresponding control cfDNA fragments. The selected methylation sites in a test cfDNA fragment may be referred to as first selected methylation sites, and the selected methylation sites in corresponding control cfDNA fragments may be referred to as third selected methylation sites. In some aspects, the statistical value is a p-value. For example, the statistical value may be a p-value generated by a binomial test. Alternatively, the statistical value may be a p-value generated by another statistical test.

A statistical value can be calculated for each test cfDNA fragment from a subset of a plurality of test cfDNA fragments, or each test cfDNA fragment from a plurality of test cfDNA fragments, comparing the test cfDNA fragment to corresponding control cfDNA fragments. Corresponding control cfDNA fragments are control cfDNA fragments that correspond to a given test cfDNA fragment, within each subgenomic interval of a plurality of subgenomic intervals. As described above, the corresponding control cfDNA fragments to a test cfDNA fragment are the control cfDNA fragments that align or overlap with the test cfDNA fragment in any part. The corresponding control cfDNA fragments may also align with the same subgenomic interval as the test cfDNA fragment. The corresponding control cfDNA fragments may also overlap with the same subgenomic interval as the test cfDNA fragment.

For the purposes of calculating a statistical value, as described above, methylation sites in corresponding control cfDNA fragments are compared to methylation sites in a test cfDNA fragment. In both types of cfDNA fragments, only a subset of methylation sites may be compared to calculate a statistical value. The subset of methylation sites may be referred to as selected methylation sites. Methylation sites may be selected in certain combinations in both a test cfDNA fragment and corresponding control cfDNA fragments. For example, in some instances, the selected methylation sites are all the methylation sites in the entire length of the test cfDNA fragment, and all the methylation sites in the corresponding control cfDNA fragments. In some instances, the selected methylation sites all the methylation sites in the test cfDNA fragment, and only the methylation sites in corresponding control cfDNA fragments that positionally overlap with the selected methylation sites in the test cfDNA fragment and that align within a common subgenomic interval with the test cfDNA fragment. In some instances, the selected methylation sites are all the methylation sites in the corresponding control cfDNA fragments, and only the methylation sites in a test cfDNA fragment that positionally overlap with the selected methylation sites in the corresponding control cfDNA fragments and that align within a common subgenomic interval with the corresponding control cfDNA fragments. In some instances, the selected methylation sites are only the methylation sites in a test cfDNA fragment that positionally overlap with the selected methylation sites in the corresponding control cfDNA fragments and that align within a common subgenomic interval with the corresponding control cfDNA fragments, and only the methylation sites in corresponding control cfDNA fragments that positionally overlap with the selected methylation sites in the test cfDNA fragment and that align within a common subgenomic interval with the test cfDNA fragment.

At 406 in FIG. 4, a methylation fraction difference is determined. Each of one or more test cfDNA fragments from the plurality of test cfDNA fragments can have its own methylation fraction difference. Methylation fraction differences may be calculated as described above and with reference to FIGS. 1A-1B. The methylation fraction difference for a test cfDNA fragment indicates the difference between a methylation fraction for a test cfDNA fragment and a methylation fraction for corresponding control fragments. Methylation fraction describes the proportion of methylation sites that are methylated, compared to the total number of methylated and unmethylated methylation sites. Methylation fraction can be calculated for test cfDNA fragments and for control cfDNA fragments. For test and control cfDNA fragments, the methylation fraction may describe a proportion relating to all the methylated or unmethylated methylation sites within the entirety of a cfDNA fragment, or a proportion relating to only selected methylated or unmethylated methylation sites within a portion of a cfDNA fragment. For calculating a methylation fraction, the selected methylation sites in a test cfDNA fragment may be referred to as second selected methylation sites, and the selected methylation sites in a control cfDNA fragment may be referred to as fourth selected methylation sites. In some aspects, the methylation fraction difference is a subtracted difference between the methylation fraction for selected methylation sites in a test cfDNA fragment and the methylation fraction for selected methylation sites in corresponding control cfDNA fragments. In some aspects, the second selected methylation sites in test cfDNA fragments used for calculating a methylation fraction difference are the same methylation sites as the first selected methylation sites in test cfDNA fragments used for calculating a value or a statistical value based on methylation statuses. In some aspects, the second selected methylation sites in test cfDNA fragments used for calculating a methylation fraction difference are different methylation sites from the first selected methylation sites in test cfDNA fragments used for calculating a value or a statistical value based on methylation statuses. In some aspects, the fourth selected methylation sites in control cfDNA fragments used for calculating a methylation fraction difference are the same methylation sites as the third selected methylation sites for calculating a value or a statistical value based on methylation statuses. In some aspects, the fourth selected methylation sites in control cfDNA fragments used for calculating a methylation fraction difference are different methylation sites from the third selected methylation sites in control cfDNA fragments used for calculating a value or a statistical value based on methylation statuses.

A methylation fraction difference can be determined for each test cfDNA fragment from a subset of a plurality of test cfDNA fragments, or each test cfDNA fragment from a plurality of cfDNA fragments, comparing the test cfDNA fragment to corresponding control cfDNA fragments. Corresponding control cfDNA fragments are control cfDNA fragments that correspond to a given test cfDNA fragment, within each subgenomic interval of a plurality of subgenomic intervals. As described above, the corresponding control cfDNA fragments to a test cfDNA fragment are the control cfDNA fragments that align or overlap with the test cfDNA fragment in any part. The corresponding control cfDNA fragments may also align with the same subgenomic interval as the test cfDNA fragment. The corresponding control cfDNA fragments may also overlap with the same subgenomic interval as the test cfDNA fragment.

For the purposes of calculating a methylation fraction difference, as described above, a methylation fraction of corresponding control cfDNA fragments is compared to a methylation fraction in a test cfDNA fragment. In both types of fragments, only a subset of methylation sites may be compared to determine a methylation fraction that describes the proportion of methylated methylation sites. The subset of methylation sites may be referred to as selected methylation sites. Methylation sites may be selected in certain combinations in both a test cfDNA fragment and corresponding control cfDNA fragments. For example, in some instances, the selected methylation sites are all the methylation sites in the entire length of the test cfDNA fragment, and all the methylation sites in the corresponding control cfDNA fragments. In some instances, the selected methylation sites all the methylation sites in the test cfDNA fragment, and only the methylation sites in corresponding control cfDNA fragments that positionally overlap with the selected methylation sites in the test cfDNA fragment and that align within a common subgenomic interval with the test cfDNA fragment. In some instances, the selected methylation sites are all the methylation sites in the corresponding control cfDNA fragments, and only the methylation sites in a test cfDNA fragment that positionally overlap with the selected methylation sites in the corresponding control cfDNA fragments and that align within a common subgenomic interval with the corresponding control cfDNA fragments. In some instances, the selected methylation sites are only the methylation sites in a test cfDNA fragment that positionally overlap with the selected methylation sites in the corresponding control cfDNA fragments and that align within a common subgenomic interval with the corresponding control cfDNA fragments, and only the methylation sites in corresponding control cfDNA fragments that positionally overlap with the selected methylation sites in the test cfDNA fragment and that align within a common subgenomic interval with the test cfDNA fragment.

At 408 in FIG. 4, a fragment length difference is determined. Each of one or more test cfDNA fragments from the plurality of test cfDNA fragments can have its own fragment length difference. Fragment length differences may be calculated as described above and with reference to FIGS. 1C-1D. The fragment length difference for a test cfDNA fragment indicates the difference between fragment length for the test cfDNA fragment and the average fragment length of corresponding control cfDNA fragments. In some aspects, the fragment length difference is a subtracted difference between the fragment length of a test cfDNA fragment and the average fragment length of corresponding control cfDNA fragments. In some aspects, the fragment length difference is a divided fraction between the fragment length of a test cfDNA fragment and the average fragment length corresponding control cfDNA fragments.

A fragment length difference can be determined for each test cfDNA fragment from a subset of a plurality of test cfDNA fragments, or each test cfDNA fragment from a plurality of cfDNA fragments, comparing the test cfDNA fragment to corresponding control cfDNA fragments. Corresponding control cfDNA fragments are control cfDNA fragments that correspond to a given test cfDNA fragment, within each subgenomic interval of a plurality of subgenomic intervals. As described above, the corresponding control cfDNA fragments to a test cfDNA fragment are the control cfDNA fragments that align or overlap with the test cfDNA fragment in any part. The corresponding control cfDNA fragments may also align with the same subgenomic interval as the test cfDNA fragment. The corresponding control cfDNA fragments may also overlap with the same subgenomic interval as the test cfDNA fragment.

For the purposes of calculating a fragment length difference, as described above, the average fragment length of corresponding control cfDNA fragments is compared to the fragment length of a test cfDNA fragment. For both test and control cfDNA fragments, fragment length may describe the length of the entirety of the fragment or the length of only part of the fragment that aligns or overlaps with a subgenomic interval. For example, the fragment length of the test cfDNA fragment may be the length of the entirety of the fragment, and the average fragment length of the corresponding control cfDNA fragments may be the average of the lengths of the entirety of all the corresponding control cfDNA fragments. Alternatively, the fragment length of the test cfDNA fragment may be the length of only the part of the fragment that aligns or overlaps with a subgenomic interval, and the average fragment length of the corresponding control cfDNA fragments may be the average of the lengths of only the parts of the corresponding control cfDNA fragments that align or overlap with the same subgenomic interval as the test cfDNA fragment.

Filtering the Test cfDNA Fragments

At 410 in FIG. 4, the value, the methylation fraction difference, and/or the fragment length difference for a test cfDNA fragment are input into a trained filter model. This can be done for each of one or more test cfDNA fragments from the plurality of test cfDNA fragments from the test subject. The value, methylation fraction difference, and fragment length difference for each test cfDNA fragment are used as inputs for identifying whether the test cfDNA fragment originated from tissue impacted by disease or not and labeling the test cfDNA fragment accordingly.

At 412 in FIG. 4, the plurality of test cfDNA fragments are filtered using the filter model. Each of one or more test cfDNA fragments from the plurality of test cfDNA fragments can be filtered. The filtering step uses the subgenomic specific-intervals that are set for the trained filter model using the methods of FIG. 2. Sequencing data for test cfDNA fragments can be filtered depending on whether features of the test cfDNA fragments, determined as described above, pass through subgenomic interval-specific thresholds or not. For example, a subgenomic interval-specific threshold may be a numerical value that is a minimum cutoff, and a test cfDNA fragment that passes through the threshold may be one with a feature that is higher than the cutoff value of the threshold. If a test cfDNA fragment passes through a threshold, it may be described as having been filtered.

At 414 in FIG. 4, test cfDNA fragments that pass through a predetermined number of filters in the filter model are labeled as originating from tissue impacted by the disease and test cfDNA fragments that do not are labeled as not originating from tissue impacted by the disease. The number of filters is predetermined, meaning that the number of thresholds that a test cfDNA fragment can pass through to be labeled as disease-associated is known in the trained filter model. For example, in some instances, a given test cfDNA fragment in one subgenomic interval can pass through 3 filters in that subgenomic interval to be labeled as originating from tissue impacted by disease: a value threshold, a methylation fraction difference threshold, and a fragment length difference threshold. In some instances, a given test cfDNA fragment in one subgenomic interval can pass through 2 filters in that subgenomic interval to be labeled as originating from tissue impacted by disease: a value threshold and a methylation fraction difference threshold, a value threshold and a fragment length difference threshold, or a methylation fraction difference threshold and a fragment length difference threshold. In some instances, a given test cfDNA fragment in one subgenomic interval can pass through 1 filter in that subgenomic interval to be labeled as originating from tissue impacted by disease: a value threshold, a methylation fraction difference threshold, or a fragment length difference threshold. In any of the above instances, if a test cfDNA fragment aligns or overlaps with more than one subgenomic interval, it may pass through one or more thresholds in each of the subgenomic intervals that it aligns or overlaps with to be labeled as originating from tissue impacted by disease.

Filtering and labeling the test cfDNA fragments, with reference to blocks 410, 412, and 414 in FIG. 4, can be performed for a plurality of different types of diseases using a number of different trained filter models, wherein the different trained filter models are trained for different types of diseases. In some aspects, filtering and labeling the test cfDNA fragments may be performed for a plurality of different types of cancers using different trained filter models, wherein the different trained filter models are trained for the different types of cancers. In some aspects, filtering and labeling the test cfDNA fragments may be performed for a plurality of different types of cardiovascular or neurological diseases using different trained filter models, wherein the different trained filter models are trained for the different types of cardiovascular or neurological diseases.

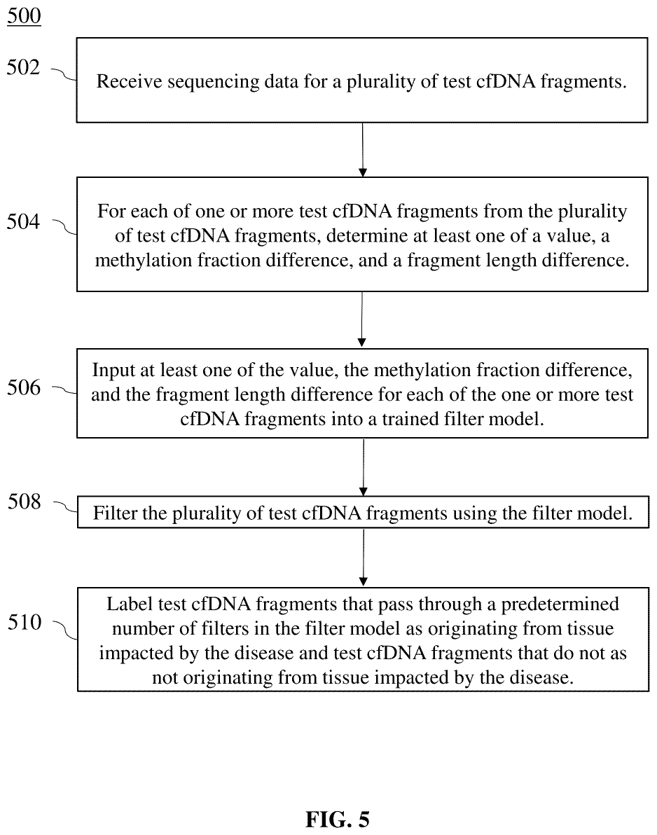

FIG. 5 provides another non-limiting exemplary schematic showing general process 500 for labeling test cfDNA fragments from a test subject. The method of labeling test cfDNA fragments can include: receiving sequencing data for a plurality of test cfDNA fragments (502); for each of one or more test cfDNA fragments from the plurality of test cfDNA fragments, determining at least one of a value, a methylation fraction difference, and a fragment length difference (504); inputting at least one of the value, the methylation fraction difference, and the fragment length difference for each of the one or more test cfDNA fragments into a trained filter model (506); filtering the plurality of test cfDNA fragments using the filter model (508); and labeling test cfDNA fragments that pass through a predetermined number of filters in the filter model as originating from tissue impacted by the disease and test cfDNA fragments that do not as not originating from tissue impacted by the disease (510).

At 502 in FIG. 5, sequencing data is received for cfDNA fragments, as described for block 402 in FIG. 4. At 504 in FIG. 5, at least one feature is extracted from the test cfDNA fragments, following the methods described for blocks 404, 406, and/or 408 in FIG. 4. For each test cfDNA fragment from a plurality of test cfDNA fragments, at least one of a value, methylation fraction difference, fragment length difference can be determined, using the methods described above. The feature can then be used in the trained filter model to label if test cfDNA fragments originated from tissue impacted by disease or not. In some aspects, at least two of a value, a methylation fraction difference, and a fragment length difference are determined. For example, a value and a methylation fraction difference may be determined; a value and a fragment length difference may be determined; or a methylation fraction difference and a fragment length difference may be determined. Alternatively, only one of a value, a methylation fraction difference, or a fragment length difference may be determined; or all three of a value, a methylation fraction difference, or a fragment length difference may be determined. The value may be determined with reference to FIGS. 1A-1B and block 404 in FIG. 4; the methylation fraction difference may be determined with reference to FIGS. 1A-1B and block 406 in FIG. 4; and the fragment length difference may be determined with reference to FIGS. 1C-1D and block 408 in FIG. 4.

At 506 in FIG. 5, at least one of the value, the methylation fraction difference, and the fragment length difference for a test cfDNA fragment are input into a trained filter model. This can be done for each of one or more test cfDNA fragments from the plurality of test cfDNA fragments from the test subject. At least one of the value, methylation fraction difference, and fragment length difference for each test cfDNA fragment can be used as inputs for identifying whether the test cfDNA fragment originated from tissue impacted by disease or not and labeling the test cfDNA fragment accordingly.

At 508 in FIG. 5, the plurality of test cfDNA fragments are filtered using the filter model. Each of one or more test cfDNA fragments from the plurality of test cfDNA fragments can be filtered. The filtering step uses the subgenomic specific-intervals that are set for the trained filter model using the methods of FIG. 3. Sequencing data for test cfDNA fragments can be filtered depending on whether features of the test cfDNA fragments, determined as described above, pass through subgenomic interval-specific thresholds or not. For example, a subgenomic interval-specific threshold may be a numerical value that is a minimum cutoff, and a test cfDNA fragment that passes through the threshold may be one with a feature that is higher than the cutoff value of the threshold. If a test cfDNA fragment passes through a threshold, it may be described as having been filtered.

At 510 in FIG. 5, test cfDNA fragments that pass through a predetermined number of filters in the filter model are labeled as originating from tissue impacted by the disease and test cfDNA fragments that do not are labeled as not originating from tissue impacted by the disease. The predetermined number of filters is determined by the relevant features and subgenomic intervals in the trained filter model that may be used to identify an association with disease or not. The features and subgenomic intervals represent different biological contexts that may be informative about an association with disease. For example, for a given test cfDNA fragment, a predetermined number of filters can be all three of a subgenomic interval-specific value threshold, methylation fraction difference threshold, and fragment length difference threshold, within only one subgenomic interval. Alternatively, for a given test cfDNA fragment, a predetermined number of filters can be all three of a subgenomic interval-specific value threshold, methylation fraction difference threshold, and fragment length difference threshold, within more than one subgenomic interval. Alternatively, for a given test cfDNA fragment, a predetermined number of filters can be at least one of a subgenomic interval-specific value threshold, methylation fraction difference threshold, and fragment length difference threshold, within only one subgenomic interval. Alternatively, for a given test cfDNA fragment, a predetermined number of filters can be at least one of a subgenomic interval-specific value threshold, methylation fraction difference threshold, and fragment length difference threshold, within more than one subgenomic interval.

Filtering and labeling the test cfDNA fragments, with reference to blocks 506, 508, and 510 in FIG. 5, can be performed for a plurality of different types of diseases using a number of different trained filter models, wherein the different trained filter models are trained for different types of diseases. In some aspects, filtering and labeling the test cfDNA fragments may be performed for a plurality of different types of cancers using different trained filter models, wherein the different trained filter models are trained for the different types of cancers. In some aspects, filtering and labeling the test cfDNA fragments may be performed for a plurality of different types of cardiovascular or neurological diseases using different trained filter models, wherein the different trained filter models are trained for the different types of cardiovascular or neurological diseases.

Process 400 or 500 can be performed, for example, using one or more electronic devices implementing a software platform. In some examples, process 400 or 500 is performed using a client-server system, and the blocks of process 400 or 500 are divided up in any manner between the server and a client device. In other examples, the blocks of process 400 or 500 are divided up between the server and multiple client devices. Thus, while portions of process 400 or 500 are described herein as being performed by particular devices of a client-server system, it will be appreciated that process 400 or 500 is not so limited. In other examples, process 400 or 500 is performed using only a client device or only multiple client devices. In process 400 or 500, some blocks are, optionally, combined, the order of some blocks is, optionally, changed, and some blocks are, optionally, omitted. In some examples, additional steps may be performed in combination with the process 400 or 500. Accordingly, the operations as illustrated (and described in greater detail below) are exemplary by nature and, as such, should not be viewed as limiting.

Methods for Generating Test cfDNA Fragment Metrics

Test cfDNA fragment metrics can also be generated for the test cfDNA fragments that have been filtered and labeled as originating from the tissue impacted by the disease. After a test sample from a test subject is analyzed to filter and label certain test cfDNA fragments associated with disease, the filtered test cfDNA fragments that are associated with disease can be analyzed again to generate metrics using a computer-implemented process. The test cfDNA fragment metrics can also describe disease ontologies or disease states.

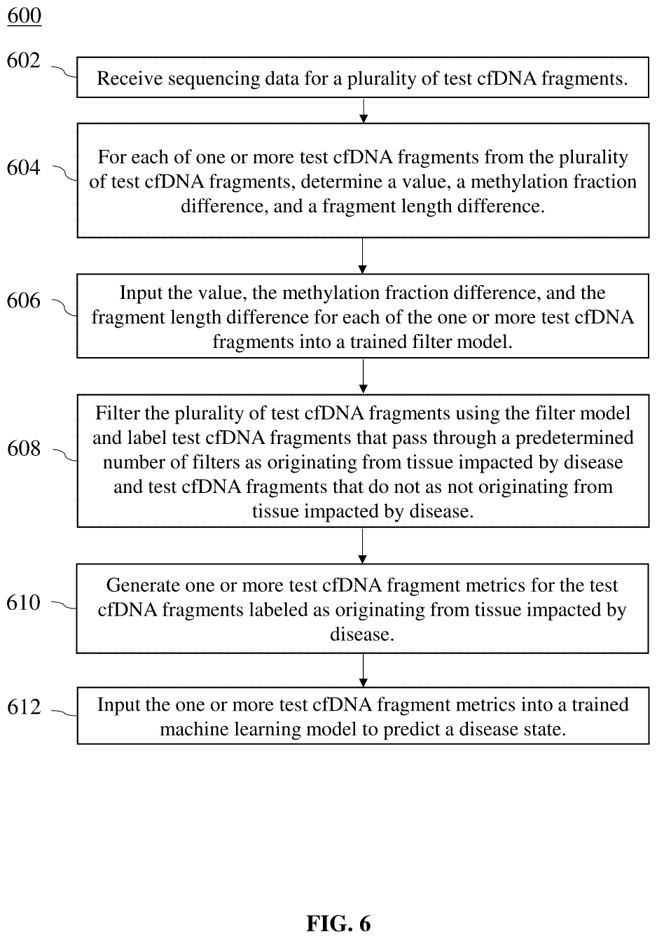

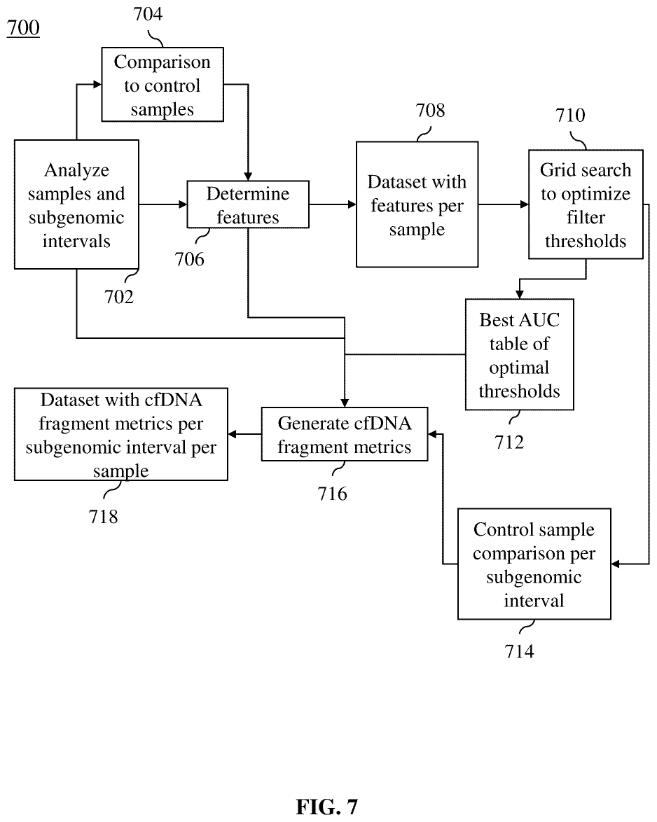

FIG. 6 provides a non-limiting exemplary schematic showing general process 600 for generating test cfDNA fragment metrics. The method of generating test cfDNA fragment metrics can include: receiving sequencing data for a plurality of test cfDNA fragments (602); determining a value, a methylation fraction difference, and a fragment length difference for each test cfDNA fragment (604); inputting the value, the methylation fraction difference, and the fragment length difference for each of the one or more test cfDNA fragments into a trained filter model (606); filtering the plurality of test cfDNA fragments using the filter model and labeling test cfDNA fragments that pass through a predetermined number of filters in the filter model as originating from tissue impacted by disease and test cfDNA fragments that do not as not originating from tissue impacted by disease (608); generating one or more test cfDNA fragment metrics for the test cfDNA fragments labeled as originating from tissue impacted by disease (610); inputting the one or more test cfDNA fragment metrics into a trained machine learning model to predict a disease state (612).

At 602 in FIG. 6, sequencing data is received for cfDNA fragments, as described for block 402 in FIG. 4. At 604 in FIG. 6, features are extracted that describe test cfDNA fragments and corresponding control cfDNA fragments (value, methylation fraction difference, fragment length difference), as described for blocks 404, 406, and 408 in FIG. 4. At 606 in FIG. 6, the extracted features are input into a trained filter model, as described for block 410 in FIG. 4. At 608 in FIG. 6, the test cfDNA fragments are filtered using the thresholds of the trained filter model and labeled as originating from tissue impacted by disease or not, as described for blocks 412 and 414 in FIG. 4.

At 610 in FIG. 6, test cfDNA fragment metrics are generated. Test cfDNA fragment metrics can be calculated in each subgenomic interval. Each subgenomic interval may have one metric, where the metric is a numeric value that provides some information about the test cfDNA fragments that align or overlap with that subgenomic interval. Each subgenomic interval may have multiple metrics. The metrics may be calculated by taking the aggregate of the test cfDNA fragments that align or overlap with that subgenomic interval. The aggregate can be determined in multiple ways. For example, an aggregate may be a fraction of test cfDNA fragments out of all the test cfDNA fragments that align or overlap with that subgenomic interval. An aggregate may be an average of all the test cfDNA fragments that align or overlap with that subgenomic interval. An aggregate may be a total number of test cfDNA fragments that align or overlap with that subgenomic interval having a specific characteristic.

The test cfDNA fragment metrics that may be computed may include, but are not limited to: a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, which may be calculated as a subtracted difference between the average methylation fraction for test cfDNA fragments and the average methylation fraction for control cfDNA fragments, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by a methylation fraction difference of test cfDNA fragments labeled as originating from the tissue impacted by the disease, or a fraction of test cfDNA fragments labeled as originating from the tissue impacted by the disease multiplied by a methylation fraction difference of test cfDNA fragments labeled as originating from the tissue impacted by the disease.

The computed test cfDNA fragment metrics may also include, and are not limited to: various algebraic combinations of the value, methylation fraction difference, and fragment length difference as determined using the steps of the methods provided herein; entropic differences such as H-diff or Kullback-Leibler divergence; or matrix distances computed from a variety of cfDNA fragment feature vectors.

At 612 in FIG. 6, test cfDNA fragment metrics are input into a trained machine learning model. The model can be configured to receive the one or more test cfDNA fragment metrics and predict whether the test subject has disease. For example, the model can predict whether the test subject from which the sequencing data for the test cfDNA fragments was received has a hyperproliferative disease or a non-proliferative disease. The model may be used to predict whether the test subject has a specific type of hyperproliferative disease, such as cancer, and may be used to predict what a specific type of cancer and/or a specific stage of cancer.

FIG. 7 provides another non-limiting exemplary schematic showing general process 700 for generating cfDNA fragment metrics. The method of generating cfDNA fragment metrics can include: analyzing samples and subgenomic intervals (702); comparing to control samples (704); determining features (706); organizing features per sample (708); optimizing filter thresholds via a grid search (710); generating a best AUC table of optimal thresholds (712); determining control sample comparisons per subgenomic interval (714); generating cfDNA fragment metrics (716); organizing cfDNA fragment metrics per subgenomic interval per sample (718).

At 702 in FIG. 7, samples and subgenomic intervals are analyzed. As described for block 202 in FIG. 2, block 302 in FIG. 3, block 402 in FIG. 4, and block 502 in FIG. 5, samples can come from training subjects who are known to have a disease or test subjects who are known to have a disease, are suspected to have a disease, or do not have a disease. The samples can be sequenced to producing sequencing data for cfDNA fragments. The resulting sequencing data for cfDNA fragments from any of these samples can be examined in different subgenomic intervals. The intervals can be genomic regions of varying sizes that represent different biological contexts. This analysis can involve organizing the sequencing data for cfDNA fragments on a per-interval basis in a dataset.

At 704 in FIG. 7, the samples can be compared against control samples, which may be analyzed separately, or together with the samples from block 702. As described for block 202 in FIG. 2, block 302 in FIG. 3, block 402 in FIG. 4, and block 502 in FIG. 5, control samples can come from control subjects who are labeled as not having a disease. These samples can be sequenced to produce sequencing data for control cfDNA fragments. This step can involve organizing the sequencing data from the training samples, test samples, and/or control samples on a per-interval basis in a dataset.

At 706 in FIG. 7, features are determined for the training samples, test samples, and/or control samples. As described for blocks 204, 206, 208 in FIG. 2, block 304 in FIG. 3, blocks 404, 406, and 408 in FIG. 4, and block 504 in FIG. 5, at least one, at least two, or all three of a value, methylation fraction difference, and fragment length difference can be determined for the samples. The features that can be determined at this step can also include metrics that are first computed to subsequently calculate a value, methylation fraction difference, and fragment length difference. For example, the number of methylation sites and the methylation status of methylation sites can be first computed before calculating a value. Methylation fractions can be first computed before calculating a methylation fraction difference. Fragment lengths can be first computed before calculating a fragment length difference. Any of these features can be determined directly from training or test samples (block 702), directly from control samples (block 704), or following a comparison between training or test samples and control samples (blocks 702 and 704).

At 708 in FIG. 7, features are organized into a dataset per sample. This may be done per training sample, per test sample, or per control sample. At 710 in FIG. 7, a grid search is done, as described for block 210 in FIGS. 2 and 306 in FIG. 3. The grid search approach tests different combinations of filter thresholds to select optimal filter thresholds. These optimal filter thresholds can be numerical cutoffs, above which or below which cfDNA fragments can be labeled as disease-associated or not. Data from training samples from subjects who are known to have disease and data from control samples from subjects who are known to not have disease can be analyzed using the grid search approach.

At 712 in FIG. 7, a best AUC table of optimal thresholds is generated. The best AUC table is a product of the grid search approach. The thresholds that provide the highest AUC can be identified as the optimal thresholds for use in the model. In some instances, the best AUCs may be aggregated to identify optimal filter thresholds.

At 714 in FIG. 7, features of control samples (per block 706) are organized as a reference table for generating fragment metrics. For example, during the grid search method, cfDNA fragments from all control samples can be processed to compute fragment length per subgenomic interval. Within one subgenomic interval, this can be the average fragment length of all control cfDNA fragments that align with or positionally overlap that subgenomic interval. The average fragment lengths for control cfDNA fragments in each subgenomic interval can be organized into a reference table. This information can then be used to compute a fragment length difference for each test cfDNA fragment.

At 716 in FIG. 7, cfDNA fragment metrics are generated. These metrics can be determined using a number of different data inputs. The features calculated within training samples, test samples, control samples, or training or test samples compared to control samples can be analyzed to compute cfDNA fragment metrics. The cfDNA fragment metrics can be determined per subgenomic interval, and/or per sample. The optimal thresholds as laid out in the best AUC table can also all be analyzed to compute additional features (and ultimately generate cfDNA fragment metrics). The additional features can be, for example, methylation fraction differences (with the initial features being methylation fractions for training or test samples and control samples) or fragment length differences (with the initial features being fragment lengths for training or test samples and control samples). After computing these additional features, fragments can be filtered and labeled as described for blocks 412 and 414 in FIG. 4, or blocks 508 and 510 in FIG. 5. Once filtered, metrics can be generated as described for block 612 in FIG. 6, providing information about the cfDNA fragments in different subgenomic intervals. At 718 in FIG. 7, the cfDNA fragment metrics can be organized per subgenomic interval per sample.

Process 600 or 700 can be performed, for example, using one or more electronic devices implementing a software platform. In some examples, process 600 or 700 is performed using a client-server system, and the blocks of process 600 or 700 are divided up in any manner between the server and a client device. In other examples, the blocks of process 600 or 700 are divided up between the server and multiple client devices. Thus, while portions of process 600 or 700 are described herein as being performed by particular devices of a client-server system, it will be appreciated that process 600 or 700 is not so limited. In other examples, process 600 or 700 is performed using only a client device or only multiple client devices. In process 600 or 700, some blocks are, optionally, combined, the order of some blocks is, optionally, changed, and some blocks are, optionally, omitted. In some examples, additional steps may be performed in combination with the process 600 or 700. Accordingly, the operations as illustrated (and described in greater detail below) are exemplary by nature and, as such, should not be viewed as limiting.

### Methods of Use

In some instances, the disclosed methods may further comprise one or more of the steps of: (i) obtaining the sample from the subject (e.g., a subject suspected of having or determined to have disease), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain features related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a liquid biopsy sample or a normal control. In some instances, the sample may be a liquid biopsy sample and may comprise blood, plasma, cerebrospinal fluid, sputum, stool, urine, or saliva. In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for training a filter model for filtering and labeling test cfDNA fragments from a test sample from a test subject may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for training a filter model for filtering and labeling test cfDNA fragments from a test sample from a test subject may be used to select a subject (e.g., a patient) for a clinical trial based on the classification of test cfDNA fragments as associated with disease or not, the type or stage of disease, or the metrics computed for test cfDNA fragments labeled as associated with disease. In some instances, patient selection for clinical trials based on, e.g., the classification of test cfDNA fragments as associated with disease or not, the type or stage of disease, or the metrics computed for test cfDNA fragments labeled as associated with disease, may accelerate the development of targeted therapies and improve the healthcare outcomes for treatment decisions.

In some instances, the disclosed methods for training a filter model for filtering and labeling test cfDNA fragments from a test sample from a test subject may be used to select an appropriate therapy or treatment (e.g., an anti-cancer therapy or anti-cancer treatment) for a subject. For example, the disclosed methods can be used to determine a most likely type of disease or most likely type of cancer for a test subject using a trained filter model specific to a certain type of disease or a certain type of cancer. Responsive to the determination of the most likely type of disease or most likely type of cancer for a test subject, an appropriate therapy or treatment may be selected for the test subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for training a filter model for filtering and labeling test cfDNA fragments from a test sample from a test subject may be used in treating a disease (e.g., a cancer) in a subject. For example, in response to determining the most likely type of disease using any of the methods disclosed herein, an effective amount of an anti-cancer therapy or anti-cancer treatment may be administered to the subject.

In some instances, the disclosed methods for training a filter model for filtering and labeling test cfDNA fragments from a test sample from a test subject may be used for monitoring disease progression or recurrence (e.g., cancer or tumor progression or recurrence) in a subject. For example, in some instances, the methods may be used to determine the type or stage of disease as determined by a trained filter model for test cfDNA fragments in a first sample obtained from the subject at a first time point, and used to determine the type or stage of disease as determined by a trained filter model for test cfDNA fragments in a second sample obtained from the subject at a second time point, where comparison of the first determination of disease type or stage and the second determination of disease type or stage allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the type or stage of disease as determined by a trained filter model for test cfDNA fragments.

In some instances, the one or more test cfDNA fragment metrics determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for filtering and labeling test cfDNA fragments may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for filtering and labeling test cfDNA fragments as part of a genomic profiling process (or inclusion of the output from the disclosed methods for filtering and labeling test cfDNA fragments as part of the genomic profile of the subject) can improve the validity of, e.g., disease detection calls and treatment decisions, made on the basis of the genomic profile by, for example, independently confirming the presence of disease-specific variants, such as cancer-specific variants in a given sample from a subject.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (NMR) pathway.

### Samples

The disclosed methods and systems may be used with any of a variety of training samples, test samples, or control samples (also referred to herein as specimens) comprising nucleic acids (e.g., DNA) that are collected from a subject (e.g., a patient). Examples of a sample include, but are not limited to, a tumor sample, a biopsy sample (e.g., a liquid biopsy), a blood sample (e.g., a peripheral whole blood sample), a blood plasma sample, a blood serum sample, a lymph sample, a saliva sample, a sputum sample, a urine sample, a gynecological fluid sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a hematologic malignancy or pre-malignancy. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal sample). In some instances, the disclosed methods may further comprise determining if a primary control is available and, if so, isolating a control nucleic acid (e.g., DNA) from said primary control. In some instances, the sample may comprise any normal control (e.g., a normal adjacent liquid (NAL)) if no primary control is available. In some instances, the sample may be or may comprise histologically normal liquid sample. In some instances, the method includes evaluating a sample, e.g., a histologically normal liquid sample using the methods described herein. In some instances, the disclosed methods may further comprise acquiring a sub-sample enriched for non-tumor cells, e.g., by macro-dissecting a non-tumor liquid sample from said NAL in a sample not accompanied by a primary control. In some instances, the disclosed methods may further comprise determining that no primary control and no NAL is available, and marking said sample for analysis without a matched control.

In some instances, samples obtained from histologically normal liquid samples may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of liquid samples (or disease states thereof), e.g., liquid biopsy samples. Liquid samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of less than 5%, at least 5-50%, at least 10-40%, at least 15-25%, or at least 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA), e.g., from a liquid sample or from a normal liquid sample. In certain instances, the sample may further comprise a non-nucleic acid component, e.g., cells, protein, carbohydrate, or lipid, e.g., from the liquid sample or normal liquid sample.

### Subjects

In some instances, the training sample is obtained (e.g., collected) from a training subject (e.g., patient) known to have a condition or disease (e.g., a hyperproliferative disease or a non-cancer indication). In some instances, the test sample is obtained (e.g., collected) from a test subject (e.g., patient) known to have a condition or disease, suspected of having a condition or disease, unknown to have a condition or disease, or not having a condition or disease (e.g., a hyperproliferative disease or a non-cancer indication). In some instances, the hyperproliferative disease is a cancer. In some instances, the cancer is a solid tumor or a metastatic form thereof. In some instances, the cancer is a hematological cancer, e.g., a leukemia or lymphoma. In some instances, the control sample is obtained (e.g., collected) from a control subject (e.g., patient) known to not have a condition or disease.

In some instances, the training subject or test subject has a cancer or is at risk of having a cancer. For example, in some instances, the training or test subject has a genetic predisposition to a cancer (e.g., having a genetic mutation that increases his or her baseline risk for developing a cancer). In some instances, the training or test subject has been exposed to an environmental perturbation (e.g., radiation or a chemical) that increases his or her risk for developing a cancer. In some instances, the training or test subject is in need of being monitored for development of a cancer. In some instances, the training or test subject is in need of being monitored for cancer progression or regression, e.g., after being treated with an anti-cancer therapy (or anti-cancer treatment). In some instances, the training or test subject is in need of being monitored for relapse of cancer. In some instances, the training or test subject is in need of being monitored for minimum residual disease (MRD). In some instances, the training or test subject has been, or is being treated, for cancer. In some instances, the training or test subject has not been treated with an anti-cancer therapy (or anti-cancer treatment).

In some instances, the training or test subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the training or test sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA may be extracted from liquid biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). A typical DNA extraction procedure, for example, comprises (i) collection of the fluid sample, cell sample, or tissue sample from which DNA is to be extracted, (ii) treatment of the fluid sample or lysed sample with a concentrated salt solution to precipitate proteins, lipids, and RNA, followed by centrifugation to separate out the precipitated proteins, lipids, and RNA, and (iii) purification of DNA from the supernatant to remove detergents, proteins, salts, or other reagents used during the cell membrane lysis step.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Cytosine Conversion

Methylation patterns in nucleic acid molecules from a sample from a subject may be converted into converted nucleic acids via cytosine conversion (also referred to herein as “nucleotide conversion”, “methylation conversion”, and simply “conversion”). As part of this conversion, methylation patterns of interest that may be present in the nucleic acid molecules in the sample from the subject are converted into nucleic acid patterns of interest corresponding to the genomic locus. Performing a methylation conversion reaction may comprise, e.g., selectively converting non-methylated (also referred to herein as unmethylated) cytosine residues to uracil residues, or selectively converting methylated cytosine residues to uracil residues. Cytosine residues that have been converted into uracil residues are then further converted to thymine residues by, e.g., amplifying converted nucleic acids. Thus, amplified converted nucleic acids (that contain, e.g., thymine residues in positions in which the nucleic acid molecules in the sample from the subject had, e.g., unmethylated cytosine residues), may also be referred to as converted nucleic acids.

The conversion step converts a methylation state or a methylation pattern into a methylation signal or methylation status, as the converted nucleic acids may not retain actual methylated residues but do comprise information about the methylation pattern that was present in the pre-converted nucleotides. Thus, converted nucleic acids are representative of a methylation state in that they comprise patterns associated with a methylation state.

Conversion may be performed by various methods known in the art, such as, e.g., a bisulfite reaction (see, e.g., Li et al. (2011), “DNA Methylation Detection: Bisulfite Genomic Sequencing Analysis”, Methods Mol. Biol. 791:11-21), enzymatic conversion reactions, or the like. For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu et al. (2019), “Bisulfite-Free Direct Detection of 5-Methylcytosine and 5-Hydroxymethylcytosine at Base Resolution”, Nature Biotechnology, vol. 37, pp. 424-429). In some embodiments, converting comprises TET-assisted bisulfite treatment, TET-assisted pyridine borane treatment, oxidative bisulfite treatment, APOBEC treatment, and/or other DNA deaminase treatment (e.g., as discussed in Vaisvila et al. (2023), “Discovery of novel DNA cytosine deaminase activities enables a nondestructive single-enzyme methylation sequencing method for base resolution high-coverage methylome mapping of cell-free and ultra-low input DNA”, bioRxiv, 2023.06.29.547047).

In some embodiments, conversion comprises enzymatic conversion. In some embodiments, conversion comprises bisulfite conversion. In some embodiments, the conversion comprises cytosine (C) to thymine (T) conversion. In some embodiments, the conversion converts non-methylated cytosine residues into thymine residues. In some embodiments, the conversion converts methylated cytosine residues into thymine residues.

In some embodiments, the conversion converts non-methylated cytosine residues into uracil residues. In some embodiments, the conversion converts methylated cytosine residues into uracil residues. In some embodiments, the conversion converts methylated cytosine residues into dihydrouracil residues. In some embodiments, the conversion converts non-methylated cytosine residues into dihydrouracil residues.

Bisulfite conversion or treatment refers to a biochemical process for converting unmethylated cytosine residue to uracil or thymine residues (e.g., deamination to uracil, followed by amplification as thymine during PCR), whereby methylated cytosine residues (e.g., 5-methylcytosine, 5mC; or 5-hydroxymethylcytosine, 5hmC) are preserved. Reagents to convert cytosine to uracil are known to those of skill in the art and include bisulfite reagents such as sodium bisulfite, potassium bisulfite, ammonium bisulfite, magnesium bisulfite, sodium metabisulfite, potassium metabisulfite, ammonium metabisulfite, magnesium metabisulfite and the like.

In some embodiments, the methods of the present disclosure comprise treating a plurality of nucleic acids or nucleic acid fragments of the present disclosure with enzymatic digestion and bisulfite treatment. The principle of the method is that the fragmentation of DNA is not achieved by ultrasound but achieved by combined enzymatic digestion by multiple endonucleases (MseI, Tsp 509I, NlaIII and Hpy CH4V), wherein the restriction enzyme cutting sites of MseI, Tsp509I, NlaIII and Hpy CH4V are TTAA, AATT, CATG and TGCA, respectively. See, e.g., Smiraglia D J et al. Oncogene 2002; 21: 5414-5426. This is followed by bisulfite treatment, e.g., as described herein. In some embodiments, fragmentation of DNA is derived from the biological processes that shed DNA into the bloodstream to create cfDNA.

Enzymatic methods for cytosine conversion are also known, e.g., enzymatic methyl sequencing (EM-seq). Such approaches can be advantageous because they employ enzymes instead of bisulfite, which can damage and fragment DNA, leading to DNA loss and potentially biased sequencing. For example, TET2 (the Ten-eleven translocation (Tet) family 2 methylcytosine dioxygenase) and T4-BGT (T4 phage beta-glucosyltransferase) can be used to convert 5mC and 5hmC into products that cannot be deaminated by APOBEC3A (apolipoprotein B mRNA editing enzyme, catalytic polypeptide-like 3A), then APOBEC3A is used to deaminate unmodified cytosines by converting them into uracils. See, e.g., Vaisvila, R. et al. (2021) Genome Res. 31:1-10.

In some embodiments, the methods of the present disclosure comprise treating a plurality of nucleic acids or nucleic acid fragments of the present disclosure with TET-assisted bisulfite (e.g., TAB-seq). In the TAB-seq approach, beta-glucosyltransferase (βGT) is used to convert 5hmC into β-glucosyl-5-hydroxymethylcytosine (5gmC), and a Tet enzyme (e.g., mTet1) is used to oxidize 5mC into 5-carboxylcytosine (5caC). Subsequently, nucleic acids can be treated with bisulfite. See, e.g., Yu, M. et al. (2018) Methods Mol. Biol. 1708:645-663.

In some embodiments, the methods of the present disclosure comprise treating a plurality of nucleic acids or nucleic acid fragments of the present disclosure with TET-assisted pyridine borane (e.g., TAPS). In the TAPS approach, a TET methylcytosine dioxygenase is used to oxidize 5mC and 5hmC into 5caC, then 5caC is reduced into dihydrouracil (DHU) via pyridine borane. DHU is converted to thymine during subsequent PCR. See, e.g., Liu, Y. et al. (2019) Nat. Biotechnol. 37:424-429.

In some embodiments, the methods of the present disclosure comprise treating a plurality of nucleic acids or nucleic acid fragments of the present disclosure with oxidative bisulfite (e.g., oxBS). In the oxBS approach, 5hmC is oxidized into 5-formylcytosine (5fC), which can be converted to uracil under bisulfite. Sequencing results from bisulfite vs. oxidative bisulfite treatment can then be used to infer 5hmC levels from 5mC. See, e.g., Booth, M. J. et al. (2013) Nat. Protocols 8:1841-1851. This approach can be scaled on a genome-wide level in oxBS-seq; see, e.g., Kirschner, K. et al. (2018) Methods Mol. Biol. 1708:665-678.

In some embodiments, the methods of the present disclosure comprise treating a plurality of nucleic acids or nucleic acid fragments of the present disclosure with APOBEC. Enzymatic reagents to convert cytosine to uracil, i.e. cytosine deaminases, include those of the APOBEC family, such as APOBEC-seq or APOBEC3A. The APOBEC family members are cytidine deaminases that convert cytosine to uracil while maintaining 5-methyl cytosine, i.e. without altering 5-methyl cytosine. Such enzymes are described in US2013/0244237 and WO2018165366 and are commercially available (see, e.g., the NEBNext® Enzymatic Methyl-seq Kit, New England Biolabs). Non-limiting examples of APOBEC family proteins include APOBEC1, APOBEC2, APOBEC3A, APOBEC3B, APOBEC3C, APOBEC3D, APOBEC3F, APOBEC3G, APOBEC3H, APOBEC4, and Activation-induced (cytidine) deaminase.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS platform. In some instances, sequencing may comprise Illumina MiSeqÔ sequencing. In some instances, sequencing may comprise Illumina HiSeq® sequencing. In some instances, sequencing may comprise Illumina NovaSeq® sequencing or other similar sequencers, for example, sequencing with an Illumina NovaSeq X system or with an Illumina NovaSeq 6000 system. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci. In some instances, the methods disclosed herein may be applied to multiple subgenomic intervals based on the sequence reads that are acquired. For example, the disclosed methods may be applied to at least about 1,000, at least about 5,000, at least about 10,000, at least about 20,000, at least about 50,000, at least about 75,000, at least about 100,000, at least about 125,000, at least about 150,000, at least about 175,000, or at least about 200,000 subgenomic intervals. In some embodiments, the disclosed methods may be applied to between about 100,000 subgenomic intervals to about 150,000 subgenomic intervals.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein comprise cfDNA fragments that align with subgenomic intervals. Included within the scope of this type of alignment are cfDNA fragments that align with or within a subgenomic interval, and also cfDNA fragments that overlap with the subgenomic interval. For example, this type of alignment includes cfDNA fragments that fall entirely within the subgenomic interval. Additionally, this type of alignment also includes cfDNA fragments that overlap the edges of the subgenomic interval. Additionally, this type of alignment also includes cfDNA fragments that fall entirely within the subgenomic interval and overlap the edges of the subgenomic interval.

In some instances, the methods disclosed herein comprise selected methylation sites that are selected based on alignment within a common subgenomic interval, or selected cfDNA fragments that are selected based on alignment within a subgenomic interval. Included within the scope of this type of alignment are methylation sites or cfDNA fragments that align with or within a subgenomic interval, and also methylation sites or cfDNA fragments that overlap with the subgenomic interval. For example, this type of alignment includes methylation sites or cfDNA fragments that fall entirely within the subgenomic interval. Additionally, this type of alignment also includes methylation sites or cfDNA fragments that overlap the edges of the subgenomic interval. Additionally, this type of alignment also includes methylation sites or cfDNA fragments that fall entirely within the subgenomic interval and overlap the edges of the subgenomic interval.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for training a filter model for filtering cfDNA fragments from sample sequencing data obtained from a training sample of a training subject having a disease, and labeling test cfDNA fragments from a test sample from a test subject. The systems may comprise, e.g., one or more processors, and a memory unit communicatively coupled to the one or more processors and configured to store instructions that, when executed by the one or more processors, cause the system to: receive, at one or more processors, sample sequencing data for a plurality of sample cfDNA fragments; determine, using the one or more processors, for each of the one or more sample cfDNA fragments from the plurality of sample cfDNA fragments, at least one of: a value indicating that the sample cfDNA fragment originated from tissue not impacted by a disease; a methylation fraction difference between a methylation fraction for corresponding control cfDNA fragments and a methylation fraction for the sample cfDNA fragment; a fragment length difference between an average length of corresponding control cfDNA fragments and the fragment length of the sample cfDNA fragment; determine, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and set, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds. Alternatively, the systems may comprise, e.g., one or more processors, and a memory unit communicatively coupled to the one or more processors and configured to store instructions that, when executed by the one or more processors, cause the system to: receive, at one or more processors, test sequencing data for a plurality of test cfDNA fragments; determine, using the one or more processors, for each of one or more test cfDNA fragments from the plurality of test cfDNA fragments, at least one of: a value indicating that the test cfDNA fragment originated from tissue not impacted by the disease; a methylation fraction difference between a methylation fraction for corresponding control cfDNA fragments and a methylation fraction for the test cfDNA fragment; a fragment length difference between an average length of corresponding control cfDNA fragments and a fragment length of the test cfDNA fragment; input, using the one or more processors, for each of the one or more test cfDNA fragments, at least one of the value, the methylation fraction difference, and the fragment length difference into a trained filter model; filter, using the one or more processors, the plurality of test cfDNA fragments using the filter model; and label, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for training a filter model for filtering cfDNA fragments extracted from any of a variety of samples as described herein (e.g., a tissue sample, biopsy sample, hematological sample, or liquid biopsy sample derived from the subject), or for labeling test cfDNA fragments extracted from any of a variety of samples as described herein (e.g., a tissue sample, biopsy sample, hematological sample, or liquid biopsy sample derived from the subject).

In some instances, the plurality of genomic loci (e.g., CpG sites) for which sequencing data is processed to determine a methylation state may comprise at least 1, 2, 3, 4, 5, 6, 7, 8, 9, 10, 20, 30, 40, 50, 60, 70, 80, 90, 100, 200, 300, 400, 500, 600, 700, 800, 900, 1000, or more than 1000 genomic loci (or any number of genomic loci within the range of 1 to more than 1000 genomic loci).

In some instances, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the filtering and/or labeling of test cfDNA fragments is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, ƒ, where ƒ may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

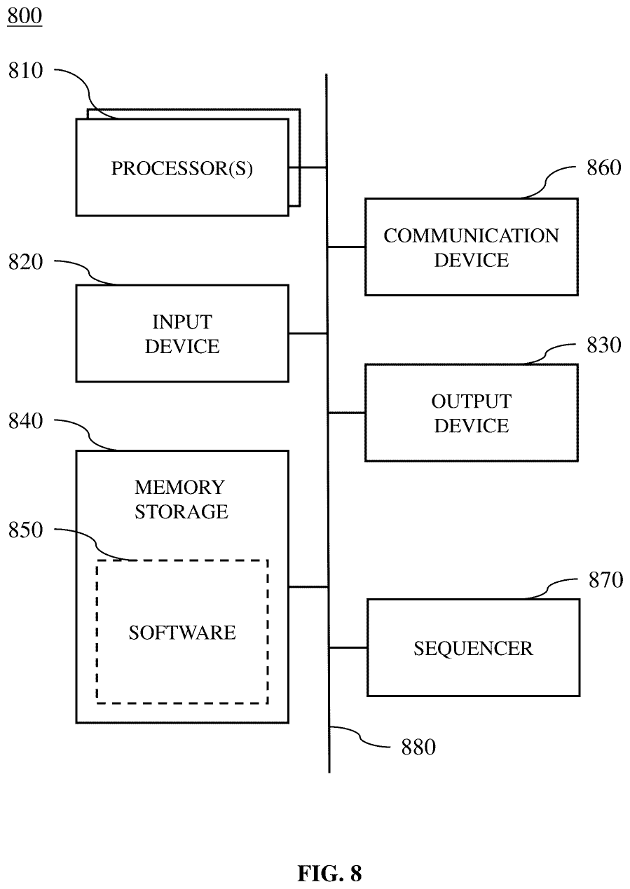

FIG. 8 illustrates an example of a computing device or system in accordance with one embodiment. Device 800 can be a host computer connected to a network. Device 800 can be a client computer or a server. As shown in FIG. 8, device 800 can be any suitable type of microprocessor-based device, such as a personal computer, workstation, server or handheld computing device (portable electronic device) such as a phone or tablet. The device can include, for example, one or more processor(s) 810, input devices 820, output devices 830, memory or storage devices 840, communication devices 860, and nucleic acid sequencers 870. Software 850 residing in memory or storage device 840 may comprise, e.g., an operating system as well as software for executing the methods described herein. Input device 820 and output device 830 can generally correspond to those described herein, and can either be connectable or integrated with the computer.

Input device 820 can be any suitable device that provides input, such as a touch screen, keyboard or keypad, mouse, or voice-recognition device. Output device 830 can be any suitable device that provides output, such as a touch screen, haptics device, or speaker.

Storage 840 can be any suitable device that provides storage (e.g., an electrical, magnetic or optical memory including a RAM (volatile and non-volatile), cache, hard drive, or removable storage disk). Communication device 860 can include any suitable device capable of transmitting and receiving signals over a network, such as a network interface chip or device. The components of the computer can be connected in any suitable manner, such as via a wired media (e.g., a physical system bus 880, Ethernet connection, or any other wire transfer technology) or wirelessly (e.g., Bluetooth®, Wi-Fi®, or any other wireless technology).

Software module 850, which can be stored as executable instructions in storage 840 and executed by processor(s) 810, can include, for example, an operating system and/or the processes that embody the functionality of the methods of the present disclosure (e.g., as embodied in the devices as described herein).

Software module 850 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 840, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

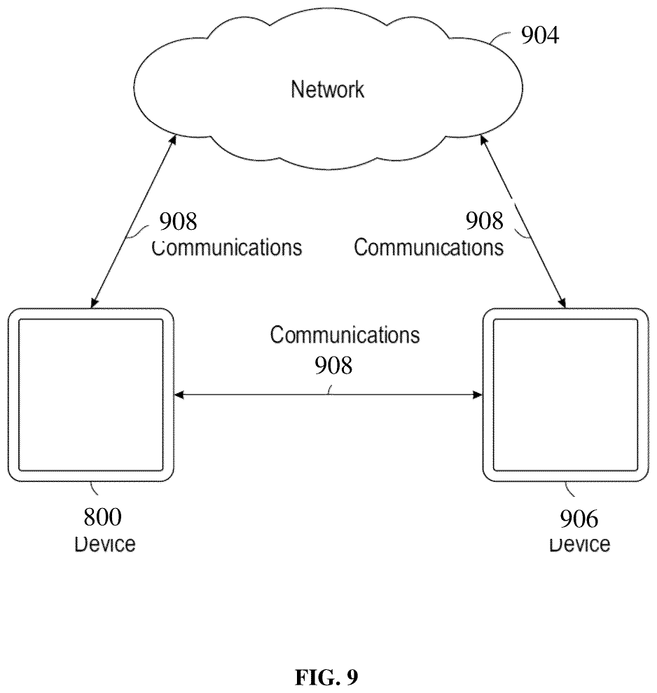

Device 800 may be connected to a network (e.g., network 1004, as shown in FIG. 9 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 800 can be implemented using any operating system, e.g., an operating system suitable for operating on the network. Software module 850 can be written in any suitable programming language, such as C, C++, Java, Python, or R. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 810.

Device 800 can further include a sequencer 870, which can be any suitable nucleic acid sequencing instrument.

FIG. 9 illustrates an example of a computing system in accordance with one embodiment. In system 900, device 800 (e.g., as described above and illustrated in FIG. 8) is connected to network 904, which is also connected to device 906. In some embodiments, device 906 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, Pacific Biosciences' PacBio® RS system, Ultima Genomics UG 90™ platform, or the Illumina NovaSeq X series platform.

Devices 800 and 906 may communicate, e.g., using suitable communication interfaces via network 904, such as a Local Area Network (LAN), Virtual Private Network (VPN), or the Internet. In some embodiments, network 904 can be, for example, the Internet, an intranet, a virtual private network, a cloud network, a wired network, or a wireless network. Devices 800 and 906 may communicate, in part or in whole, via wireless or hardwired communications, such as Ethernet, IEEE 802.11b wireless, or the like. Additionally, devices 800 and 906 may communicate, e.g., using suitable communication interfaces, via a second network, such as a mobile/cellular network. Communication between devices 800 and 906 may further include or communicate with various servers such as a mail server, mobile server, media server, telephone server, and the like. In some embodiments, Devices 800 and 906 can communicate directly (instead of, or in addition to, communicating via network 904), e.g., via wireless or hardwired communications, such as Ethernet, IEEE 802.11b wireless, or the like. In some embodiments, devices 800 and 906 communicate via communications 908, which can be a direct connection or can occur via a network (e.g., network 904).

One or all of devices 800 and 906 generally include logic (e.g., http web server logic) or are programmed to format data, accessed from local or remote databases or other sources of data and content, for providing and/or receiving information via network 904 according to various examples described herein.

### Exemplary Implementations

Exemplary implementations of the methods and systems described herein include:

1. A method of training a filter model for filtering cfDNA fragments, comprising:
receiving, at one or more processors, sample sequencing data for a plurality of sample cfDNA fragments;
determining, using the one or more processors, for each of one or more sample cfDNA fragments from the plurality of sample cfDNA fragments, at least one of:
   a value indicating that the sample cfDNA fragment originated from tissue not impacted by a disease;
   a methylation fraction difference between a methylation fraction for corresponding control cfDNA fragments and a methylation fraction for the sample cfDNA fragment, and
   a fragment length difference between an average length of corresponding control cfDNA fragments and a fragment length of the sample cfDNA fragment;
determining, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and
setting, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

2. The method of clause 1, wherein the plurality of sample cfDNA fragments are obtained from a training sample of a training subject having a disease.

3. The method of clause 1 or 2, further comprising:
determining, using the one or more processors, for each of one or more sample cfDNA fragments from the plurality of sample cfDNA fragments, at least two of:
a value indicating that the sample cfDNA fragment originated from tissue not impacted by a disease;
a methylation fraction difference between a methylation fraction for corresponding control cfDNA fragments and a methylation fraction for the sample cfDNA fragment, and
a fragment length difference between an average length of corresponding control cfDNA fragments and a fragment length of the sample cfDNA fragment.

4. The method of any one of clauses 1-3, further comprising:
determining, using the one or more processors, for each of a plurality of subgenomic intervals, at least two of a subgenomic interval-specific value threshold, a subgenomic interval-specific methylation fraction difference threshold, and a subgenomic interval-specific fragment length difference threshold.

5. The method of any one of clauses 1-4, further comprising:
setting, using the one or more processors, one or more thresholds of a filter model based on at least two of the determined subgenomic interval-specific value threshold, the determined subgenomic interval-specific methylation fraction difference threshold, and the determined subgenomic interval-specific fragment length threshold.

6. The method of any one of clauses 1-5, wherein the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length.

7. The method of clause 6, wherein the value is a statistical value indicating the probability that the sample cfDNA fragment originated from tissue not impacted by a disease, and wherein the statistical value is based on the methylation status for the one or more first selected methylation sites in the sample cfDNA fragment and a methylation status for one or more third selected methylation sites in corresponding control cfDNA fragments.

8. The method of clause 6 or 7, wherein the methylation fraction difference is between the methylation fraction for one or more fourth selected methylation sites in the corresponding control cfDNA fragments and the methylation fraction for one or more second selected methylation sites in the sample cfDNA fragment.

9. A method of training a filter model for filtering cfDNA fragments, comprising:
(a) receiving, at one or more processors, sample sequencing data for a plurality of sample cfDNA fragments obtained from a training sample of a training subject having a disease, the sample sequencing data comprising, for each of one or more sample cfDNA fragments from the plurality of sample cfDNA fragments, a methylation status for one or more first selected methylation sites, a methylation fraction for one or more second selected methylation sites, and a fragment length;
(b) generating, using the one or more processors, for each of the one or more sample cfDNA fragments, a value indicating that the sample cfDNA fragment originated from tissue not impacted by the disease based on the methylation status for the one or more first selected methylation sites in the sample cfDNA fragment and a methylation status for one or more third selected methylation sites in corresponding control cfDNA fragments;
(c) determining, using the one or more processors, for each of the one or more sample cfDNA fragments, a methylation fraction difference between a methylation fraction for one or more fourth selected methylation sites in corresponding control cfDNA fragments and the methylation fraction for the one or more second selected methylation sites in the sample cfDNA fragment;
(d) determining, using the one or more processors, for each of the one or more sample cfDNA fragments, a fragment length difference between an average length of corresponding control cfDNA fragments and the fragment length of the sample cfDNA fragment;
(e) determining, using the one or more processors, for each of a plurality of subgenomic intervals, a subgenomic interval-specific value threshold, a subgenomic interval-specific methylation fraction difference threshold, and a subgenomic interval-specific fragment length difference threshold based on the values, methylation fraction differences, and fragment length differences of each of the one or more sample cfDNA fragments that align with the subgenomic interval; and
(f) setting, using the one or more processors, one or more thresholds of a filter model using the determined subgenomic interval-specific value thresholds, the determined subgenomic interval-specific methylation fraction difference thresholds, and the determined subgenomic interval-specific fragment length thresholds.

10. The method of clause 9, wherein the value is a statistical value indicating the probability that the sample cfDNA fragment originated from tissue not impacted by the disease.

11. The method of any one of clauses 6-10, wherein the one or more first selected methylation sites are all the methylation sites in the sample cfDNA fragment, and the one or more third selected methylation sites are all the methylation sites in the corresponding control cfDNA fragments.

12. The method of any one of clauses 6-10, wherein the one or more first selected methylation sites are all the methylation sites in a sample cfDNA fragment, and the one or more third selected methylation sites are selected based on a positional overlap with the one or more first selected methylation sites in the sample cfDNA fragment and alignment within a common subgenomic interval with the sample cfDNA fragment.

13. The method of any one of clauses 6-10, wherein the one or more third selected methylation sites are all the methylation sites in the corresponding control cfDNA fragments, and the one or more first selected methylation sites are selected based on a positional overlap with the one or more third selected methylation sites in the corresponding control cfDNA fragments and alignment within a common subgenomic interval with the corresponding control cfDNA fragments.

14. The method of any one of clauses 6-10, wherein the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment.

15. The method of any one of clauses 6-14, wherein the one or more second selected methylation sites are all the methylation sites in the sample cfDNA fragment, and the one or more fourth selected methylation sites are all the methylation sites in the corresponding control cfDNA fragments.

16. The method of any one of clauses 6-14, wherein the one or more second selected methylation sites are all the methylation sites in a sample cfDNA fragment, and the one or more fourth selected methylation sites are selected based on a positional overlap with the one or more second selected methylation sites in the sample cfDNA fragment and alignment within a common subgenomic interval with the sample cfDNA fragment.

17. The method of any one of clauses 6-14, wherein the one or more fourth selected methylation sites are all the methylation sites in the corresponding control cfDNA fragments, and the one or more second selected methylation sites are selected based on a positional overlap with the one or more fourth selected methylation sites in the corresponding control cfDNA fragments and alignment within a common subgenomic interval with the corresponding control cfDNA fragments.

18. The method of any one of clauses 6-14, wherein the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment. 19. A method of labeling test cfDNA fragments from a test subject, comprising:
receiving, at one or more processors, test sequencing data for a plurality of test cfDNA fragments;
determining, using the one or more processors, for each of one or more test cfDNA fragments from the plurality of test cfDNA fragments, at least one of:
   a value indicating that the test cfDNA fragment originated from tissue not impacted by the disease;
   a methylation fraction difference between a methylation fraction for corresponding control cfDNA fragments and a methylation fraction for the test cfDNA fragment;
   a fragment length difference between an average length of corresponding control cfDNA fragments and a fragment length of the test cfDNA fragment;
inputting, using the one or more processors, for each of the one or more test cfDNA fragments, at least one of the value, the methylation fraction difference, and the fragment length difference into a trained filter model;
filtering, using the one or more processors, the plurality of test cfDNA fragments using the filter model; and
labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

20. The method of clause 19, wherein the plurality of test cfDNA fragments are obtained from a test sample of a test subject.

21. The method of clause 19 or 20, wherein the selected corresponding control cfDNA fragments are selected based on alignment within a common subgenomic interval with the test cfDNA fragment.

22. The method of any one of clauses 19-21, further comprising:
determining, using the one or more processors, for each of one or more test cfDNA fragments from the plurality of test cfDNA fragments, at least two of:
   a value indicating that the test cfDNA fragment originated from tissue not impacted by a disease;
   a methylation fraction difference between a methylation fraction for corresponding control cfDNA fragments and a methylation fraction for the test cfDNA fragment, and
   a fragment length difference between an average length of corresponding control cfDNA fragments and a fragment length of the test cfDNA fragment.

23. The method of any one of clauses 19-22, further comprising:
determining, using the one or more processors, for each of a plurality of subgenomic intervals, at least two of a subgenomic interval-specific value threshold, a subgenomic interval-specific methylation fraction difference threshold, and a subgenomic interval-specific fragment length difference threshold.

24. The method of any one of clauses 19-23, further comprising:
setting, using the one or more processors, one or more thresholds of a filter model based on at least two of the determined subgenomic interval-specific value threshold, the determined subgenomic interval-specific methylation fraction difference threshold, and the determined subgenomic interval-specific fragment length threshold.

25. The method of any one of clauses 19-24, wherein the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length.

26. The method of clause 25, wherein the value is a statistical value indicating the probability that the sample cfDNA fragment originated from tissue not impacted by a disease, and wherein the statistical value is based on the methylation status for the one or more first selected methylation sites in the test cfDNA fragment and a methylation status for one or more third selected methylation sites in corresponding control cfDNA fragments.

27. The method of clause 25 or 26, wherein the methylation fraction difference is between the methylation fraction for one or more fourth selected methylation sites in the corresponding control cfDNA fragments and the methylation fraction for one or more second selected methylation sites in the test cfDNA fragment.

28. A method of labeling test cfDNA fragments from a test subject, comprising:
receiving, at one or more processors, test sequencing data comprising, for each of one or more test cfDNA fragments from a plurality of test cfDNA fragments from a test sample from the test subject, a methylation status for one or more first selected methylation sites, a methylation fraction for one or more second selected methylation sites, and a fragment length;
generating, using the one or more processors, for each of the one or more test cfDNA fragments, a value indicating that the test cfDNA fragment originated from tissue not impacted by the disease based on the methylation status for the one or more first selected methylation sites in the test cfDNA fragment and a methylation status for one or more third selected methylation sites in corresponding control cfDNA fragments;
determining, using the one or more processors, for each of the one or more test cfDNA fragments, a methylation fraction difference between a methylation fraction for one or more fourth selected methylation sites in the corresponding control cfDNA fragments and the methylation fraction for the one or more second selected methylation sites in the test cfDNA fragment;
determining, using the one or more processors, for each of the one or more test cfDNA fragments, a fragment length difference between an average length of corresponding control cfDNA fragments and the fragment length of the test cfDNA fragment;
   inputting, using the one or more processors, for each of the one or more test cfDNA fragments, the value, the methylation fraction difference, and the fragment length difference into a trained filter model;
   filtering, using the one or more processors, the plurality of test cfDNA fragments using the filter model; and
labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

29. The method of clause 28, wherein the value is a statistical value indicating the probability that the test cfDNA fragment originated from tissue not impacted by the disease.

30. The method of any one of clauses 25-29, wherein the one or more first selected methylation sites are all the methylation sites in the test cfDNA fragment, and the one or more third selected methylation sites are all the methylation sites in the corresponding control cfDNA fragments.

31. The method of any one of clauses 25-29, wherein the one or more first selected methylation sites are all the methylation sites in a test cfDNA fragment, and the one or more third selected methylation sites are selected based on a positional overlap with the one or more first selected methylation sites in the test cfDNA fragment and alignment within a common subgenomic interval with the test cfDNA fragment.

32. The method of any one of clauses 25-29, wherein the one or more third selected methylation sites are all the methylation sites in the corresponding control cfDNA fragments, and the one or more first selected methylation sites are selected based on a positional overlap with the one or more third selected methylation sites in the corresponding control cfDNA fragments and alignment within a common subgenomic interval with the corresponding control cfDNA fragments.

33. The method of any one of clauses 25-29, wherein the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment.

34. The method of any one of clauses 25-33, wherein the one or more second selected methylation sites are all the methylation sites in the test cfDNA fragment, and the one or more fourth selected methylation sites are all the methylation sites in the corresponding control cfDNA fragments.

35. The method of any one of clauses 25-33, wherein the one or more second selected methylation sites are all the methylation sites in a test cfDNA fragment, and the one or more fourth selected methylation sites are selected based on a positional overlap with the one or more second selected methylation sites in the test cfDNA fragment and alignment within a common subgenomic interval with the test cfDNA fragment.

36. The method of any one of clauses 25-33, wherein the one or more fourth selected methylation sites are all the methylation sites in the corresponding control cfDNA fragments, and the one or more second selected methylation sites are selected based on a positional overlap with the one or more fourth selected methylation sites in the corresponding control cfDNA fragments and alignment within a common subgenomic interval with the corresponding control cfDNA fragments.

37. The method of any one of clauses 25-33, wherein the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment.

38. The method of any one of clauses 19-37, comprising sequencing the test cfDNA fragments to obtain the test sequencing data.

39. The method of clause 38, wherein the test sequencing data was obtained by:
providing a plurality of test cfDNA fragments obtained from a test sample from a test subject;
ligating one or more adapters onto one or more test cfDNA fragments from the plurality of test cfDNA fragments;
amplifying the one or more ligated test cfDNA fragments from the plurality of test cfDNA fragments;
capturing amplified test cfDNA fragments from the amplified test cfDNA fragments;
sequencing, by a sequencer, the captured test cfDNA fragments to obtain a plurality of sequence reads that represent the captured test cfDNA fragments.

40. The method of clause 39, wherein the captured test cfDNA fragments are captured from the amplified test cfDNA fragments by hybridization to one or more bait molecules.

41. The method of clause 40, wherein the one or more bait molecules comprise one or more cfDNA fragments, each comprising a region that is complementary to a region of a captured test cfDNA fragment.

42. The method of any one of clauses 26-41, wherein generating the statistical value comprises:
aligning, using the one or more processors, the test cfDNA fragment and the plurality of corresponding control cfDNA fragments to a reference sequence to generate an alignment;
identifying from the alignment, using the one or more processors, corresponding control cfDNA fragments that positionally overlap with the test cfDNA fragment; and
comparing, using the one or more processors, the methylation status for the one or more first selected methylation sites in the test cfDNA fragment to the methylation status for the one or more third selected methylation sites in the corresponding control cfDNA fragments.

43. The method of clause 42, wherein comparing the methylation status for the one or more first selected methylation sites in the test cfDNA fragment to the methylation status for one or more third selected methylation sites in the corresponding control cfDNA fragments comprises determining a number of methylated sites in the one or more first selected methylation sites in the test cfDNA fragment, a number of unmethylated sites in the one or more first selected methylation sites in the test cfDNA fragment, a number of methylated sites in the one or more third selected methylation sites in the corresponding control cfDNA fragments, and a number of unmethylated sites in the one or more third selected methylation sites in the corresponding control cfDNA fragments.

44. The method of any one of clauses 26-43, wherein a binomial test is used to generate the statistical value for each of the test cfDNA fragments from the plurality of test cfDNA fragments.

45. The method of any one of clauses 19-44, further comprising generating one or more test cfDNA fragment metrics for the test cfDNA fragments labeled as originating from the tissue impacted by the disease.

46. The method of clause 45, wherein the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease.

47. The method of clause 45 or 46, comprising inputting the one or more test cfDNA fragment metrics into a trained machine learning model configured to receive the one or more test cfDNA fragment metrics and predict whether the test subject has the disease.

48. The method of any one of clauses 19-47, wherein the test subject is suspected of having or is determined to have the disease.

49. The method of any one of clauses 19-48, wherein the method is repeated for a plurality of different types of diseases using different trained filter models, wherein the different trained filter models are trained for the different types of diseases.

50. The method of any one of clauses 19-49, wherein the method is performed for a plurality of different types of cancers using different trained filter models, wherein the different trained filter models are trained for the different types of cancers.

51. The method of clause 49 or 50, further comprising determining a most likely type of disease or most likely type of cancer for the test subject.

52. The method of any one of clauses 19-51, wherein the method is performed for a plurality of different types of cardiovascular or neurological diseases using different trained filter models, wherein the different trained filter models are trained for the different types of cardiovascular or neurological diseases.

53. The method of clause 49 or 52, further comprising determining a most likely type of cardiovascular or neurological disease for the test subject.

54. A method for diagnosing a disease, comprising:
determining the most likely type of disease or the most likely type of cancer for the test subject according to the method of clause 51; and
diagnosing the test subject as having the disease based on the determination of the most likely type of disease or the most likely type of cancer for the test subject.

55. A method of selecting an anti-cancer therapy, comprising:
determining the most likely type of cancer for the test subject according to the method of clause 51; and
responsive to the determination of the most likely type of cancer for the test subject, selecting an anti-cancer therapy for the test subject.

56. A method of treating a cancer in a subject, comprising:
determining the most likely type of cancer for the test subject according to the method of clause 51; and
responsive to determining the most likely type of cancer for the test subject, administering an effective amount of an anti-cancer therapy to the subject.

57. The method of clause 55 or 56, wherein the anti-cancer therapy comprises a targeted anti-cancer therapy.

58. The method of clause 57, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

59. The method of clause 57 or 58, wherein the anti-cancer therapy comprises resection, chemotherapy, radiation, or increased screening.

60. The method of any one of clauses 19-59, wherein the trained filter model was previously trained according to a method comprising:
(a) receiving, at one or more processors, sample sequencing data for a plurality of sample cfDNA fragments obtained from a training sample of a training subject having a disease, the sample sequencing data comprising, for each of one or more sample cfDNA fragments from the plurality of sample cfDNA fragments, a methylation status for one or more first selected methylation sites, a methylation fraction for one or more second selected methylation sites, and a fragment length;
(b) generating, using the one or more processors, for each of the one or more sample cfDNA fragments, a value indicating that the sample cfDNA fragment originated from tissue not impacted by the disease based on the methylation status for the one or more first selected methylation sites in the sample cfDNA fragment and a methylation status for one or more third selected methylation sites in corresponding control cfDNA fragments;
(c) determining, using the one or more processors, for each of the one or more sample cfDNA fragments, a methylation fraction difference between a methylation fraction for one or more fourth selected methylation sites in the corresponding control cfDNA fragments and the methylation fraction for the one or more second selected methylation sites in the sample cfDNA fragment;
(d) determining, using the one or more processors, for each of the one or more sample cfDNA fragments, a fragment length difference between an average length of corresponding control cfDNA fragments and the fragment length of the sample cfDNA fragment;
(e) determining, using the one or more processors, for each of a plurality of subgenomic intervals, a subgenomic interval-specific value threshold, a subgenomic interval-specific methylation fraction difference threshold, and a subgenomic interval-specific fragment length difference threshold based on the values, methylation fraction differences, and fragment length differences of each of the one or more sample cfDNA fragments that align with the subgenomic interval; and
(f) setting, using the one or more processors, one or more thresholds of a filter model using the determined subgenomic interval-specific value thresholds, the determined subgenomic interval-specific methylation fraction difference thresholds, and the determined subgenomic interval-specific fragment length thresholds.

61. The method of any one of clauses 1-18 or clause 60, comprising sequencing the sample cfDNA fragments to obtain the sample sequencing data.

62. The method of clause 61, wherein the sample sequencing data was obtained by:
providing a plurality of sample cfDNA fragments obtained from a training sample from a training subject;
ligating one or more adapters onto one or more sample cfDNA fragments from the plurality of sample cfDNA fragments;
amplifying the one or more ligated sample cfDNA fragments from the plurality of sample cfDNA fragments;
capturing amplified sample cfDNA fragments from the amplified sample cfDNA fragments;
sequencing, by a sequencer, the captured sample cfDNA fragments to obtain a plurality of sequence reads that represent the captured sample cfDNA fragments.

63. The method of clause 62, wherein the captured sample cfDNA fragments are captured from the amplified sample cfDNA fragments by hybridization to one or more bait molecules.

64. The method of clause 63, wherein the one or more bait molecules comprise one or more cfDNA fragments, each comprising a region that is complementary to a region of a captured sample cfDNA fragment.

65. The method of any one of clauses 38-41 or 46-49, wherein the sequencing comprises use of a massively parallel sequencing (MPS) technique, whole genome sequencing (WGS), whole exome sequencing, targeted sequencing, direct sequencing, or Sanger sequencing technique.

66. The method of clause 65, wherein the sequencing comprises massively parallel sequencing, and the massively parallel sequencing technique comprises next generation sequencing (NGS).

67. The method of any one of clauses 1-66, wherein the one or more first selected methylation sites, the one or more second selected methylation sites, the one or more third selected methylation sites, and the one or more fourth selected methylation sites are CpG sites in a reference sequence.

68. The method of any one of clauses 1-67, wherein the methylation status for the one or more first selected methylation sites comprises a number of methylated methylation sites and a number of unmethylated methylation sites, the methylation status for the one or more second selected methylation sites comprises a number of methylated methylation sites and a number of unmethylated methylation sites, the methylation status for the one or more third selected methylation sites comprises a number of methylated methylation sites and a number of unmethylated methylation sites, and the methylation status for the one or more fourth selected methylation sites comprises a number of methylated methylation sites and a number of unmethylated methylation sites.

69. The method of any one of clauses 1-68, wherein the value is a p-value.

70. The method of any one of clauses 1-18 or 60-69, wherein generating the value comprises: aligning, using the one or more processors, the sample cfDNA fragment and the plurality of corresponding control cfDNA fragments to a reference sequence to generate an alignment, identifying from the alignment, using the one or more processors, corresponding control cfDNA fragments that positionally overlap with the sample cfDNA fragment, and comparing, using the one or more processors, the methylation status for the one or more first selected methylation sites in the sample cfDNA fragment to the methylation status for the one or more third selected methylation sites in the corresponding control cfDNA fragment.

71. The method of clause 70, wherein comparing the methylation status for the one or more first selected methylation sites in the sample cfDNA fragment to the methylation status for one or more third selected methylation sites in the corresponding control cfDNA fragments comprises determining a number of methylated sites in the one or more first selected methylation sites in the sample cfDNA fragment, a number of unmethylated sites in the one or more first selected methylation sites in the sample cfDNA fragment, a number of methylated sites in the one or more third selected methylation sites in the corresponding control cfDNA fragments, and a number of unmethylated sites in the one or more third selected methylation sites in the corresponding control cfDNA fragments.

72. The method of any one of clauses 1-18 or 60-71, wherein a binomial test is used to generate the value for each of the sample cfDNA fragments from the plurality of sample cfDNA fragments.

73. The method of any one of clauses 1-72, wherein determining the subgenomic interval-specific value threshold, the subgenomic interval-specific methylation fraction difference threshold, and the subgenomic interval-specific fragment length difference threshold comprises:
determining a plurality of receiver operating characteristic (ROC) curves using a plurality of different subgenomic interval-specific value thresholds, a plurality of different subgenomic interval-specific methylation fraction difference thresholds, and a plurality of different subgenomic interval-specific fragment length difference thresholds; and
selecting the subgenomic interval-specific value threshold, the subgenomic interval-specific methylation fraction difference threshold, and the subgenomic interval-specific fragment length difference threshold providing the highest area under the curve (AUC) among the plurality of ROC curves.

74. The method of any one of clauses 1-73, wherein the corresponding control cfDNA fragments were obtained from control subjects labeled as not having the disease.

75. The method of any one of clauses 1-74, wherein the disease is a cancer.

76. The method of clause 75, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

77. The method of clause 76, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

78. The method of any one of clauses 1-77, further comprising obtaining the test sample from the test subject or the training sample from the training subject.

79. The method of any one of clauses 1-78, wherein the test sample or the training sample comprises a tissue biopsy sample.

80. The method of any one of clauses 1-79, wherein the test sample or the training sample is a liquid biopsy sample and comprises blood, plasma, cerebrospinal fluid, sputum, stool, urine, or saliva.

81. The method of any one of clauses 1-80, wherein the cfDNA fragments or a portion thereof comprises circulating tumor DNA (ctDNA) fragments.

82. The method of any one of clauses 1-81, wherein the plurality of subgenomic intervals comprises between 10 and 20 subgenomic intervals, between 10 and 40 subgenomic intervals, between 10 and 60 subgenomic intervals, between 10 and 80 subgenomic intervals, between 10 and 100 subgenomic intervals, between 10 and 150 subgenomic intervals, between 10 and 200 subgenomic intervals, between 10 and 250 subgenomic intervals, between 10 and 300 subgenomic intervals, between 10 and 350 subgenomic intervals, between 10 and 400 subgenomic intervals, between 10 and 450 subgenomic intervals, between 10 and 500 subgenomic intervals, between 20 and 40 subgenomic intervals, between 20 and 60 subgenomic intervals, between 20 and 80 subgenomic intervals, between 20 and 100 subgenomic intervals, between 20 and 150 subgenomic intervals, between 20 and 200 subgenomic intervals, between 20 and 250 subgenomic intervals, between 20 and 300 subgenomic intervals, between 20 and 350 subgenomic intervals, between 20 and 400 subgenomic intervals, between 20 and 500 subgenomic intervals, between 40 and 60 subgenomic intervals, between 40 and 80 subgenomic intervals, between 40 and 100 subgenomic intervals, between 40 and 150 subgenomic intervals, between 40 and 200 subgenomic intervals, between 40 and 250 subgenomic intervals, between 40 and 300 subgenomic intervals, between 40 and 350 subgenomic intervals, between 40 and 400 subgenomic intervals, between 40 and 500 subgenomic intervals, between 60 and 80 subgenomic intervals, between 60 and 100 subgenomic intervals, between 60 and 150 subgenomic intervals, between 60 and 200 subgenomic intervals, between 60 and 250 subgenomic intervals, between 60 and 300 subgenomic intervals, between 60 and 350 subgenomic intervals, between 60 and 400 subgenomic intervals, between 60 and 500 subgenomic intervals, between 80 and 100 subgenomic intervals, between 80 and 150 subgenomic intervals, between 80 and 200 subgenomic intervals, between 80 and 250 subgenomic intervals, between 80 and 300 subgenomic intervals, between 80 and 350 subgenomic intervals, between 80 and 400 subgenomic intervals, between 80 and 500 subgenomic intervals, between 100 and 150 subgenomic intervals, between 100 and 200 subgenomic intervals, between 100 and 250 subgenomic intervals, between 100 and 300 subgenomic intervals, between 100 and 350 subgenomic intervals, between 100 and 400 subgenomic intervals, between 100 and 500 subgenomic intervals, between 150 and 200 subgenomic intervals, between 150 and 250 subgenomic intervals, between 150 and 300 subgenomic intervals, between 150 and 350 subgenomic intervals, between 150 and 400 subgenomic intervals, between 150 and 500 subgenomic intervals, between 200 and 250 subgenomic intervals, between 200 and 300 subgenomic intervals, between 200 and 350 subgenomic intervals, between 200 and 400 subgenomic intervals, between 200 and 500 subgenomic intervals, between 250 and 300 subgenomic intervals, between 250 and 350 subgenomic intervals, between 250 and 400 subgenomic intervals, between 250 and 500 subgenomic intervals, between 300 and 350 subgenomic intervals, between 300 and 400 subgenomic intervals, between 300 and 500 subgenomic intervals, between 350 and 400 subgenomic intervals, between 350 and 500 subgenomic intervals, or between 400 and 500 subgenomic intervals.

83. The method of any one of clauses 1-82, further comprising generating, by the one or more processors, a report indicating the determined subgenomic interval-specific value thresholds, the determined subgenomic interval-specific methylation fraction difference thresholds, the determined subgenomic interval-specific fragment length thresholds, a list of test cfDNA fragments labeled as originating from tissue impacted by the disease and cfDNA fragments, a list of test cfDNA fragments labeled as not originating from tissue impacted by the disease and cfDNA fragments, and/or one or more test cfDNA fragment metrics for the test cfNDA fragments.

84. The method of clause 83, further comprising transmitting the report to a healthcare provider.

85. The method of clause 84, wherein the report is transmitted via a computer network or a peer-to-peer connection.

86. A system comprising:
one or more processors; and
a memory communicatively coupled to the one or more processors and configured to store instructions that, when executed by the one or more processors, cause the system to:
receive, at one or more processors, sample sequencing data for a plurality of sample cfDNA fragments;
determine, using the one or more processors, for each of the one or more sample cfDNA fragments from the plurality of sample cfDNA fragments, at least one of:
   a value indicating that the sample cfDNA fragment originated from tissue not impacted by a disease;
   a methylation fraction difference between a methylation fraction for corresponding control cfDNA fragments and a methylation fraction for the sample cfDNA fragment;
   a fragment length difference between an average length of corresponding control cfDNA fragments and the fragment length of the sample cfDNA fragment;
determine, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and
set, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

87. The system of clause 86, wherein the plurality of sample cfDNA fragments are obtained from a training sample of a training subject having a disease.

88. The system of clause 86 or 87, wherein the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length.

89. The system of clause 88, wherein the value is a statistical value indicating the probability that the sample cfDNA fragment originated from tissue not impacted by a disease, and wherein the statistical value is based on the methylation status for the one or more first selected methylation sites in the sample cfDNA fragment and a methylation status for one or more third selected methylation sites in corresponding control cfDNA fragments.

90. The system of clause 88 or 89, wherein the methylation fraction difference is between the methylation fraction for one or more fourth selected methylation sites in the corresponding control cfDNA fragments and the methylation fraction for one or more second selected methylation sites in the sample cfDNA fragment.

91. The system of any one of clauses 88-90, wherein the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment.

92. The system of any one of clauses 88-91, wherein the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment.

93. A system comprising:
one or more processors; and
a memory communicatively coupled to the one or more processors and configured to store instructions that, when executed by the one or more processors, cause the system to:
receive, at one or more processors, test sequencing data for a plurality of test cfDNA fragments;
determine, using the one or more processors, for each of one or more test cfDNA fragments from the plurality of test cfDNA fragments, at least one of:
   a value indicating that the test cfDNA fragment originated from tissue not impacted by the disease;
   a methylation fraction difference between a methylation fraction for corresponding control cfDNA fragments and a methylation fraction for the test cfDNA fragment;
   a fragment length difference between an average length of corresponding control cfDNA fragments and a fragment length of the test cfDNA fragment;
input, using the one or more processors, for each of the one or more test cfDNA fragments, at least one of the value, the methylation fraction difference, and the fragment length difference into a trained filter model;
filter, using the one or more processors, the plurality of test cfDNA fragments using the filter model; and
label, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

94. The system of clause 93, wherein the plurality of test cfDNA fragments are obtained from a test sample of a test subject.

95. The system of clause 93 or 94, wherein the selected corresponding control cfDNA fragments are selected based on alignment within a common subgenomic interval with the test cfDNA fragment.

96. The system of any one of clauses 93-95, wherein the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length.

97. The system of clause 96, wherein the value is a statistical value indicating the probability that the sample cfDNA fragment originated from tissue not impacted by a disease, and wherein the statistical value is based on the methylation status for the one or more first selected methylation sites in the test cfDNA fragment and a methylation status for one or more third selected methylation sites in corresponding control cfDNA fragments.

98. The system of clause 96 or 97, wherein the methylation fraction difference is between the methylation fraction for one or more fourth selected methylation sites in the corresponding control cfDNA fragments and the methylation fraction for one or more second selected methylation sites in the test cfDNA fragment.

99. The system of any one of clauses 96-98, wherein the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment.

100. The system of any one of clauses 96-99, wherein the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment.

101. The system of any one of clauses 93-100, wherein the instructions, when executed by the one or more processors, further cause the system to generate one or more test cfDNA fragment metrics for the test cfDNA fragments labeled as originating from the tissue impacted by the disease.

102. The system of clause 101, wherein the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease.

103. The system of clause 101 or 102, wherein the instructions, when executed by the one or more processors, further cause the system to input the one or more test cfDNA fragment metrics into a trained machine learning model configured to receive the one or more test cfDNA fragment metrics and predict whether the test subject has the disease.

104. The system of any one of clauses 93-103, wherein the instructions, when executed by the one or more processors, further cause the system to label, using the one or more processors, the test cfDNA fragments for a plurality of different types of diseases or different types of cancers using different trained filter models, wherein the different trained filter models are trained for the different types of diseases or different types of cancers.

105. The system of any one of clauses 93-104, wherein the instructions, when executed by the one or more processors, further cause the system to determine a most likely type of disease or most likely type of cancer for the test subject.

106. The system of any one of clauses 93-105, wherein the trained filter model was previously trained using the system of clause 86.

107. The system of any one of clauses 86-106, further comprising a sequencer.

108. A non-transitory computer-readable storage medium storing one or more programs, the one or more programs comprising instructions, which when executed by one or more processors of a system, cause the system to:
receive, at one or more processors, sample sequencing data for a plurality of sample cfDNA fragments;
determine, using the one or more processors, for each of one or more sample cfDNA fragments from the plurality of sample cfDNA fragments, at least one of:
   a value indicating that the sample cfDNA fragment originated from tissue not impacted by a disease;
   a methylation fraction difference between a methylation fraction for corresponding control cfDNA fragments and a methylation fraction for the sample cfDNA fragment, and
   a fragment length difference between an average length of corresponding control cfDNA fragments and a fragment length of the sample cfDNA fragment;
determine, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and
set, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

109. The non-transitory computer-readable storage medium of clause 108, wherein the plurality of sample cfDNA fragments are obtained from a training sample of a training subject having a disease.

110. The non-transitory computer-readable storage medium of clause 108 or 109, wherein the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length.

111. The non-transitory computer-readable storage medium of clause 110, wherein the value is a statistical value indicating the probability that the sample cfDNA fragment originated from tissue not impacted by a disease, and wherein the statistical value is based on the methylation status for the one or more first selected methylation sites in the sample cfDNA fragment and a methylation status for one or more third selected methylation sites in corresponding control cfDNA fragments.

112. The non-transitory computer-readable storage medium of clause 110 or 111, wherein the methylation fraction difference is between the methylation fraction for one or more fourth selected methylation sites in the corresponding control cfDNA fragments and the methylation fraction for one or more second selected methylation sites in the sample cfDNA fragment.

113. The non-transitory computer-readable storage medium of any one of clauses 110-112, wherein the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment.

114. The non-transitory computer-readable storage medium of any one of clauses 110-113, wherein the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment.

115. A non-transitory computer-readable storage medium storing one or more programs, the one or more programs comprising instructions, which when executed by one or more processors of a system, cause the system to:
receive, at one or more processors, test sequencing data for a plurality of test cfDNA fragments;
determine, using the one or more processors, for each of one or more test cfDNA fragments from the plurality of test cfDNA fragments, at least one of:
   a value indicating that the test cfDNA fragment originated from tissue not impacted by the disease;
   a methylation fraction difference between a methylation fraction for corresponding control cfDNA fragments and a methylation fraction for the test cfDNA fragment;
   a fragment length difference between an average length of corresponding control cfDNA fragments and a fragment length of the test cfDNA fragment;
input, using the one or more processors, for each of the one or more test cfDNA fragments, at least one of the value, the methylation fraction difference, and the fragment length difference into a trained filter model;
filter, using the one or more processors, the plurality of test cfDNA fragments using the filter model; and
label, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

116. The non-transitory computer-readable storage medium of clause 115, wherein the plurality of test cfDNA fragments are obtained from a test sample of a test subject.

117. The non-transitory computer-readable storage medium of clause 115 or 116, wherein the selected corresponding control cfDNA fragments are selected based on alignment within a common subgenomic interval with the test cfDNA fragment.

118. The non-transitory computer-readable storage medium of any one of clauses 115-117, wherein the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length.

119. The non-transitory computer-readable storage medium of clause 118, wherein the value is a statistical value indicating the probability that the sample cfDNA fragment originated from tissue not impacted by a disease, and wherein the statistical value is based on the methylation status for the one or more first selected methylation sites in the test cfDNA fragment and a methylation status for one or more third selected methylation sites in corresponding control cfDNA fragments.

120. The non-transitory computer-readable storage medium of clause 118 or 119, wherein the methylation fraction difference is between the methylation fraction for one or more fourth selected methylation sites in the corresponding control cfDNA fragments and the methylation fraction for one or more second selected methylation sites in the test cfDNA fragment.

121. The non-transitory computer-readable storage medium of any one of clauses 118-120, wherein the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment.

122. The non-transitory computer-readable storage medium of any one of clauses 118-121, wherein the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment.

123. The non-transitory computer-readable storage medium of any one of clauses 115-122, wherein the instructions, when executed by the one or more processors, further cause the system to generate one or more test cfDNA fragment metrics for the test cfDNA fragments labeled as originating from the tissue impacted by the disease.

124. The non-transitory computer-readable storage medium of clause 123, wherein the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1—Calculating Features of Sample cfDNA Fragments

A liquid biopsy sample from a subject with stage 1 colorectal cancer was sequenced to obtain sequencing data for sample cfDNA fragments. Control samples from control subjects without colorectal cancer were sequenced to obtain sequencing data for control cfDNA fragments. Comparisons were made between sample cfDNA fragments and corresponding control cfDNA fragments to calculate features for each sample cfDNA fragment: a p-value, a methylation fraction difference, and a fragment length difference. Control cfDNA fragments that correspond to each sample cfDNA fragment were determined as described below for each feature.

### P-Value

A p-value was generated for each sample cfDNA fragment using a binomial test that indicated a probability that the sample cfDNA fragment originated from tissue not impacted by disease. Each sample cfDNA fragment and the control cfDNA fragments were aligned to a reference genome sequence to generate an alignment, corresponding control cfDNA fragments that positionally overlapped with each sample cfDNA fragment within the same subgenomic interval were identified, and the methylation status for selected methylation sites (i.e., CpG sites) in the sample cfDNA fragment was compared to the methylation status for selected methylation sites in the corresponding control cfDNA fragments.

The selected methylation sites in the sample cfDNA fragment were the methylation sites that positionally overlapped with methylation sites in corresponding control cfDNA fragments by alignment to the same reference genome sequence, and also that aligned within the same subgenomic interval as the corresponding control cfDNA fragments. The selected methylation sites in the corresponding control cfDNA fragments were the methylation sites that positionally overlapped with methylation sites in the sample cfDNA fragment by alignment to the same reference genome sequence, and also that aligned within the same subgenomic interval as the sample cfDNA fragment. For this comparison, the chromosomal positions of methylation sites (i.e., CpG sites) were determined first for the sample cfDNA fragment under test in a given subgenomic interval, then, the methylation status of those CpG sites was compared to the methylation status of corresponding CpG sites in the corresponding control cfDNA fragments.

The methylation status for selected methylation sites in each sample cfDNA fragment was compared to the methylation status for selected methylation sites in the corresponding control cfDNA fragments using a binomial test to generate a p-value. A p-value was generated using the steps described above for every sample cfDNA fragment within the sample from the subject.

### Methylation Fraction Difference

A methylation fraction difference was determined for each sample cfDNA fragment. Each sample cfDNA fragment and the control cfDNA fragments were aligned to a reference genome sequence to generate an alignment, corresponding control cfDNA fragments that positionally overlapped with each sample cfDNA fragment within the same subgenomic interval were identified, and the methylation fraction for selected methylation sites (i.e., CpG sites) in the sample cfDNA fragment was compared to the methylation fraction for selected methylation sites in the corresponding control cfDNA fragments.

The selected methylation sites in the sample cfDNA fragment were the methylation sites that positionally overlapped with methylation sites in corresponding control cfDNA fragments by alignment to the same reference genome sequence, and also that aligned within the same subgenomic interval as the corresponding control cfDNA fragments. The selected methylation sites in the corresponding control cfDNA fragments were the methylation sites that positionally overlapped with methylation sites in the sample cfDNA fragment by alignment to the same reference genome sequence, and also that aligned within the same subgenomic interval as the sample cfDNA fragment. For this comparison, the chromosomal positions of methylation sites (i.e., CpG sites) were determined first for the sample cfDNA fragment under test in a given subgenomic interval, then, the methylation status of those CpG sites was compared to the methylation status of corresponding CpG sites in the corresponding control cfDNA fragments.

The methylation fraction for corresponding control cfDNA fragments was calculated as the number of methylated sites in the selected methylation sites in the corresponding control cfDNA fragments compared to the total number of methylated and unmethylated sites in the selected methylation sites in the corresponding control cfDNA fragments. The methylation fraction for each sample cfDNA fragment was calculated as the number of methylated sites in the selected methylation sites in the sample cfDNA fragment compared to the total number of methylated and unmethylated sites in the selected methylation sites in the sample cfDNA fragment.

The methylation fraction for each sample cfDNA fragment was subtracted from the methylation fraction for corresponding control cfDNA fragments, to generate a methylation fraction difference. A methylation fraction difference was generated using the steps described above for every sample cfDNA fragment within the sample from the subject.

### Fragment Length Difference

A fragment length difference was determined for each sample cfDNA fragment. Each sample cfDNA fragment and the control cfDNA fragments were aligned to a reference genome sequence to generate an alignment, corresponding control cfDNA fragments that positionally overlapped with each sample cfDNA fragment within the same subgenomic interval were identified, and the fragment length of the sample cfDNA fragment was compared to the average fragment length of the corresponding control cfDNA fragments.

The fragment length of each sample cfDNA fragment was calculated as the number of nucleotide positions in the fragment. The average fragment length of corresponding control cfDNA fragments was calculated as the mean of the fragment lengths of all of the control cfDNA fragments in the subset of corresponding control cfDNA fragments that aligned with the same subgenomic interval as the sample cfDNA fragment.

The average fragment length of the corresponding control cfDNA fragments was subtracted from the fragment length of each sample cfDNA fragment, then, the absolute value was taken, generating a fragment length difference per sample cfDNA fragment. A fragment length difference was generated using the steps described above for every sample cfDNA fragment within the sample from the subject.

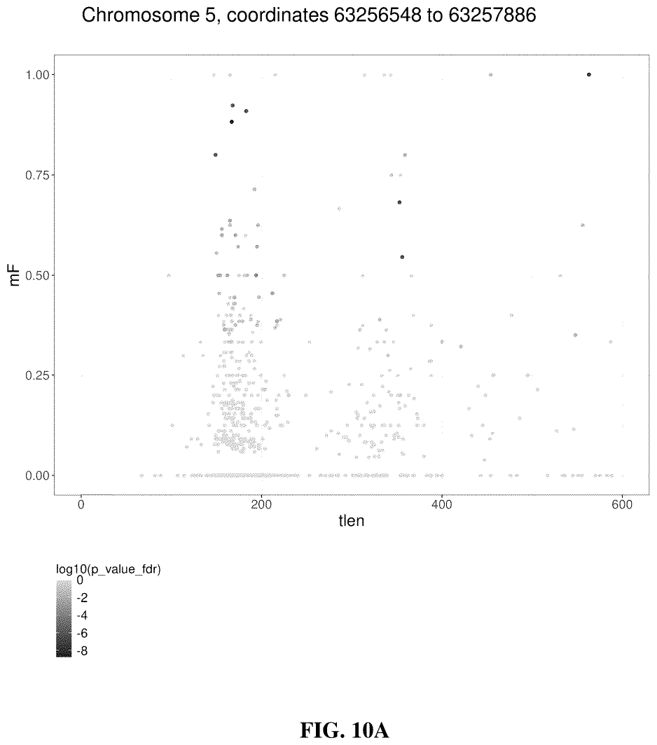

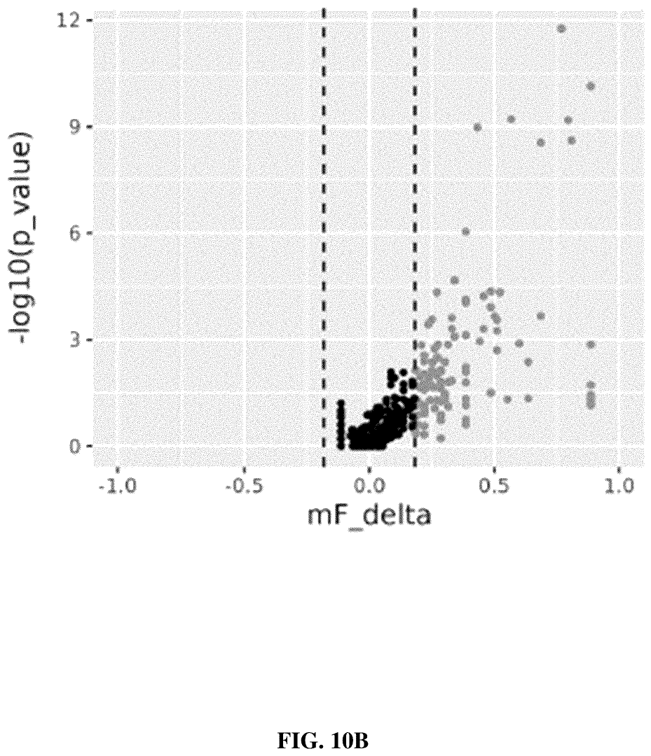

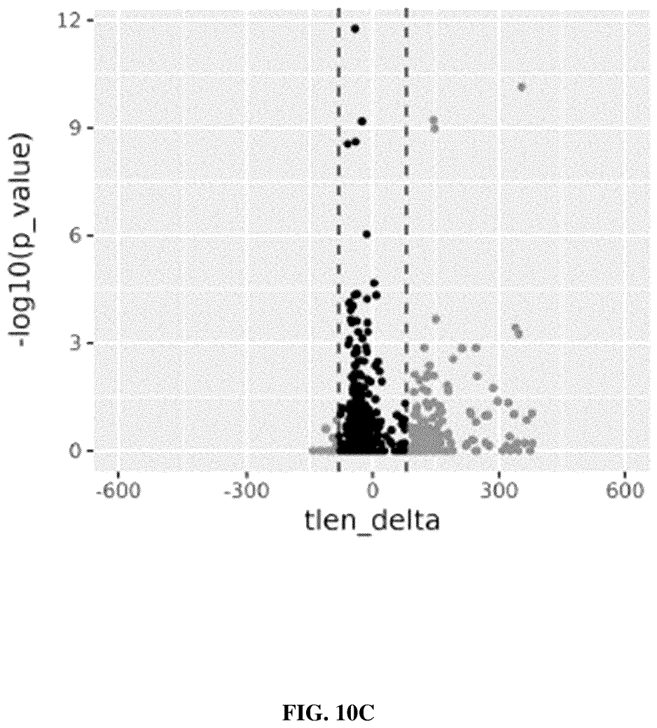

The three features for each sample cfDNA fragment, determined as described above, were compared within one subgenomic interval (corresponding to chromosome 5, coordinates 63256548 to 63/257,886) to examine the distribution of features for the sample cfDNA fragments. The methylation fraction values (“mF”) for the sample cfDNA fragments were compared against the fragment length values (“tlen”) for the sample cfDNA fragments (FIG. 10A). The p-values (“p value”) for the sample cfDNA fragments were compared against the methylation fraction differences (“mF_delta”) for the sample cfDNA fragments (FIG. 10B) and against the fragment length differences (“tlen_delta”) for the sample cfDNA fragments (FIG. 10C). These comparisons allowed for visualization of the pattern and distribution of features within one subgenomic interval for one sample, and indicated that the p-values, methylation fraction differences, and fragment length differences for cfDNA fragments could be used to distinguish between sample cfDNA fragments and control cfDNA fragments.

### Example 2—Filtering Sample cfDNA Fragments Using a Trained Filter Model

53 liquid biopsy samples from subjects with stage 3 or 4 colorectal cancer were sequenced to obtain sequencing data for sample cfDNA fragments. 114 control samples from control subjects without colorectal cancer were sequenced to obtain sequencing data for control cfDNA fragments. Comparisons were made, as described in Example 1, for each sample cfDNA fragment and corresponding control cfDNA fragments to calculate features for each sample cfDNA fragment: a p-value, a methylation fraction difference, and a fragment length difference.

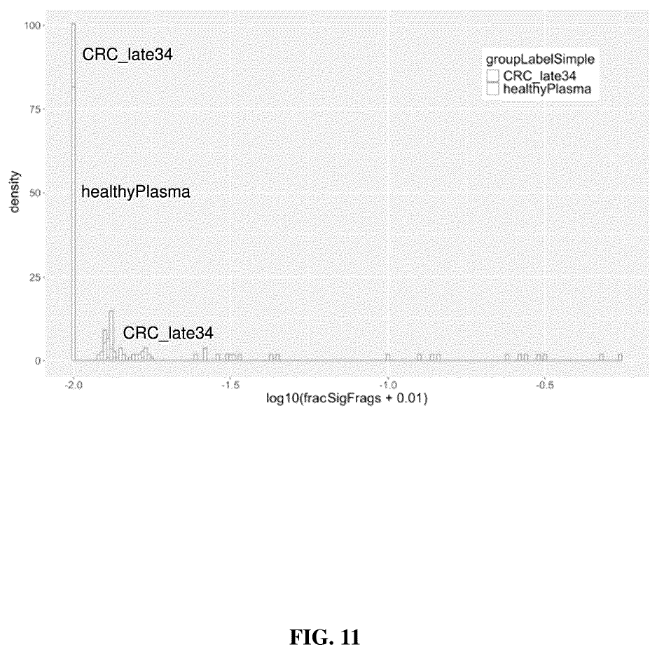

Within a subgenomic interval on chromosome 19 (coordinates 57182887 to 57183375), the following subgenomic interval-specific thresholds were determined using a grid search: a p-value threshold of less than 0.001, a methylation fraction difference threshold of greater than 0.75, and a fragment length difference threshold of greater than 0. The subgenomic interval-specific thresholds were then used to set one or more thresholds of a filter model to label cfDNA fragments as originating from tissue impacted by disease or originating from tissue not impacted by disease. The thresholds were able to distinguish between sample cfDNA fragments and control cfDNA fragments, determined by the fraction of sample cfDNA fragments that passed through the set thresholds compared against the fraction of control cfDNA fragments that passed through the set thresholds (FIG. 11).

### Example 3—Generating Test cfDNA Fragment Metrics

267 samples from subjects with colorectal cancer at different stages (stage 0, 1, 2, 3, 4) were sequenced to obtain sequencing data for test cfDNA fragments. 266 control samples from control subjects that were healthy or that did not have colorectal cancer were sequenced to obtain sequencing data for control cfDNA fragments. Comparisons were made, as described in Example 1, for each test cfDNA fragment and corresponding control cfDNA fragments to calculate features for each test cfDNA fragment: a p-value, a methylation fraction difference, and a fragment length difference. Features were calculated for test cfDNA fragments and corresponding control cfDNA fragments aligning with 106,000 subgenomic intervals.

The p-value, methylation fraction difference, and fragment length difference for each test cfDNA fragment were input into a trained filter model. The model was then used to filter the plurality of test cfDNA fragments. Test cfDNA fragments that passed through a predetermined number of filters in the model were labeled as originating from tissue impacted by disease. Test cfDNA fragments that did not pass through a predetermined number of filters in the model were labeled as not originating from tissue impacted by disease.

Six types of test cfDNA fragment metrics were computed for the filtered and labeled test cfDNA fragments (i.e., the fragments that passed through the thresholds of the trained filter model): (1) the fraction of test cfDNA fragments per subgenomic interval marked as significant (i.e., that passed through the filters of the trained filter model) relative to the total number of test cfDNA fragments; (2) the fraction of test cfDNA fragments marked as significant, aggregated over all subgenomic intervals; (3) the methylation fraction difference between test cfDNA fragments and corresponding control cfDNA fragments in subgenomic intervals where more test cfDNA fragments passed the thresholds than not; (4) a weighted methylation fraction for test cfDNA fragments that passed the thresholds; (5) a weighted methylation entropy for test cfDNA fragments that passed the thresholds; (6) the number of test cfDNA fragments remaining after filtering each subgenomic interval based on the thresholds of the trained filter model.

Using the methylation fraction difference metric computed for pre-filtered cfDNA fragments from the samples as described above (labeled as healthy, non-cancer, and colorectal cancer) in 106,000 subgenomic intervals, the performance of a machine learning classifier model using different feature selection strategies (Table 1, “nonCRCAnova”, “Anova ttest”, “Subclass Anova”) was evaluated. Results from the use of different feature selection strategies (based on pre-filtered cfDNA fragments) were then compared with benchmark results (from different subgenomic intervals) from a model previously considered as the best-performing model, using the weighted methylation fraction metric (Table 1, first column). The performance measures considered were AUC and sensitivity for various sample subclasses at 90% specificity. These performance measures were computed from the median AUC results among the set of results from a 3-fold CV with 6 iterations.

For the different feature selection strategies, one or more ANOVA tests were run, the top 1000 subgenomic intervals were selected, and the intersection of one or more sets of features was taken. The best AUC from the grid search is represented in the final column of Table 1 below, including only intervals within the 106,000 subgenomic intervals with a grid search AUC of greater than 0.7. These results represent improvements using different feature selection strategies for methylation fraction difference computed for pre-filtered cfDNA fragments, relative to a prior benchmark result as described above.

TABLE 1
Results from non-filtered cfDNA
fragments and filtered cfDNA fragments.

| | | | Intersect | | Filtered |
|---|---|---|---|---|---|
| | | Intersect CRC | Anova | | fragments |
| | Bench | nonCRCAnova | ttest CRC: | Subclass | (gridAUC > |
| | mark | Subclass | NonCRC | Anova | 0.7) |
| AUC | 0.86 | 0.93 | 0.93 | 0.92 | 0.88 |
| Stage 0 CRC | 0.54 | 0.60 | 0.60 | 0.54 | 0.37 |
| (advanced | | | | | |
| adenoma) | | | | | |
| (sensitivity at | | | | | |
| 90% | | | | | |
| specificity) | | | | | |
| Stage 1 CRC | 0.61 | 0.80 | 0.82 | 0.79 | 0.68 |
| (sensitivity at | | | | | |
| 90% | | | | | |
| specificity) | | | | | |
| Non-cancerous | 0.16 | 0.19 | 0.21 | 0.24 | 0.27 |
| non-advanced | | | | | |
| adenoma | | | | | |
| (sensitivity at | | | | | |
| 90% | | | | | |
| specificity) | | | | | |
| Total | 0.68 | 0.82 | 0.82 | 0.82 | 0.70 |
| sensitivity for | | | | | |
| all samples of | | | | | |
| all stages at | | | | | |
| 90% specificity | | | | | |

CRC = colorectal cancer.

Performance of the model in predicting the presence of disease and/or specific disease subtypes was compared between the weighted methylation fraction for cfDNA test fragments without filtering and the methylation fraction difference for test cfDNA fragments with filtering, using the same ANOVA-based feature selection strategy for each dataset (Table 2, indicating the best 1000 subgenomic intervals from the ANOVA tests). This analysis indicated better performance using filtered test cfDNA fragments relative to non-filtered test cfDNA fragments as applied for the detection of early stage colorectal cancer with advanced adenomas or not (Table 2).

TABLE 2
Model comparison of non-filtered test cfDNA
fragments and filtered test cfDNA fragments.

| | Weighted methylation | Filtered fragment |
|---|---|---|
| | fraction, ANOVA, non- | methylation fraction |
| | filtered fragments | difference, ANOVA |
| AUC | 0.86 | 0.93 |
| Earliest stage | 0.31 | 0.57 |
| colorectal cancer | | |
| (advanced adenoma) | | |
| fraction | | |
| Early stage colorectal | 0.53 | 0.84 |
| cancer 1 fraction | | |
| Non-cancerous non- | 0.35 | 0.27 |
| advanced adenoma | | |
| fraction | | |
| Sensitivity at 90% | 0.56 | 0.84 |
| specificity | | |

1. A method of training a filter model for filtering cfDNA fragments, comprising:
receiving, at one or more processors, sample sequencing data for a plurality of sample cfDNA fragments;
determining, using the one or more processors, for each of one or more sample cfDNA fragments from the plurality of sample cfDNA fragments, at least one of:
a value indicating that the sample cfDNA fragment originated from tissue not impacted by a disease;
a methylation fraction difference between a methylation fraction for corresponding control cfDNA fragments and a methylation fraction for the sample cfDNA fragment, and
a fragment length difference between an average length of corresponding control cfDNA fragments and a fragment length of the sample cfDNA fragment;
determining, using the one or more processors, for each of a plurality of subgenomic intervals, at least one of a subgenomic interval-specific value threshold, a subgenomic interval-specific methylation fraction difference threshold, and a subgenomic interval-specific fragment length difference threshold; and
setting, using the one or more processors, one or more thresholds of a filter model based on at least one of the determined subgenomic interval-specific value thresholds, the determined subgenomic interval-specific methylation fraction difference thresholds, and the determined subgenomic interval-specific fragment length thresholds.

2-5. (canceled)

6. The method of claim 1, wherein the sample sequencing data comprises, for each of one or more sample cfDNA fragments from the plurality of sample cfDNA fragments, at least one of a methylation status for one or more first selected methylation sites, a methylation fraction for one or more second selected methylation sites, and a fragment length.

7. The method of claim 6, wherein the value is a statistical value indicating the probability that the sample cfDNA fragment originated from tissue not impacted by a disease, and wherein the statistical value is based on the methylation status for the one or more first selected methylation sites in the sample cfDNA fragment and a methylation status for one or more third selected methylation sites in corresponding control cfDNA fragments.

8. The method of claim 6, wherein the methylation fraction difference is between the methylation fraction for one or more fourth selected methylation sites in the corresponding control cfDNA fragments and the methylation fraction for one or more second selected methylation sites in the sample cfDNA fragment.

9. A method of training a filter model for filtering cfDNA fragments, comprising:
receiving, at one or more processors, sample sequencing data for a plurality of sample cfDNA fragments obtained from a training sample of a training subject having a disease, the sample sequencing data comprising, for each of one or more sample cfDNA fragments from the plurality of sample cfDNA fragments, a methylation status for one or more first selected methylation sites, a methylation fraction for one or more second selected methylation sites, and a fragment length;
generating, using the one or more processors, for each of the one or more sample cfDNA fragments, a value indicating that the sample cfDNA fragment originated from tissue not impacted by the disease based on the methylation status for the one or more first selected methylation sites in the sample cfDNA fragment and a methylation status for one or more third selected methylation sites in corresponding control cfDNA fragments;
determining, using the one or more processors, for each of the one or more sample cfDNA fragments, a methylation fraction difference between a methylation fraction for one or more fourth selected methylation sites in corresponding control cfDNA fragments and the methylation fraction for the one or more second selected methylation sites in the sample cfDNA fragment;
determining, using the one or more processors, for each of the one or more sample cfDNA fragments, a fragment length difference between an average length of corresponding control cfDNA fragments and the fragment length of the sample cfDNA fragment;
determining, using the one or more processors, for each of a plurality of subgenomic intervals, a subgenomic interval-specific value threshold, a subgenomic interval-specific methylation fraction difference threshold, and a subgenomic interval-specific fragment length difference threshold based on the values, methylation fraction differences, and fragment length differences of each of the one or more sample cfDNA fragments that align with the subgenomic interval; and
setting, using the one or more processors, one or more thresholds of a filter model using the determined subgenomic interval-specific value thresholds, the determined subgenomic interval-specific methylation fraction difference thresholds, and the determined subgenomic interval-specific fragment length thresholds.

10. (canceled)

11. The method of claim 9, wherein:
a)_the one or more first selected methylation sites are all the methylation sites in the sample cfDNA fragment, and the one or more third selected methylation sites are all the methylation sites in the corresponding control cfDNA fragments;
(b) the one or more first selected methylation sites are all the methylation sites in a sample cfDNA fragment, and the one or more third selected methylation sites are selected based on a positional overlap with the one or more first selected methylation sites in the sample cfDNA fragment and alignment within a common subgenomic interval with the sample cfDNA fragment;
(c) the one or more third selected methylation sites are all the methylation sites in the corresponding control cfDNA fragments, and the one or more first selected methylation sites are selected based on a positional overlap with the one or more third selected methylation sites in the corresponding control cfDNA fragments and alignment within a common subgenomic interval with the corresponding control cfDNA fragments; or
(d) the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the sample cfDNA fragment and alignment within a common subgenomic interval with the sample cfDNA fragment.

12-14. (canceled)

15. The method of claim 9, wherein:
(a) the one or more second selected methylation sites are all the methylation sites in the sample cfDNA fragment, and the one or more fourth selected methylation sites are all the methylation sites in the corresponding control cfDNA fragments;
(b) the one or more second selected methylation sites are all the methylation sites in a sample cfDNA fragment, and the one or more fourth selected methylation sites are selected based on a positional overlap with the one or more second selected methylation sites in the sample cfDNA fragment and alignment within a common subgenomic interval with the sample cfDNA fragment;
(c) the one or more fourth selected methylation sites are all the methylation sites in the corresponding control cfDNA fragments, and the one or more second selected methylation sites are selected based on a positional overlap with the one or more fourth selected methylation sites in the corresponding control cfDNA fragments and alignment within a common subgenomic interval with the corresponding control cfDNA fragments; or
(d) the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the sample cfDNA fragment and alignment within a common subgenomic interval with the sample cfDNA fragment.

16-18. (canceled)

19. A method of labeling test cfDNA fragments from a test subject, comprising:
receiving, at one or more processors, test sequencing data for a plurality of test cfDNA fragments;
determining, using the one or more processors, for each of one or more test cfDNA fragments from the plurality of test cfDNA fragments, at least one of:
a value indicating that the test cfDNA fragment originated from tissue not impacted by the disease;
a methylation fraction difference between a methylation fraction for corresponding control cfDNA fragments and a methylation fraction for the test cfDNA fragment;
a fragment length difference between an average length of corresponding control cfDNA fragments and a fragment length of the test cfDNA fragment;
inputting, using the one or more processors, for each of the one or more test cfDNA fragments, at least one of the value, the methylation fraction difference, and the fragment length difference into a trained filter model;
filtering, using the one or more processors, the plurality of test cfDNA fragments using the filter model; and
labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

20. (canceled)

21. The method of claim 19, wherein the selected corresponding control cfDNA fragments are selected based on alignment within a common subgenomic interval with the test cfDNA fragment.

22-24. (canceled)

25. The method of claim 19, wherein the test sequencing data comprises, for each of one or more test cfDNA fragments from the plurality of test cfDNA fragments, at least one of a methylation status for one or more first selected methylation sites, a methylation fraction for one or more second selected methylation sites, and a fragment length.

26. The method of claim 25, wherein;
(a) the value is a statistical value indicating the probability that the sample cfDNA fragment originated from tissue not impacted by a disease, and wherein the statistical value is based on the methylation status for the one or more first selected methylation sites in the test cfDNA fragment and a methylation status for one or more third selected methylation sites in corresponding control cfDNA fragments; and/or
(b) the methylation fraction difference is between the methylation fraction for one or more fourth selected methylation sites in the corresponding control cfDNA fragments and the methylation fraction for one or more second selected methylation sites in the test cfDNA fragment.

27. (canceled)

28. A method of labeling test cfDNA fragments from a test subject, comprising:
receiving, at one or more processors, test sequencing data comprising, for each of one or more test cfDNA fragments from a plurality of test cfDNA fragments from a test sample from the test subject, a methylation status for one or more first selected methylation sites, a methylation fraction for one or more second selected methylation sites, and a fragment length;
generating, using the one or more processors, for each of the one or more test cfDNA fragments, a value indicating that the test cfDNA fragment originated from tissue not impacted by the disease based on the methylation status for the one or more first selected methylation sites in the test cfDNA fragment and a methylation status for one or more third selected methylation sites in corresponding control cfDNA fragments;
determining, using the one or more processors, for each of the one or more test cfDNA fragments, a methylation fraction difference between a methylation fraction for one or more fourth selected methylation sites in the corresponding control cfDNA fragments and the methylation fraction for the one or more second selected methylation sites in the test cfDNA fragment;
determining, using the one or more processors, for each of the one or more test cfDNA fragments, a fragment length difference between an average length of corresponding control cfDNA fragments and the fragment length of the test cfDNA fragment;
inputting, using the one or more processors, for each of the one or more test cfDNA fragments, the value, the methylation fraction difference, and the fragment length difference into a trained filter model;
filtering, using the one or more processors, the plurality of test cfDNA fragments using the filter model; and
labeling, using the one or more processors, test cfDNA fragments that pass through a predetermined number of filters in the filter model as originating from tissue impacted by the disease and test cfDNA fragments that do not pass through the predetermined number of filters in the filter model as not originating from the tissue impacted by the disease.

29. (canceled)

30. The method of claim 28, wherein:
(a) the one or more first selected methylation sites are all the methylation sites in the test cfDNA fragment, and the one or more third selected methylation sites are all the methylation sites in the corresponding control cfDNA fragments;
(b) the one or more first selected methylation sites are all the methylation sites in a test cfDNA fragment, and the one or more third selected methylation sites are selected based on a positional overlap with the one or more first selected methylation sites in the test cfDNA fragment and alignment within a common subgenomic interval with the test cfDNA fragment;
(c) the one or more third selected methylation sites are all the methylation sites in the corresponding control cfDNA fragments, and the one or more first selected methylation sites are selected based on a positional overlap with the one or more third selected methylation sites in the corresponding control cfDNA fragments and alignment within a common subgenomic interval with the corresponding control cfDNA fragments; or
(d) the one or more first selected methylation sites are selected based on a positional overlap with the one or more third selected methylation sites in the corresponding control cfDNA fragments and alignment within a common subgenomic interval with the corresponding control cfDNA fragments, and the one or more third selected methylation sites are selected based on a positional overlap with the one or more first methylation sites in the test cfDNA fragment and alignment within a common subgenomic interval with the test cfDNA fragment.

31-33. (canceled)

34. The method of claim 28, wherein:
(a) the one or more second selected methylation sites are all the methylation sites in the test cfDNA fragment, and the one or more fourth selected methylation sites are all the methylation sites in the corresponding control cfDNA fragments;
(b) the one or more second selected methylation sites are all the methylation sites in a test cfDNA fragment, and the one or more fourth selected methylation sites are selected based on a positional overlap with the one or more second selected methylation sites in the test cfDNA fragment and alignment within a common subgenomic interval with the test cfDNA fragment;
(c) the one or more fourth selected methylation sites are all the methylation sites in the corresponding control cfDNA fragments, and the one or more second selected methylation sites are selected based on a positional overlap with the one or more fourth selected methylation sites in the corresponding control cfDNA fragments and alignment within a common subgenomic interval with the corresponding control cfDNA fragments; or
(d) the one or more second selected methylation sites are selected based on a positional overlap with the one or more fourth selected methylation sites in the corresponding control cfDNA fragments and alignment within a common subgenomic interval with the corresponding control cfDNA fragments, and the one or more fourth selected methylation sites are selected based on a positional overlap with the one or more second methylation sites in the test cfDNA fragment and alignment within a common subgenomic interval with the test cfDNA fragment.

35-41. (canceled)

42. The method of claim 26, wherein generating the statistical value comprises:
aligning, using the one or more processors, the test cfDNA fragment and the plurality of corresponding control cfDNA fragments to a reference sequence to generate an alignment;
identifying from the alignment, using the one or more processors, corresponding control cfDNA fragments that positionally overlap with the test cfDNA fragment; and
comparing, using the one or more processors, the methylation status for the one or more first selected methylation sites in the test cfDNA fragment to the methylation status for the one or more third selected methylation sites in the corresponding control cfDNA fragments.

43. The method of claim 42, wherein comparing the methylation status for the one or more first selected methylation sites in the test cfDNA fragment to the methylation status for one or more third selected methylation sites in the corresponding control cfDNA fragments comprises determining a number of methylated sites in the one or more first selected methylation sites in the test cfDNA fragment, a number of unmethylated sites in the one or more first selected methylation sites in the test cfDNA fragment, a number of methylated sites in the one or more third selected methylation sites in the corresponding control cfDNA fragments, and a number of unmethylated sites in the one or more third selected methylation sites in the corresponding control cfDNA fragments.

44-45. (canceled)

46. The method of claim 19, further comprising generating one or more test cfDNA fragment metrics for the test cfDNA fragments labeled as originating from the tissue impacted by the disease, wherein the one or more test cfDNA fragment metrics comprises a fraction of test cfDNA fragments labeled as originating from the tissue impacted by the disease, a fraction of test cfDNA fragments labeled as originating from the tissue impacted by the disease within a common subgenomic interval, an average methylation fraction for test cfDNA fragments labeled as originating from the tissue impacted by the disease, an average methylation fraction difference for test cfDNA fragments labeled as originating from the tissue impacted by the disease, a number of test cfDNA fragments labeled as originating from the tissue impacted by the disease within a common subgenomic interval, a weighted methylation entropy of test cfDNA fragments labeled as originating from the tissue impacted by the disease, an average fragment length difference for test cfDNA fragments labeled as originating from the tissue impacted by the disease, an average fractional difference in fragment length of test cfDNA fragments labeled as originating from the tissue impacted by the disease, a number of test cfDNA fragments labeled as originating from the tissue impacted by the disease multiplied by an average methylation fraction difference of test cfDNA fragments labeled as originating from the tissue impacted by the disease, or a fraction of test cfDNA fragments labeled as originating from the tissue impacted by the disease multiplied by an average methylation fraction difference of test cfDNA fragments labeled as originating from the tissue impacted by the disease.

47. The method of claim 46, comprising inputting the one or more test cfDNA fragment metrics into a trained machine learning model configured to receive the one or more test cfDNA fragment metrics and predict whether the test subject has the disease.

48-69. (canceled)

70. The method of claim 1, wherein generating the value comprises:
aligning, using the one or more processors, the sample cfDNA fragment and the plurality of corresponding control cfDNA fragments to a reference sequence to generate an alignment,
identifying from the alignment, using the one or more processors, corresponding control cfDNA fragments that positionally overlap with the sample cfDNA fragment, and
comparing, using the one or more processors, the methylation status for the one or more first selected methylation sites in the sample cfDNA fragment to the methylation status for the one or more third selected methylation sites in the corresponding control cfDNA fragment.

71. The method of claim 70, wherein comparing the methylation status for the one or more first selected methylation sites in the sample cfDNA fragment to the methylation status for one or more third selected methylation sites in the corresponding control cfDNA fragments comprises determining a number of methylated sites in the one or more first selected methylation sites in the sample cfDNA fragment, a number of unmethylated sites in the one or more first selected methylation sites in the sample cfDNA fragment, a number of methylated sites in the one or more third selected methylation sites in the corresponding control cfDNA fragments, and a number of unmethylated sites in the one or more third selected methylation sites in the corresponding control cfDNA fragments.

72. (canceled)

73. The method of claim 1, wherein determining the subgenomic interval-specific value threshold, the subgenomic interval-specific methylation fraction difference threshold, and the subgenomic interval-specific fragment length difference threshold comprises:
determining a plurality of receiver operating characteristic (ROC) curves using a plurality of different subgenomic interval-specific value thresholds, a plurality of different subgenomic interval-specific methylation fraction difference thresholds, and a plurality of different subgenomic interval-specific fragment length difference thresholds; and
selecting the subgenomic interval-specific value threshold, the subgenomic interval-specific methylation fraction difference threshold, and the subgenomic interval-specific fragment length difference threshold providing the highest area under the curve (AUC) among the plurality of ROC curves.

74-124. (canceled)
